(19) United States
(12) Patent Application Publication
(10) Pub. No.: US 2017/0210820 A1
(43) Pub. Date: Jul. 27, 2017

(54) HUMAN ANTIBODIES AND PROTEINS

(71) Applicants: Timothy David Jones, Cambridge (GB); Matthew Paul Baker, Cambridge (GB)

(72) Inventors: Timothy David Jones, Cambridge (GB); Matthew Paul Baker, Cambridge (GB)

(21) Appl. No.: 15/471,290

(22) Filed: Mar. 28, 2017

Related U.S. Application Data
(63) Continuation of application No. 14/324,478, filed on Jul. 7, 2014, now abandoned.
Application No. 14/324,478 is a continuation of application No. 11/815,507, filed on Aug. 31, 2007, now abandoned.

(30) Foreign Application Priority Data
Feb. 3, 2005 (GB) 0502201.7
Feb. 16, 2005 (GB) 0503190.1
Apr. 5, 2005 (GB) 0506945.5

(51) Int. Cl. C07K 16/32; C07K 16/42; G01N 33/53; C07K 14/815; C07K 16/00; C07K 16/28; C07K 16/24

(52) U.S. Cl. CPC C07K 16/32; C07K 16/2896; C07K 16/4291; C07K 16/241; C07K 14/815; C07K 16/00; G01N 33/53; C07K 2317/622; C07K 2317/62; C07K 2317/56; C07K 2317/55; C07K 16/2863; C07K 2317/21; C07K 2317/24

(57) ABSTRACT

The present invention provides composite proteins, including antibodies, which show reduced immunogenicity. In particular, composite antibodies for use in humans are provided, in particular antibodies which have been modified to remove one or more T-cell epitopes. Methods for generating such proteins are also provided.

This application is a continuation of U.S. Ser. No. 14/324,478, filed Jul. 7, 2014, which is a continuation of U.S. Ser. No. 11/815,507, filed Aug. 31, 2007, now abandoned, entitled HUMAN ANTIBODIES AND PROTEINS, which is the National Stage Entry of PCT/GB2006/000355, filed Feb. 3, 2006, the contents of each of which are hereby incorporated herein by reference.

The present invention relates to generation of antibodies and proteins which combine two or more segments of amino acid sequence from a human antibody or protein within the final antibody or protein molecule. In particular, the present invention provides such combinations of sequence segments such that the number of T cell epitopes in the final antibody or protein molecule is reduced or avoided. The invention particularly relates to the generation of antibodies and proteins for use as pharmaceutical agents in humans or as in vivo diagnostic agents.

The last 20 years has seen great advances in the generation of recombinant monoclonal antibodies for use as potential pharmaceuticals in man. The techniques of chimerization, humanization and human antibody cloning either by phage display or transgenic mice have provided antibodies which are generally well tolerated when administered to man with less immunogenicity than with non-human monoclonal antibodies. However, several antibodies generated by these techniques have been shown to elicit immunogenicity in patients even where the genetic origins of such antibodies are human. For example, the human antibody Humira®, elicits immunogenicity in 12% of rheumatoid arthritis patients and the humanized antibody CAMPATH® elicits immunogenicity in about 50% of patients. Such induction of immunogenicity is likely to result from the presence, within the antibody variable region, of tracts of non-self amino acid sequences which, in some cases, can create T cell epitopes which induce T cell responses resulting in immunogenicity. There is therefore a need for improved techniques and antibody compositions which have a high human origin but which avoid, as much as possible, creation of sequences which might induce T cell responses.

The present invention provides methods and resultant antibody compositions whereby, for therapeutic use, such antibodies (herein termed “composite antibodies”) combine two or more segments of amino acid sequence from a human antibody within the final antibody molecule.

Thus, in a first aspect, the present invention provides modified antibody or antigen-binding fragment thereof wherein the heavy and light chain variable regions of the modified antibody or antigen-binding fragment are each composed of two or more segments of amino acid sequence from one or more other antibodies or antigen-binding fragments, whereby the segments are neither whole CDRs nor framework regions.

In the context of the present invention, the term “segments” refers to contiguous amino acid sequence found within an antibody molecule, such segments ranging in size from 2 to 125 amino acids long, preferably ranging from 2 to 31 amino acids long where such segments are neither whole CDRs nor whole framework regions. For therapeutic use, composite antibodies of the present invention will typically combine two or more segments of amino acid sequence from different human antibodies within the variable regions of the composite antibody. In particular, the present invention relates to composite antibody heavy and light chain variable regions (VH and VL respectively) where each VH and VL is composed entirely of segments of sequence from two or more human antibody variable regions and where typically each composite VH and VL includes segments of human variable region sequence positions corresponding to their positions in the source human antibody VHs and VLs, for example amino acids 1 to 10 in the composite VH sequence will derive from amino acids 1 to 10 in a human antibody. Alternatively, segments of human VH or VL sequence in the composite antibody may be positioned at any sequence location irrespective of the sequence position in the source human antibody VH or VL. The source human antibody VHs and VLs will be any existing human antibody variable (V) region amino acid sequence, for example as provided in databases of human monoclonal antibody V region sequences, and may include sequences from affinity-matured antibodies with V region somatic mutations and other variations differing from germ-line, sequences from germ-line V regions, sequences from artificially constructed antibody V regions created from segments of sequence from antibodies of the species such as antibodies with a set of fixed V region frameworks but with variable CDRs, sequences selected from human antibody libraries such as phage display libraries, and sequences of human antibodies derived from transgenic animals expressing genes encoding human antibodies or antibody fragments.

In a preferred embodiment of the present invention, composite antibodies of the invention for therapeutic use are constructed by combining multiple human VH and VL sequence segments in combinations which limit or avoid human T cell epitopes in the final composite antibody V regions.

Human T cell epitopes in this respect are amino acid sequences which can bind to human MHC class II molecules and, through presentation to CD4⁺ T cells, induce a helper T cell response. Human VH and VL sequence segments and combinations of segments can be chosen which limit or avoid T cell epitopes in the final composite antibody. This can be achieved by use of segments which do not contain T cell epitopes, such as from human germ-line sequences, and by joining of adjacent segments to create a new sequence which does not contain T cell epitopes, for example by creation of a non-MHC binding sequence at the junction of two segments, by creation of another human germ-line sequence, or by creation of a sequence which does not induce a helper T cell response despite a non-germ-line sequence.

In another preferred embodiment of the present invention, additional amino acid sequences can be added or created within the composite antibody molecules which provide for one or more regulatory T cell epitopes (“Tr epitopes”). For the purpose of the invention, Tr epitopes are MHC binding peptides which stimulate CD4⁺ CD25⁺ T cells with the ability to regulate immune responses by the secretion of inhibitory cytokines such as IL-10 and TGF-β, as well as contact dependent mechanisms. As such, within the scope of the invention, regulatory T cell epitopes can include peptides shown to induce one or more activities in vitro or in vivo which could contribute to regulation of immune responses under certain conditions. For example, regulatory T cell epitopes will include peptides with the action of inducing or activating CD4⁺ CD25⁺ T cells, with the action of inducing release of inhibitory cytokines such as IL-10 and/or TGF-β, or with other measurable immunosuppression-related activities either in vitro or in vivo, in all cases where the actions are related to the action of CD4⁺ CD25⁺ T cells. Thus, such Tr epitopes can provide an additional measure to limit or avoid immunogenicity in the composite antibody. Tr epitopes can be introduced into the composite antibody VH or VL by incorporation of segments of human VH and VL containing these epitopes or by creation of such epitopes via combination of two or more human sequence segments or by screening for new Tr epitopes, for example from peptides corresponding to segments of human antibody or protein sequence, for induction or activation of CD4⁺ CD25⁺ T cells, for example by measurement of release of inhibitory cytokines such as IL-10 and/or TGF-β (e.g. Hall et al., Blood, vol.100 (2002) p4529-36). Alternatively, known Tr epitopes can be incorporated within composite antibody V regions at positions within VH and/or VL which do not inhibit binding or function or expression of the composite antibody or can be incorporated at one terminus of the composite VH or VL sequence, for example at the N terminus of VH. Alternatively, Tr epitopes can be incorporated into one or both constant regions of a composite antibody at locations which do not interfere with function of the composite antibody (e.g. within the hinge regions) or cause some other deleterious effect such as lack of expression. Alternatively, for one or both of composite VH and VL's within antibody fusion proteins, antibody conjugates, Fab and Fv-type forms (including single chain antibodies (SCAs) with VH and VL linked), single domain antibodies, or homodimeric antibodies, Tr epitopes can be incorporated at locations which do not interfere with function of the composite antibody or cause some other deleterious effect such as lack of expression. For example, in SCAs, an especially preferred location for a Tr epitope is within the linker region joining VH and VL. Optimally, Tr epitopes will be flanked by appropriate sequences to optimise the release and presentation of regulatory T cell epitopes on MHC class II molecules, for example by flanking the epitope with sequences that are sensitive to the action of endocytic proteases. Typically, flanking residues at positions ranging from P-20 to P30 (with the core nonomer defined as P1-P9) that will target the action of proteases during antigen processing are introduced, if necessary using additional segments of human antibody sequence.

As discussed herein, the present invention also provides methods for the production of modified or composite antibodies. Thus, in another aspect, the present invention provides a method for producing a modified antibody comprising the steps;
(1) preparing antibody variable region genes by combining segments of amino acid sequence from a range of other antibody variable regions in order to generate a library of different variable region genes
(2) cloning the library of antibody variable region genes into an expression vector
(3) screening the library of antibody variable regions and recovering members of the library with desirable properties

In a first preferred method ‘A’ of the present invention, a library of composite human antibodies is generated and screened for antibodies with desirable properties such as binding to a specific antigen. This method involves 6 steps as follows;
(1) design of composite VH and VL genes
(2) cloning of composite VH and VL genes
(3) expression of composite VH and VL genes
(4) screening and selection of composite antibodies with desirable properties
(5) optimisation of lead composite antibodies
(6) (optional) avoidance of T cell epitopes

For step (1), the library of composite VH and VL sequences are designed by selecting segments of VH and VL sequence from known human V region sequences such as those available in the Kabat antibody database (www.bioinf.org.uk/abs/simkab.html), the NCBI database (www.ncbi.nlm.nih.gov) and from protein databases, such as UniProt (www.ebi.uniprot.org) and PRF/SEQDB (www.prf.or.jp). In addition, these can be supplemented by collection of human VH and VL sequences by direct sequencing of amplified VH and VL mRNA from one or more individual donors. Various combinations of sequence segments can be considered for design of VH and VL genes. One method used is to fix the length of the composite VH and VL sequences and to design these using fixed length sequence segments from corresponding Kabat numbering positions in different human V regions.

For example, the library would comprise VH and VL regions of 121 and 107 amino acids respectively and would include, for example, an assortment of different segments for VH amino acids 1-27 using Kabat numbering. For VH with CDRs corresponding to Kabat numbering CDR1:30-35, CDR2:50-66 and CDR3:95-106, sequence segments for the following Kabat positions are used as one option: 1-27, 28-31, 32-36, 37-42, 43-50, 51-56, 57-60, 61-63, 64-69, 70-82a, 82b-96, 97-98, 99-101, 102-117. For VL with CDRs corresponding to Kabat numbering CDR1:24-34, CDR2: 50-56, CDR3:89-97, sequence segments for the following Kabat positions are used as one option: 1-22, 23-27, 28-30, 31-33, 34-35, 36-47, 48-52, 53-55, 56-59, 60-87, 88-92, 93-94, 95-107. Therefore, in this example, composite VHs are composed of 14 human segments and composite VLs are composed of 13 human segments. In practice, a computer program is used to generate combinations of these segments. Preferably, the program includes an algorithm to avoid non-preferred combinations of certain segments which might, for example, avoid certain canonical structures of CDRs or which might disrupt VH and/or VL folding or VH/VL interaction. As an optional addition, the program could include an algorithm to limit the number of T cell epitopes formed by the combination of sequence segments (see in silico methods in step (6) below).

For step (2), having designed a library of composite human sequences, composite VH and VL genes are then generated preferably using synthetic oligonucleotides. Typically, synthetic oligonucleotides encoding longer segments of V region sequence will be ligated to a mixture of oligonucleotides which encode two or more consecutive segments of V region sequence. Alternatively, composite V regions could be assembled by other methods such as overlapping PCR or other amplification techniques using existing human VH and VL genes as templates. For example, using PCR, small segments of V regions can be amplified separately and then joined by overlapping PCR reactions.

In other methods, mixed synthetic oligonucleotides can be produced to create a range of sequence segments preferably using doping methods to enrich for sequences encoding specific V region segments. Composite human VH and VL genes with extensive variability of human V region segment representation can be assembled in many ways using techniques known to those skilled in the art such as those described in Molecular Cloning: A Laboaratory Manual; 3^{rd} Ed., vols. 1-3 (2001) Cold Spring Harbor Laboratory Press and using standard PCR methods for immunoglobulins such as those described in Orlandi et al., Proc Natl Acad Sci USA.,86 (1989) 3833-3837.

For step (3), once composite human VH and VL genes are generated, these can be cloned into a variety of expression vectors for production of either complete antibody molecules or antigen-binding fragments such as Fv's, Fab's, Fab2, SCAs, single domain antibodies (e.g. comprising VHs only) and multimeric derivatives of each of these. Alternatively, VH and VL genes can be fused to genes encoding other molecules to generate fusion proteins. Also included might be sequences encoding detectable markers such as poly-histidine tags at the C terminus of one chain of an Fv or Fab. Expression vectors include those for expression in mammalian cells, bacterial cells, bacteriophage, yeast, fungus and other micro-organisms. Such vectors also include those for expression in vivo from transgenic animals and those for expression using in vitro systems such as in vitro translation using ribosome preparations.

For step (4), screening of libraries of composite human antibodies is usually for binding to one or more specific antigens of interest. There are many screening methods known to those skilled in the art, the selection of which will depend on the form of expression of the composite human antibodies and the composition of the antibody molecules i.e. complete antibody or Fab, Fv, SCA, single domain antibody etc. In some cases where an existing antibody is available which binds to the antigen of interest, either VH or VL from this antibody may be combined with the composite human VL or VH respectively and tested for binding.

Screening methods will range from immobilising individual members of the library or pools of such members on a solid phase to immobilising the antigen of interest either individually or in pools. Where antibodies are immobilised, the antigen of interest is then added and is either detectable directly or indirectly by addition of one or more additional reagents. For example, if the antigen is a fusion protein or conjugate with an enzyme such as alkaline phosphatase, detection can be achieved by subsequent addition from a wide range of substrates which produce colour, fluorescence or chemiluminescent signals. Where antibody pools are immobilised in one location (e.g. the well of a microtitre dish) and a signal results from addition of antigen, this pool can then be dereplicated prior to rescreening of either individual members of the pool or smaller pools. Where the antigen of interest is immobilised, the composite antibody library may be screened in several ways ranging from addition of individual antibodies to the antigen of interest which is immobilised at a specific location, to addition of pools of antibody, to addition of the whole composite library and subsequent recovery of antibodies bound to the antigen of interest. In the last case, a common strategy is to immobilise antigen on a solid phase such as in a column or on beads, to add the library, to subsequently wash the solid phase for example with a low salt buffer (to detach loosely associated members of the library), and to then elute antibodies which bind to the antigen using, for example, a high salt buffer. Common formats for expression of members of the library for this purpose are phage display, yeast display, ribosome display and bead display, in each case where nucleic acid encoding composite VH and VL chains remains attached to the composite V region which binds to the antigen.

Screening methods will also include functional or biological tests which may be substituted for direct antigen binding tests where a functional or biological activity is measured such as in vitro tests involving cell growth, cell growth inhibition, cell differentiation or cell migration, or alternative in vivo tests involving measuring responses to the antigen at the level of the whole organism, for example changes in blood cell counts in a mouse or growth inhibition of a transplanted tumour.

For step (5), following selection of one or more “lead” composite human antibodies with desirable properties such as binding to an antigen of interest, optionally the properties of the lead antibody may be improved, for example by increasing affinity for binding to the antigen or fusing the antibody to an additional moiety. Increased affinity may be achieved by mutagenesis of composite variable region sequences in order to select for mutations in the selected composite V region sequences which increase or alter binding in a desirable way. The present invention includes novel methods for mutagenesis of variable region sequence by replacing one or more individual V region sequence segments from the lead antibody with corresponding sequence segments from one or more human antibody sequences. In particular, segments overlapping with or within CDR region may be replaced by one or more alternative segments from other human antibodies including segments of different lengths. Within the scope of the invention, specific segments may be included from human antibodies with related properties to the selected lead antibody, for example from antibodies which bind to the same antigen, or from non-human antibodies with related properties, or from human antibodies with sequence segments which retain certain key amino acids which appear important for function in a non-human antibody with related sequence. One or more composite human antibodies subject to such mutagenesis can then be screened for improved properties.

For the optional step (6), following selection of a lead composite human antibody, T cell epitopes are limited or avoided by, where required, exchanging V region segments contributing to or encoding a T cell epitope with alternative segments which avoid T cell epitopes. Such T cell epitopes can be detected by a range of methods. For example, peptides corresponding to one or more loci in the composite V region sequence can be synthesised and tested in T cell assays to determine the presence of T cell epitopes. Typically such peptides will be 15 amino acids in length and, where it is desirable to test a longer contiguous V region sequence, overlapping peptides from the sequence such as 15 mers with 12 amino acid overlaps are used. For detection of T cell epitopes, a range of different T cell assays can be used for measurement of activation or proliferation of CD4⁺ T cells such as those measuring cytokine release, proliferation (for example, by uptake of 3H-thymidine), Ca2⁺ flux, surface marker expression, gene transcription etc.

Alternatively, overlapping peptides corresponding to the composite V region sequences are analysed for binding to human MHC class II molecules either using in vitro methods or in silico methods, in each case to determine potential T cell epitopes i.e. MHC binding peptides which may induce a T cell response. In silico methods will include methods involving modelling of peptide-MHC class II binding interactions, methods involving identification of motifs common for binding to MHC class II and methods using databases of peptides or specific amino acids within peptides with known in vitro MHC binding properties. Other methods can be used such as producing longer peptides from composite V region sequences or whole antibodies containing composite V region sequences and testing these in T cell assays or in MHC binding assays, for example by testing for MHC-peptide tetramers, or by searching the proposed or constructed sequences in a database of known human T cell epitopes. Avoidance of T cell epitopes in composite human V regions can also be assisted by avoidance of MHC class II binding motifs or avoidance of particular amino acids which anchor the binding of peptides to MHC class II. In the preferred method for avoidance of T cell epitopes from one or more lead composite human antibodies, in silico methods are initially applied to analyse the composite human antibody V regions for potential T cell epitopes and, where these are identified, new segments of human VH or VL sequence are introduced to avoid these epitopes and to avoid introduction of new T cell epitopes.

Following any such introduction of new human V region segments and rescreening of such modified lead composite human antibodies for desirable properties, one or more final lead composite human V region can then be further tested in human T cell assays either by testing overlapping peptides typically of 15 to 45 amino acids in length, for example 15 mer peptides with 12 amino acid overlaps from the composite human V region sequences (whole V regions or parts thereof) or by testing whole composite human antibodies directly in human T cell assays. A final analysis using T cell assays for testing whole composite human antibody is preferred allowing for direct testing for T cell activation against the whole antibody.

In a second preferred method ‘B’ of the present invention, a library of composite human antibodies is generated to include desirable amino acids from one or more reference antibodies with desirable properties. This method involves 7 steps as follows;
(1) sequence analysis of one or more reference antibodies
(2) design of composite VH and VL genes
(3) (optional) avoidance of T cell epitopes
(4) cloning of composite VH and VL genes
(5) expression of composite VH and VL genes
(6) screening and selection of composite antibodies with desirable properties
(7) optimisation of lead composite antibodies including optional avoidance of T cell epitopes

In step (1), typical reference antibodies will be rodent, especially mouse, with properties and/or binding specificities which are desirable in a human form of antibody. Where one or more reference antibody V region sequences are available, these are analysed to determine sequences of the CDRs and to identify amino acids which might be important for the desirable properties of the antibody such as binding specificity. For a reference antibody, such analysis is performed, for example, by alignment of the reference V region sequences with other sequences of the same species and also, if the reference antibody is non-human, human V region sequences. Such aligments are performed, for example, using the program CLUSTAL (Thompson et al., Nucleic Acids Res. 22 (1994) p4673-80). Such alignments can identify unusual or rare amino acids in the V region of the reference antibody and homologous V region families. In addition; conserved V region structures such as canonical structures of the CDRs can be identified using, for example, the Protein Data Bank (Berman et al. The Protein Data Bank, Nucleic Acids Research, 28 (2000) 235-242). In addition, the reference antibody variable regions can be modelled, where a structure is not known, using modelling software such as MODELLER (Sali and Blundell, J. Mol. Biol. 234 (1993) p779-815) and, in some cases, models of antibody-antigen interactions can be generated. Such analyses of the reference antibody V regions are used to guide on selection of segments of human V region sequence for the composite human antibody.

For step (2), having determined amino acids which might be important for the desirable properties of the composite human antibody, segments of human V region sequences are then selected to include some or all of these amino acids. A library of composite human V region sequences is thereby designed including selected segments with typically one or more alternative human V region segments at particular loci where the effect of such segments on properties of the composite human antibody is uncertain. Such composite human antibody sequences can be further analysed as with the reference antibody(s) by alignment with other human antibody sequences and conserved structures and, in addition, further modelling of the structure of composite human antibody V regions can be undertaken in order to refine, as required, the combinations of human V region segments used in the composite human antibodies to avoid defects in protein structure, intermolecular and intramolecular interactions within composite V regions, and incorrect structural orientations of important amino acids.

For the optional step (3), as an additional criteria for selection of segments, those segments or combinations of segments which limit or avoid T cell epitopes in the final composite human V regions are selected. T cell epitopes are analysed by the methods described in method A, step (6) above using in silico or in vitro methods, preferably by use of in silico methods at the stage of designing composite human V region sequences.

For step (4), having designed a library of composite human sequences, composite VH and VL genes are then generated preferably using synthetic oligonucleotides. Typically, synthetic oligonucleotides encoding longer segments of V region sequence will ligated to a mixture of oligonucleotides which encode alternative segments of sequence to generate different members of the library of composite human V regions. Alternatively, each member of the library of composite human V regions will be generated separately using oligonucleotides encoding the sequence of the specific human V region. Alternatively, composite V regions can be assembled by other methods such as overlapping PCR or other amplification techniques using existing human VH and VL genes as templates or using one or more reference antibody V region genes as template.

Steps (5) and (6) for method B are as described in method A, steps (4) and (5).

Optional step (7) will be employed as in method A, step (6) where further avoidance of T cell epitopes is required in the lead composite human antibody(s). A final analysis using T cell assays for testing whole composite human antibody is preferred allowing for direct testing for T cell activation from the whole antibody.

It will be understood to those skilled in the art that, in addition to methods A and B, there will be other methods for creating and testing composite human antibodies and for optimising the properties of such antibodies. Composite human antibodies of the present invention are new and, as a result of the total human origin of the V regions, should be less immunogenic in humans than other antibodies containing non-human sequences. Additional optional features of composite human antibodies, namely the avoidance of T cell epitopes and/or the addition of Tr epitopes, may also contribute to lower immunogenicity. It will be understood by those skilled in the art that the object of lower immunogenicity may be achieved using less preferred composite antibodies containing V regions without all human sequence segments, for example composite human antibodies including segments at sequence positions in the composite antibody different from their sequence positions in the source human antibody, composite antibodies with only partial incorporation of segments of human V region sequence, composite antibodies with segments of non-human sequence, or composite antibodies with human sequence which has been mutated, for example to increase binding affinity to an antigen or to avoid a T cell eptiope.

It will be understood that V region sequence segments and their combinations within composite human antibodies might be selected to meet a range of criteria including the optional avoidance of T cell epitopes as above. For example, segments of human V region sequence and combinations thereof can be selected for avoidance of B cell epitopes and other epitopes such as MHC class I-restricted epitopes, for avoidance of amino acid sequences which might be deleterious to expression of composite antibodies, for avoidance of sequences which might direct inappropriate modification of composite antibodies such as N-glycosylation, for inclusion of certain functions such as inclusion of helper T cell epitopes and/or B cell epitopes (for example, in vaccine applications), for subsequent conjugation to other moieties such as one or more surface lysine residues, and for a range of other criteria.

It will also be understood by those skilled in the art that, in addition to human, composite antibodies with V region segments derived from other species either wholly or in part can be generated and should be considered within the scope of the invention. For example, for studies in mice, composite mouse antibodies can be generated comprising V region sequence segments wholly or partly of mouse origin.

The present invention also applies to proteins other than antibodies whereby, for therapeutic use, such proteins (herein termed “composite proteins”) combine two or more segments of amino acid sequence from a human protein within the final protein molecule.

Thus, in a further aspect, the present invention provides a modified protein having improved immunogenicity through insertion of one or more segments of amino acid sequence.

In relation to proteins, the term “segments” refers to contiguous amino acid sequence found within a protein molecule, such segments ranging in size from 2 to 250 amino acids long. For therapeutic use, composite proteins of the present invention will typically combine two or more segments of amino acid sequence from different human proteins within the composite protein. In particular, the present invention relates to composite proteins with insertions composed entirely of segments of sequence from two or more human proteins. Where human proteins exist with homology to the composite protein or with homologous regions to regions of the composite protein, segments of human protein sequence at sequence positions in the composite protein sequences corresponding to their sequence positions in the source human protein may be used, for example amino acids 1 to 10 in the composite protein sequence will derive from amino acids 1 to 10 in a source human protein. Alternatively, segments of human protein sequence may be positioned in the composite protein at any sequence location in the composite protein irrespective of the sequence position in the source human protein. The source human proteins will be any existing human protein amino acid sequence, for example as provided in databases of human protein sequences, and may include sequences from naturally mutated or rearranged forms of the human protein and other variations differing from germ-line, sequences from artificially constructed human-derived proteins and sequences derived from human genes or RNA whether the corresponding proteins are expressed or not.

In a preferred embodiment of this aspect of the present invention, composite proteins for therapeutic use are constructed by combining or inserting human protein sequence segments in combinations which limit or avoid human T cell epitopes in the final composite protein. A preferred aspect of the invention as applied to composite proteins is to modify an existing reference protein such as a non-human protein by insertion of human protein sequence segments in order to limit or avoid T cell epitopes in the final composite protein.

In a preferred method of the present invention for generation of composite proteins, a library of composite human proteins is generated to include desirable amino acids from one or more reference proteins with desirable properties such as an absence of T cell epitopes. This method involves 7 steps as follows;
(1) sequence analysis of one or more reference proteins including optional analysis of T cell epitopes
(2) design of composite protein genes
(3) (optional) avoidance of T cell epitopes
(4) cloning of composite protein genes
(5) expression of composite protein genes
(6) screening and selection of composite proteins with desirable properties
(7) optimisation of lead composite proteins including optional avoidance of T cell epitopes

In step (1), typical reference proteins will be non-human with properties which are desirable in a composite protein. For therapeutic application, typically the reduction or elimination of immunogenicity in the composite protein will be an objective. Where one or more reference protein sequences are available, these are analysed to identify amino acids which might be important for the desirable properties of the protein. In addition, any known structure of the reference protein can be analysed or, alternatively, a structure modelled using modelling software. Where homologues of the reference protein are available, either interspecies or intraspecies, these can be sometimes be used to determine relationships between sequence differences and differences in properties between homologues. Where the protein interacts with another molecule, models of this interaction can sometimes be generated and amino acids important for the interaction determined. As an optional addition to step 1, the sequence location of T cell epitopes in the reference protein are determined, in particular using in vitro human T cell assays as detailed for composite human antibodies above. Alternatively, in silico methods for analysing T cell epitopes can be used. Such analyses of the reference proteins are used to guide on segments of human protein sequence selected for the composite protein. For composite proteins where a reduction or elimination of immunogenicity compared to a reference protein, especially non-human, is the objective, commonly one or more human sequence segments corresponding to locations of T cell epitopes will be used in the composite protein in combination with segments of sequence from the reference protein from other locations without T cell epitopes.

For step (2), having determined amino acids which might be important for the desirable properties of the composite protein, segments of protein sequences are then selected to include some or all of these amino acids. A library of composite human protein sequences is thereby designed including selected segments with typically one or more alternative human protein segments at particular loci where the effect of such segments on properties of the composite protein is uncertain. Such composite protein sequences can be further analysed as with the reference protein by alignment with any homologues or by modelling of the structure of composite proteins or by other analyses in order to refine, as required, the combinations of human protein segments used in the composite human proteins to avoid defects in protein structure and incorrect structural orientations of important amino acids.

For the optional step (3), as an additional criteria or only criteria for selection of segments, those segments or combinations of segments which limit or avoid T cell epitopes in the final composite proteins are selected. T cell epitopes are analysed by the methods described for composite human antibodies above using in silico or in vitro methods.

For step (4), having designed a library of composite proteins, composite protein genes are then generated preferably using synthetic oligonucleotides. Typically, synthetic oligonucleotides encoding longer segments of protein sequence will ligated to a mixture of oligonucleotides which encode alternative segments of sequence to generate different members of the library of composite proteins. Alternatively, each member of the library of composite proteins will be generated separately using oligonucleotides encoding the sequence of the specific composite protein. Alternatively, composite proteins can be assembled by other methods such as overlapping PCR or other amplification techniques using existing human protein genes as templates or using one or more reference protein genes as template.

For step (5), screening of libraries of composite proteins is usually for one or more desirable properties of the composite protein. There are many screening methods known to those skilled in the art, the selection of which will depend on the form of expression of the composite proteins and the protein function. Screening methods will range from immobilising individual members of the library or pools of such members on a solid phase, to screening member of the library in solution phase, to immobilising another molecule with which the composite protein is designed to interact by binding either individually or in pools. Screening methods may also include functional or biological tests where a functional or biological activity is measured such as in vitro tests involving cell growth, cell growth inhibition, cell differentiation or cell migration, or alternative in vivo tests involving measuring responses to the composite protein at the level of the whole organism, for example changes in blood cell counts in a mouse or growth inhibition of a transplanted tumour.

For step (6), following selection of one or more “lead” composite proteins with desirable properties, optionally the properties of the lead protein may be improved, for example by increasing the specific activity of an enzyme or by increasing the binding of a protein ligand to a receptor. An improvement in properties may be achieved by mutagenesis of composite protein sequences in order to select for mutations which alter properties of the composite protein in a desirable way. The present invention includes novel methods for mutagenesis of a protein sequence by replacing one or more individual protein sequence segments from the protein with sequence segments from one or more human protein sequences. One or more composite proteins subject to such mutagenesis can then be screened for improved properties.

For the optional step (7), following selection of a lead composite protein, T cell epitopes are limited or avoided by, where required, exchanging protein sequence segments contributing to or encoding a T cell epitope with alternative segments which avoid T cell epitopes. Such T cell epitopes can be detected by a range of methods. For example, peptides corresponding to one or more loci in the composite protein can be synthesised and tested in T cell assays to determine the presence of T cell epitopes. Typically such peptides will be 15 amino acids in length and, where it is desirable to test a longer contiguous sequence, overlapping peptides from the sequence such as 15 mers with 12 amino acid overlaps are used. Alternatively, overlapping peptides corresponding to the composite protein sequences are analysed for binding to human MHC class II molecules either using in vitro methods or in silico methods, in each case to determine potential T cell epitopes i.e. MHC binding peptides which may induce a T cell response. In silico methods will include methods involving modelling of peptide-MHC class II binding interactions, methods involving identification of motifs common for binding to MHC class II and methods using databases of peptides or specific amino acids within peptides with known in vitro MHC binding properties. Other methods can be used such as producing longer peptides from composite protein sequences or whole composite proteins and testing these in T cell assays or in MHC binding assays on antigen presenting cells. Avoidance of T cell epitopes in composite proteins can also be assisted by avoidance of MHC class II binding motifs or avoidance of particular amino acids which anchor the binding of peptides to MHC class II. In the preferred method for avoidance of T cell epitopes from one or more lead composite proteins, in silico methods are initially applied to analyse the composite protein for potential T cell epitopes and, where these are determined, new segments of human protein sequence are introduced to avoid these epitopes and to avoid introduction of new T cell epitopes. Following such introduction of new human segments if required to avoid T cell epitopes and rescreening for modified lead composite proteins for desirable properties, one or more final lead composite proteins can optionally tested in human T cell assays either by testing overlapping peptides typically of 15 to 45 amino acids in length, for example 15 mer peptides with 12 amino acid overlaps from the composite protein sequences (whole proteins or parts thereof) or by testing whole composite proteins directly in human T cell assays. A final analysis using T cell assays for testing whole composite protein is preferred allowing for direct testing for T cell activation from the whole protein.

It will be understood to those skilled in the art that there will be other methods for creating and testing composite proteins and for optimising the properties of such proteins. Composite proteins of the present invention are new and, where used for therapeutic purposes, the human origin of some or all protein sequence segments should render the composite protein less immunogenic in humans than other comparable or non-human reference proteins containing non-human sequences. Additional optional features of composite proteins, namely the avoidance of T cell epitopes and/or the addition of Tr epitopes, may also contribute to lower immunogenicity. It will be understood by those skilled in the art that the object of lower immunogenicity may be achieved using composite proteins without all human sequence segments and may also include composite proteins with human sequence segments which have been mutated to eliminate a T cell eptiope or segments of non-human protein homologous to the reference protein. It will be understood that protein segments and their combinations within composite proteins might be selected to meet a range of criteria including the optional avoidance of T cell epitopes. For example, segments of human protein sequence and combinations thereof can be selected for avoidance of B cell epitopes and other epitopes such as MHC class I-restricted epitopes, for avoidance of amino acid sequences which might be deleterious to expression of composite proteins, for avoidance of sequences which might direct inappropriate modification of composite proteins such as N-glycosylation, for inclusion of certain functions such as inclusion of helper T cell epitopes and/or B cell epitopes (for example, in vaccine applications), for subsequent conjugation to other moieties, and for a range of other criteria.

It will also be understood by those skilled in the art that, in addition to human, composite proteins with sequence segments derived from other species either wholly or in part can be generated and should be considered within the scope of the invention. For example, for studies in mice, composite proteins including mouse protein sequence segments can be generated. It will also be understood that composite proteins can include protein sequence segments from one species combined with other protein sequence segments from homologous proteins within the same species. For example, the invention will include construction of plant type I RIPs (ribosome inhibitory proteins) where a RIP is assembled using sequence segments from the numerous plant type I RIP sequences available. Such composite RIPs would be assembled by introducing combinations of sequence segments which would retain RIP activity and, if for use in humans, would include avoidance of human T cell epitopes in the final composite sequence.

As in the case of antibodies, the invention includes the option of further modifications to the composite protein sequences by random, semi-random or directed mutagenesis of the composite protein to achieve further improvement in one or more other properties of the final protein. It will be understood that the invention is particularly suitable to producing proteins with low immunogenicity when used in humans or used by humans such as proteins for pharmaceutical use, or proteins for use in food, detergents, cosmetics and other consumer items where allergic responses are limited or eliminated by use of compositions of the present invention. It will be understood that the invention is particularly suitable to producing proteins with low allergenicity in humans especially by producing proteins with allergy associated T cell epitopes removed or replaced by non-allergy associated epitopes (e.g. TH2 for TH1 T cell-inducing epitopes) and/or by addition of Tr epitopes to suppress immune responses in allergic individuals. It will be understood that the invention is particularly suitable to producing proteins with reduced inflammatory properties in humans especially by producing proteins with inflammation associated T cell epitopes removed or replaced by non-inflammation associated epitopes (e.g. TH1 for TH2 T cell-inducing epitopes) and/or by addition of Tr epitopes to suppress inflammatory responses.

As discussed herein, the modified/composite proteins and antibodies of the invention are useful in treating disease and exhibit less immunogenicity. Thus, in yet a further aspect, the present invention provides a pharmaceutical formulation comprising a modified antibody, antigen-binding fragment or protein as defined in any one of claims 1 to 18, optionally together with one or more pharmaceutically acceptable excipients, carriers or diluents.

The compositions of the invention may be presented in unit dose forms containing a predetermined amount of each active ingredient per dose. Such a unit may be adapted to provide 5-100 mg/day of the compound, preferably either 5-15 mg/day, 10-30 mg/day, 25-50 mg/day 40-80 mg/day or 60-100 mg/day. For compounds of formula I, doses in the range 100-1000 mg/day are provided, preferably either 100-400 mg/day, 300-600 mg/day or 500-1000 mg/day. Such doses can be provided in a single dose or as a number of discrete doses. The ultimate dose will of course depend on the condition being treated, the route of administration and the age, weight and condition of the patient and will be at the doctor's discretion.

The compositions of the invention may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For applications to the eye or other external tissues, for example the mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administration to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists which may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Preferred unit dosage formulations are those containing a daily dose or sub-dose, as herein above recited, or an appropriate fraction thereof, of an active ingredient.

It should be understood that in addition to the ingredients particularly mentioned above, the formulations may also include other agents conventional in the art having regard to the type of formulation in question, for example those suitable for oral administration may include flavouring agents.

The following examples should not be considered limiting for the scope of the invention. The figures and tables relate to the examples below and are as follows;

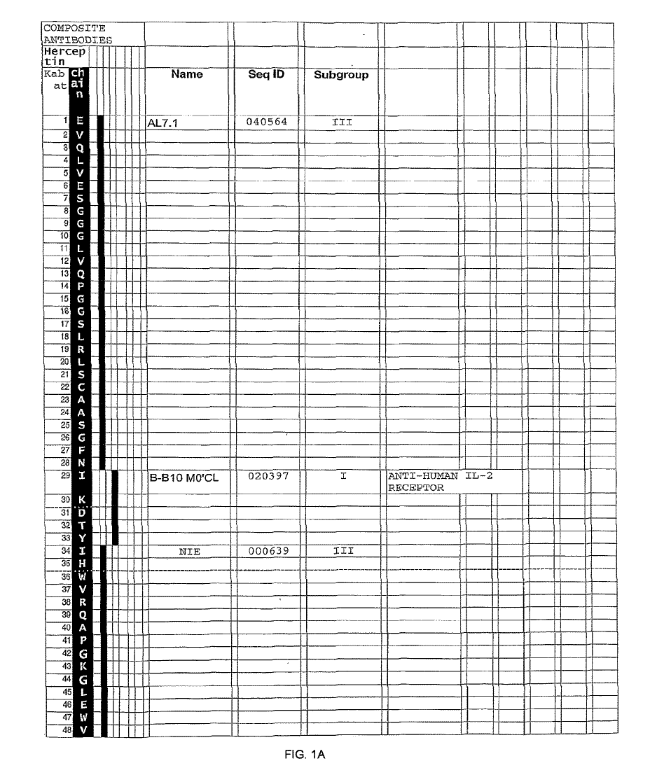

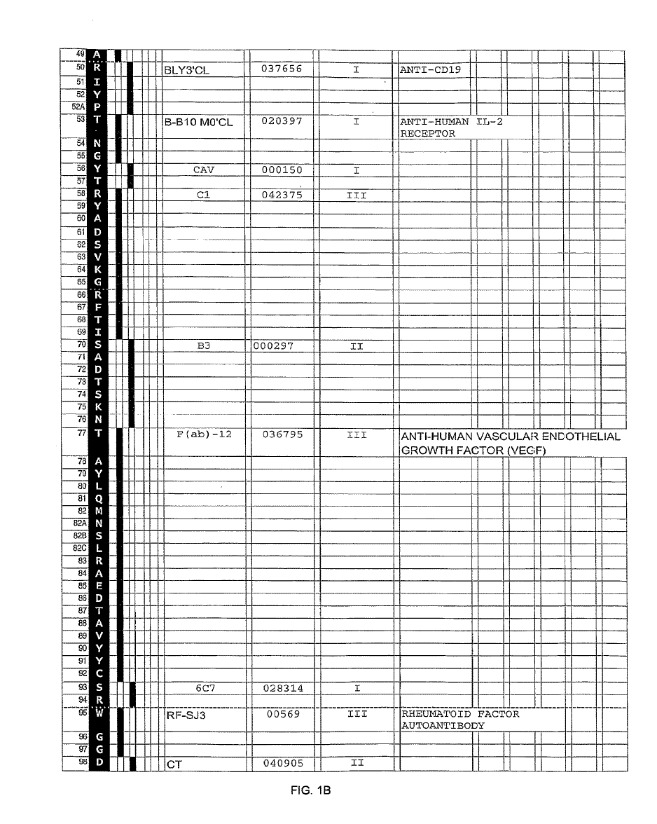

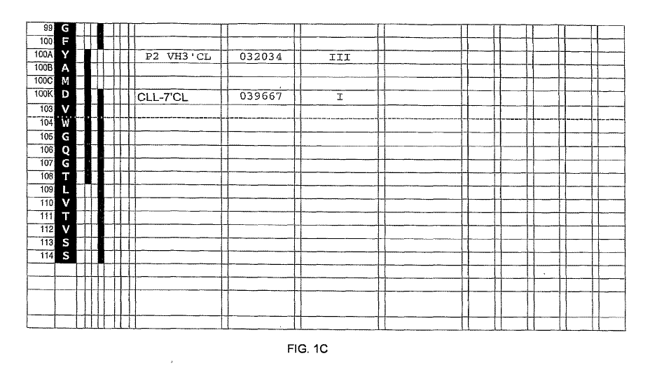

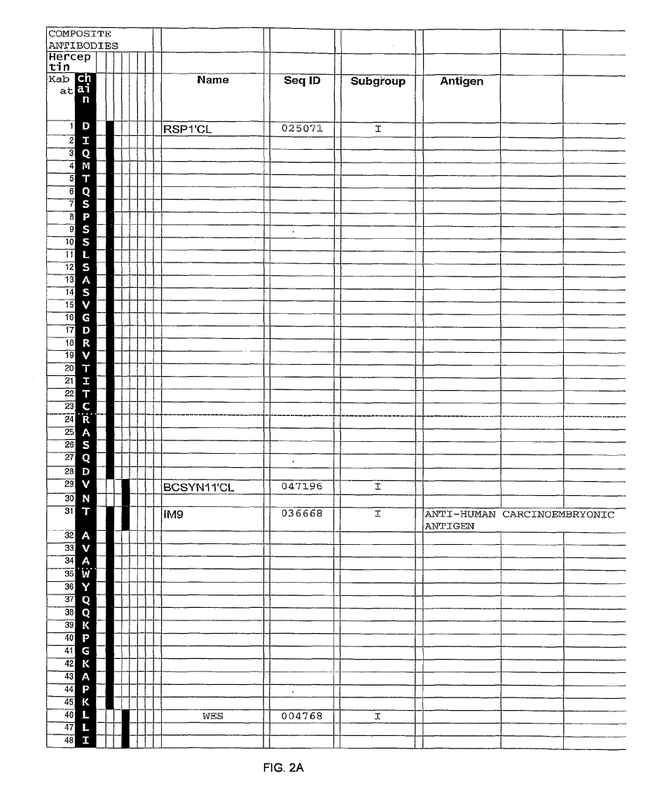

FIGS. 1/2—Sequence of VH (FIG. 1) and VL (FIG. 2) genes used for Composite Human anti-HER2 antibody

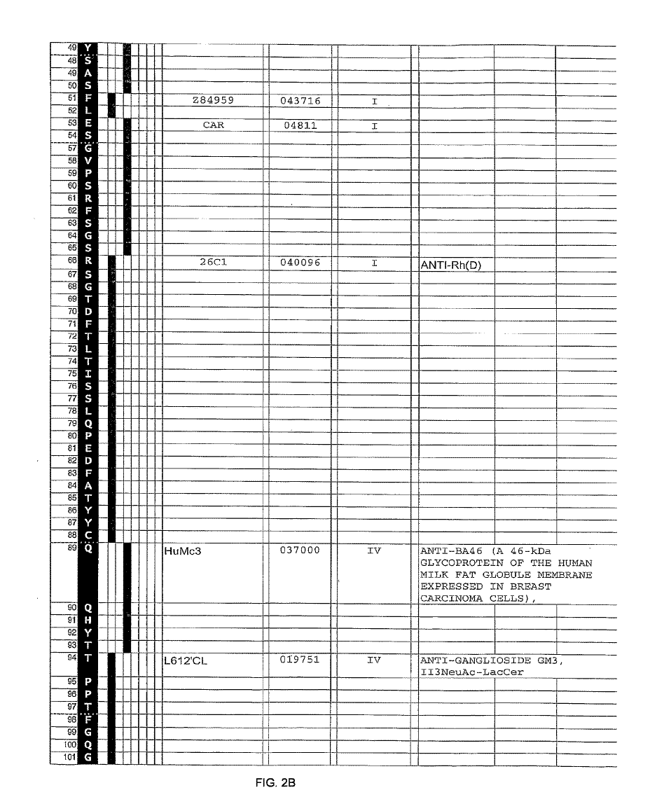

FIG. 3—Inhibition of proliferation of human SK-BR-3 cells after 8 days incubation with chimeric 4D5 IgG1/kappa, Composite Human anti-HER2 antibody and epitope avoided anti-HER2 “EACHAB” with chimeric anti-IgE control (see example 4)

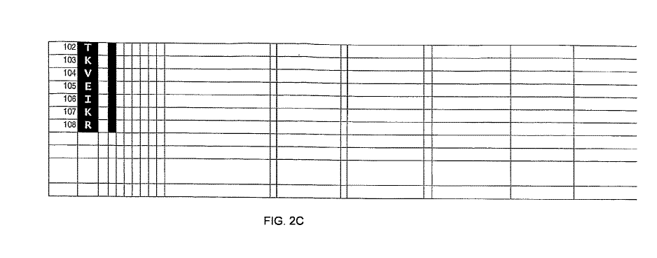

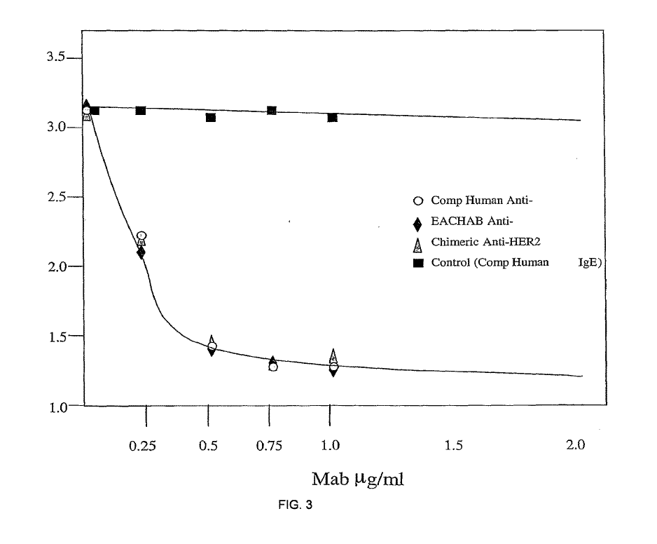

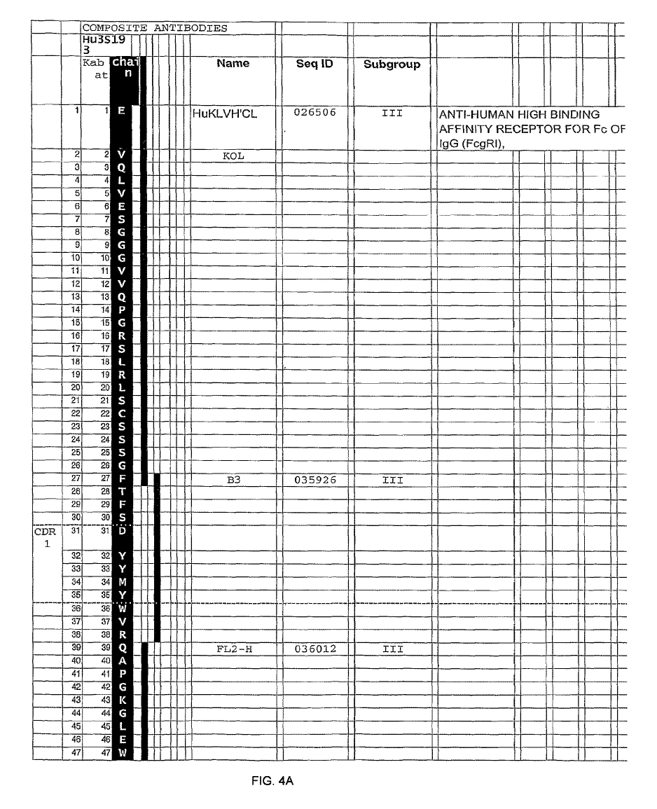

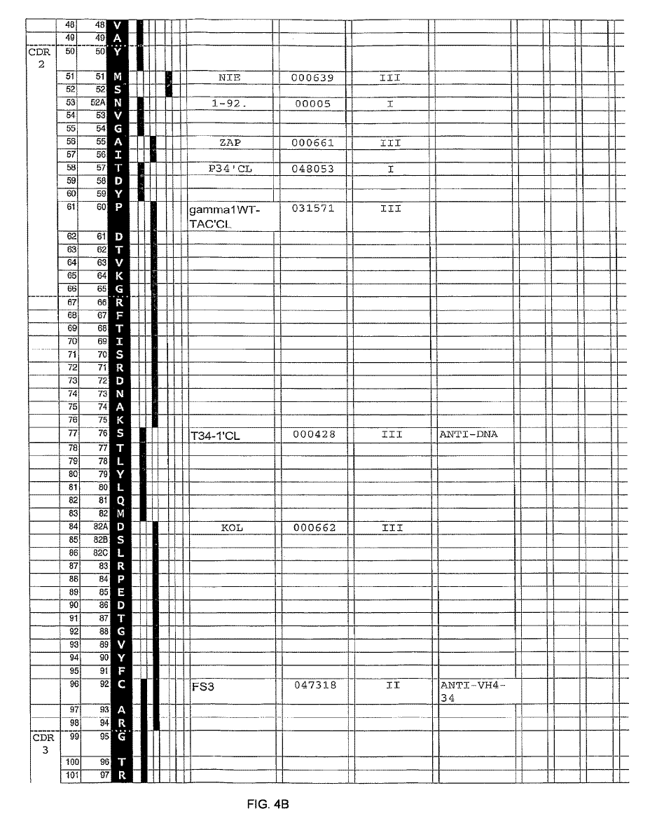

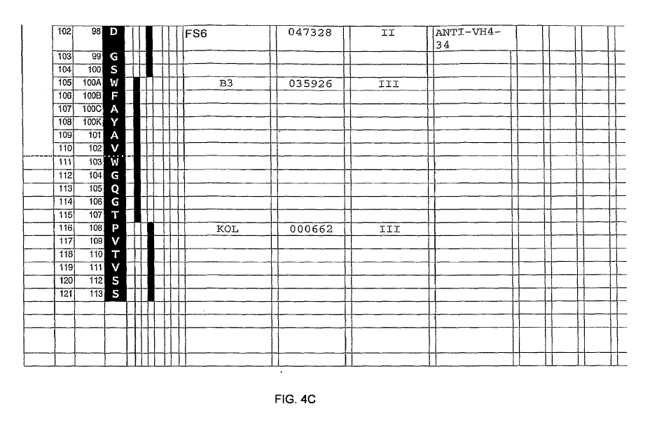

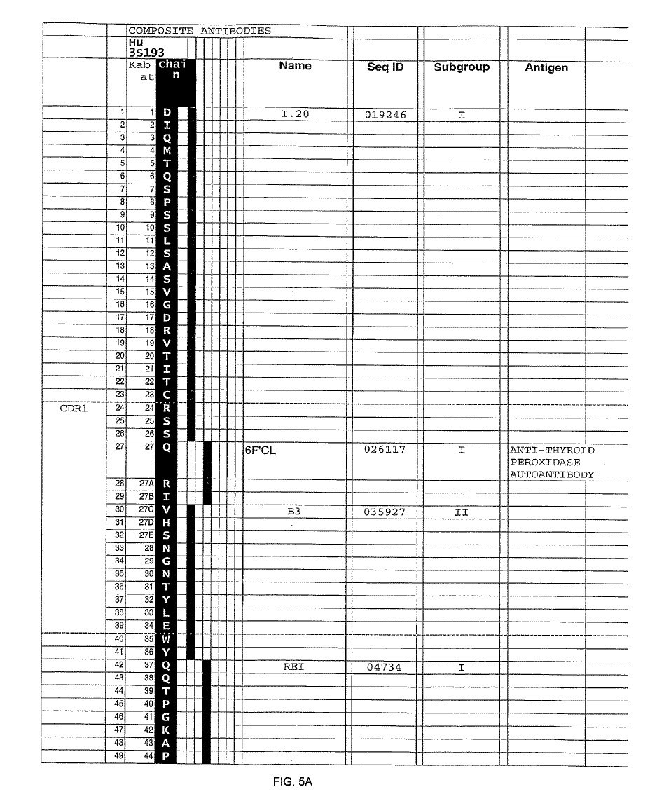

FIGS. 4/5—Sequence of VH (FIG. 4) and VL (FIG. 5) genes used for Composite Human anti-Lewis Y antibody

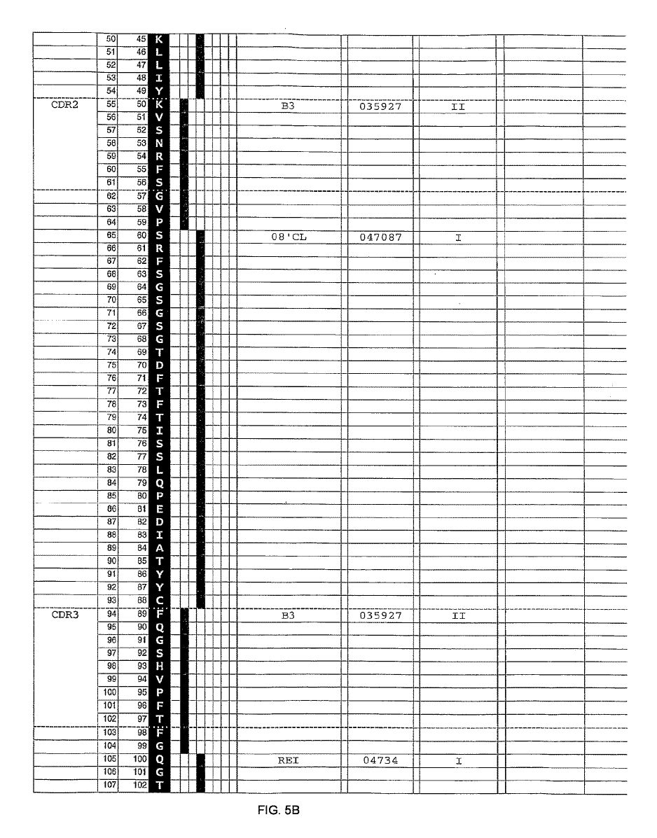

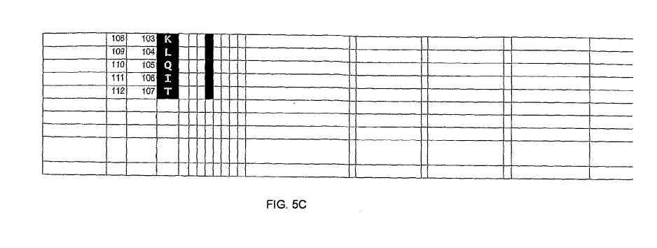

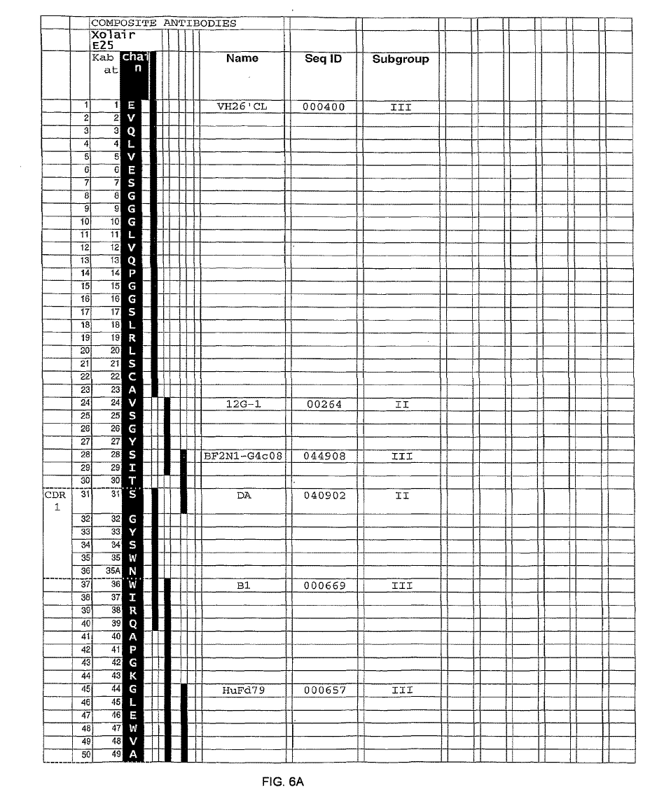

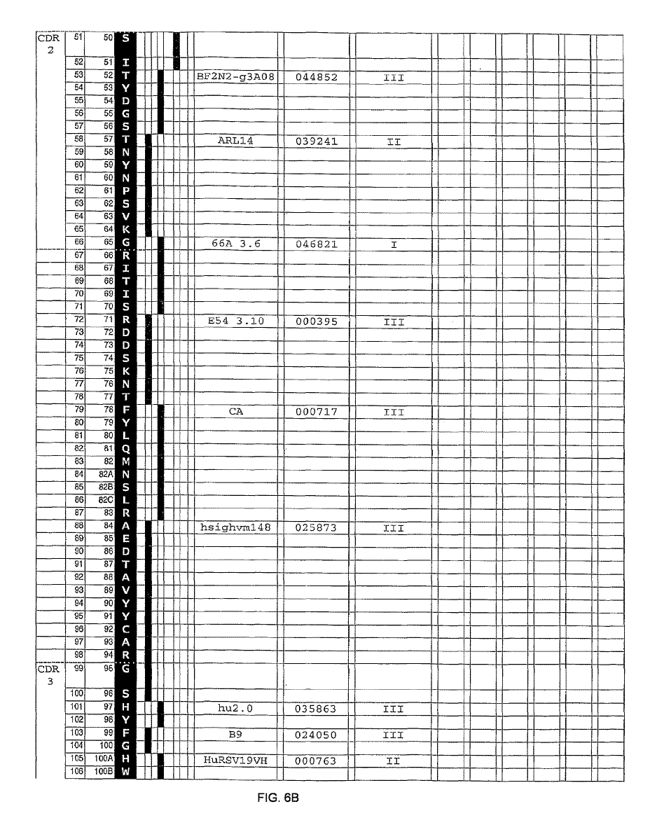

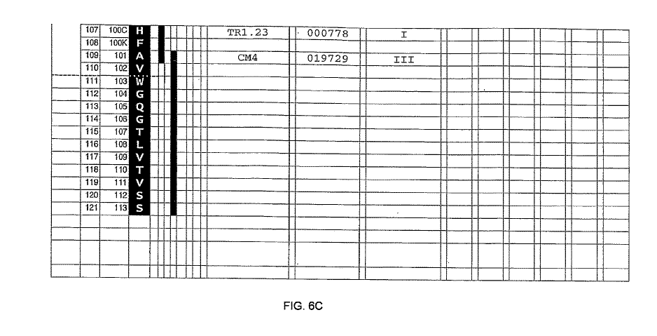

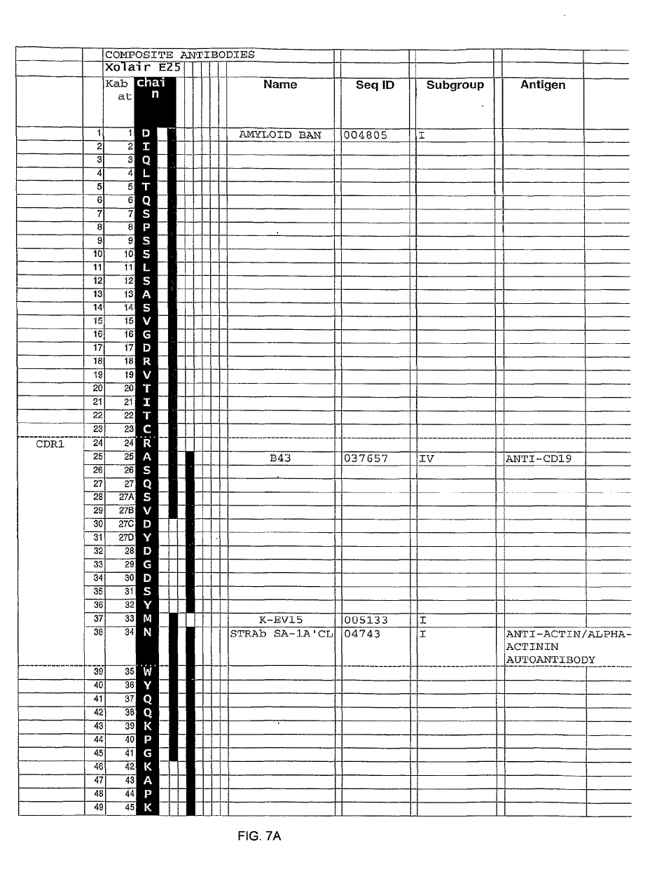

FIGS. 6/7—Sequence of VH (FIG. 6) and VL (FIG. 7) genes used for Composite Human anti-human IgE antibody

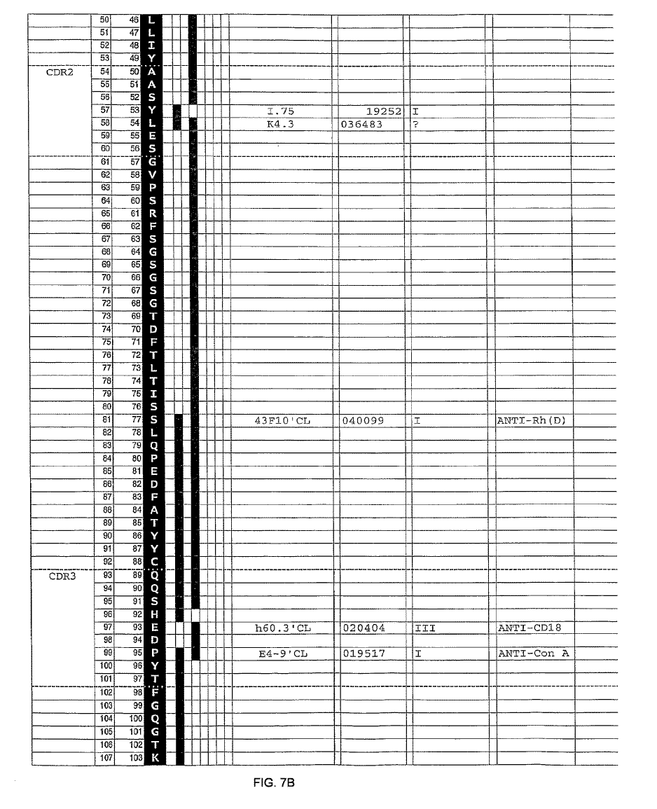

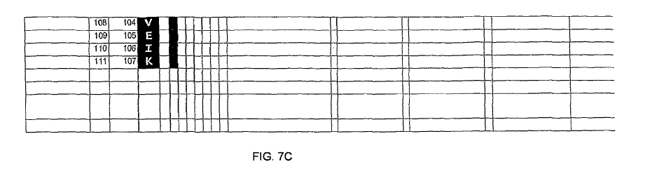

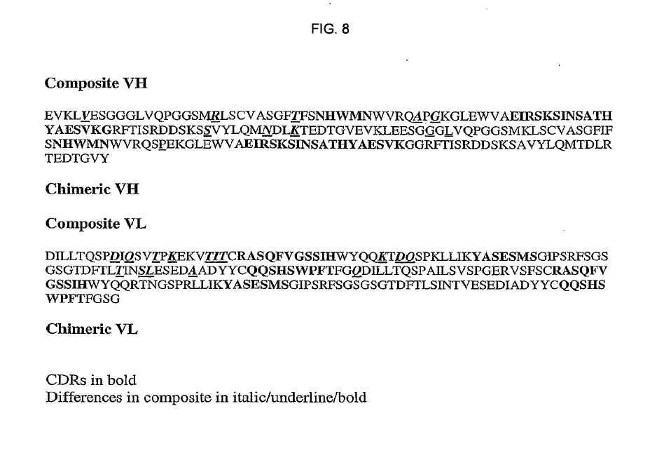

FIG. 8—Sequence of VH and VL genes used for Composite Mouse anti-human TNFα antibody including avoidance of human T cell epitopes

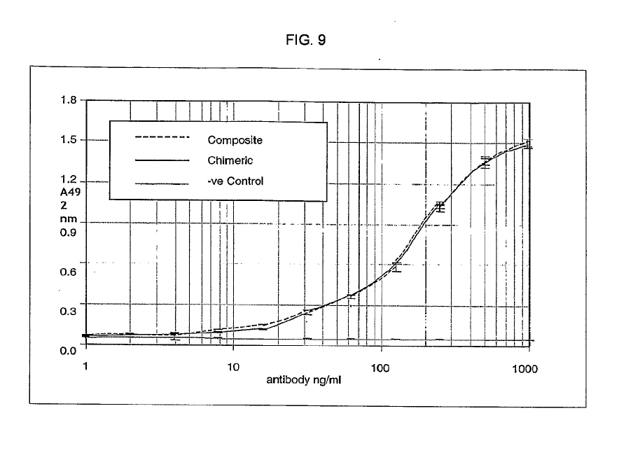

FIG. 9—ELISA for binding to human TNFα by Composite Mouse and chimeric anti-human TNFα antibody

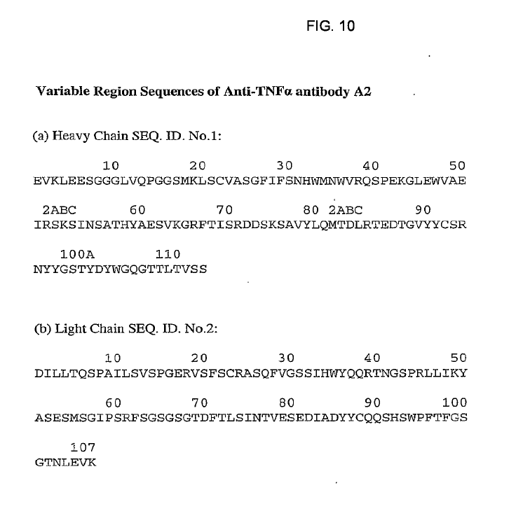

FIG. 10—V region sequences of anti-TNFα antibody A2

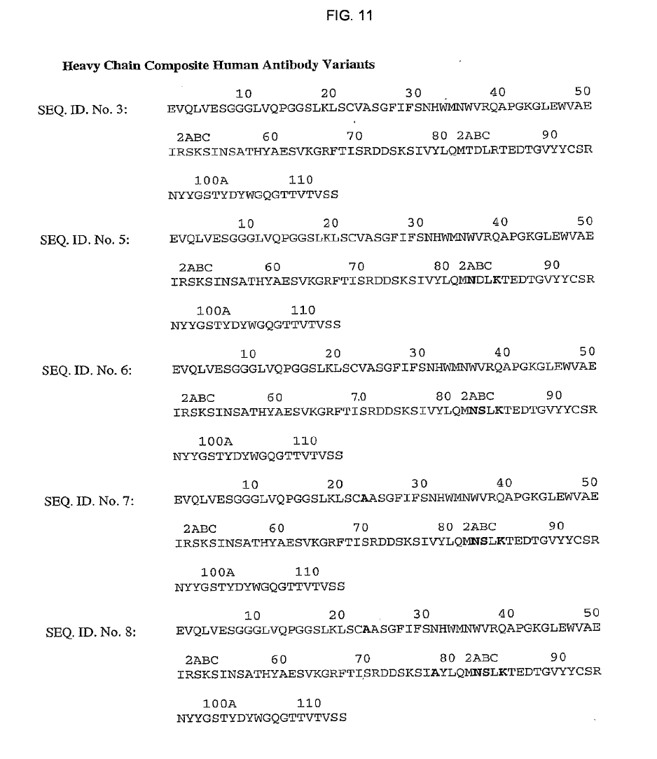

FIG. 11—Sequences of composite human anti-TNFα VH variants

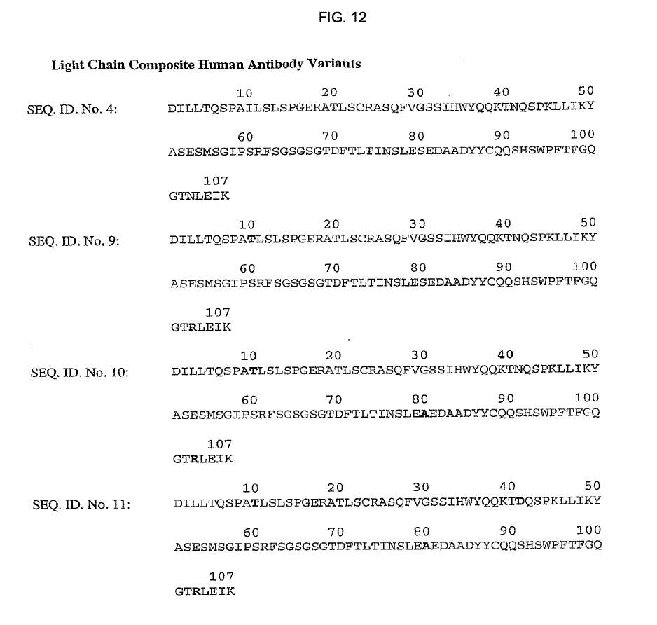

FIG. 12—Sequences of composite human anti-TNFα VL variants

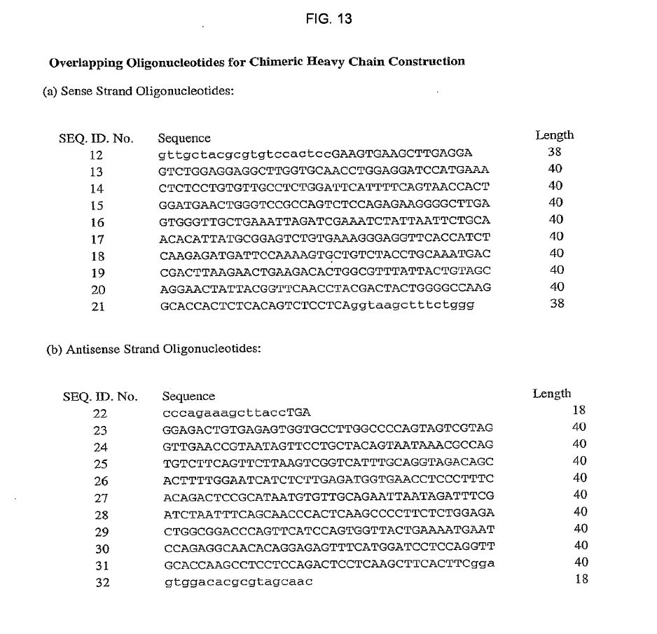

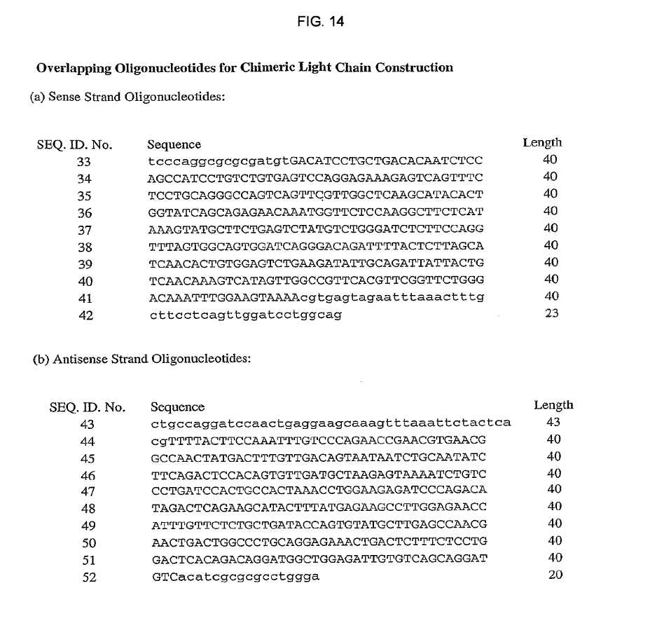

FIGS. 13/14—Oligonucleotides for construction of chimeric mouse:human anti-TNFα VH (FIG. 13) and VL (FIG. 14)

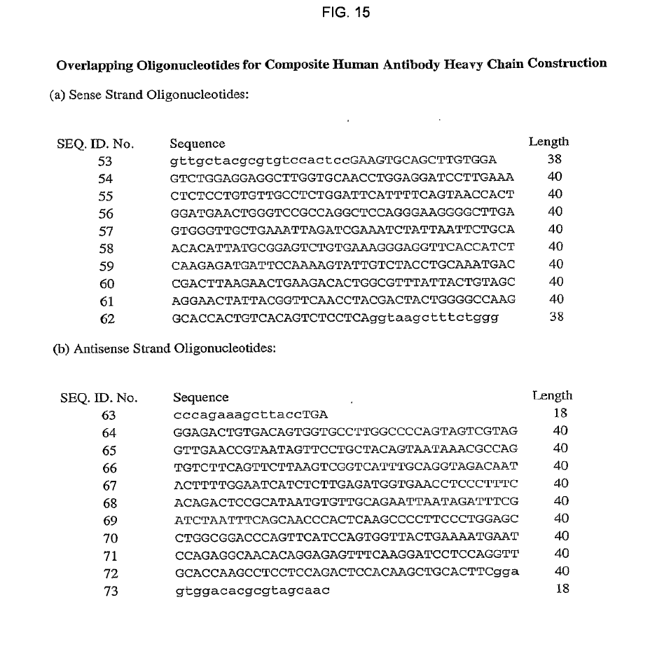

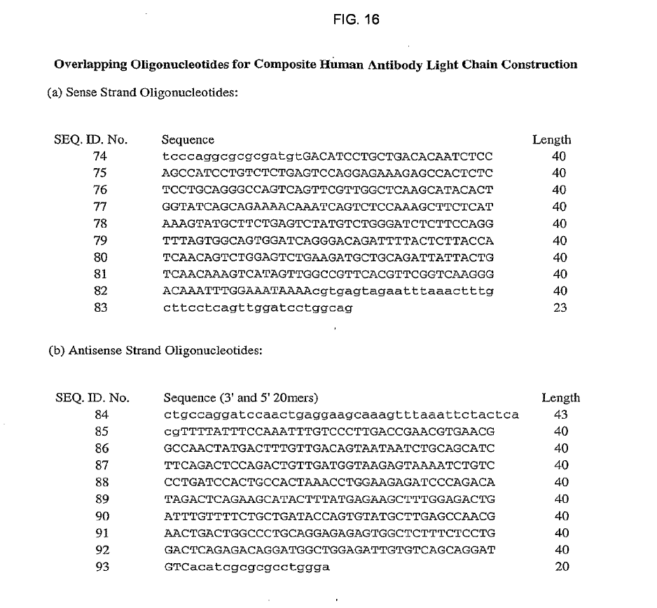

FIGS. 15/16—Oligonucleotides for construction of primary composite human anti-TNFα VII (FIG. 15; corresponding to SEQ ID No. 3 FIG. 11) and VL (FIG. 16; corresponding to SEQ ID No. 4 FIG. 12)

FIG. 17—Oligonucleotides for construction of secondary composite human anti-TNFα VH and VL variants

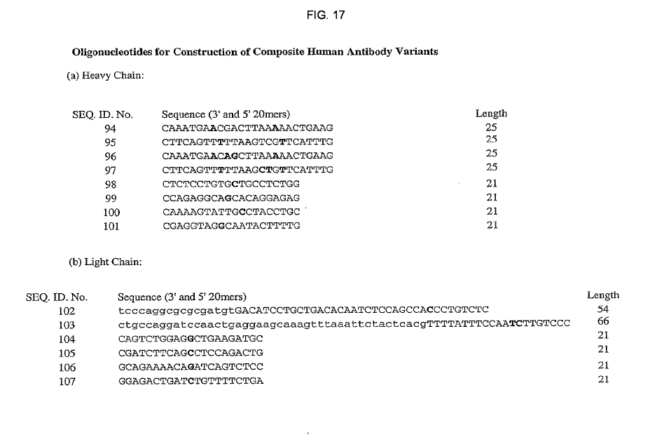

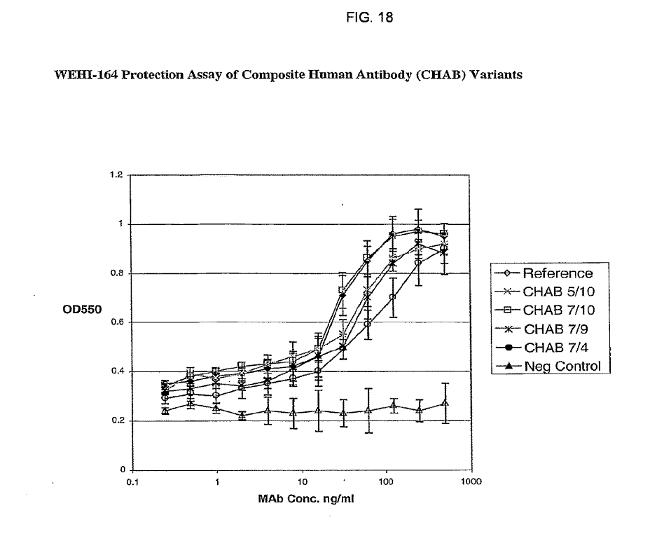

FIG. 18—WEHI-164 protection Assay for composite human anti-TNFα antibodies

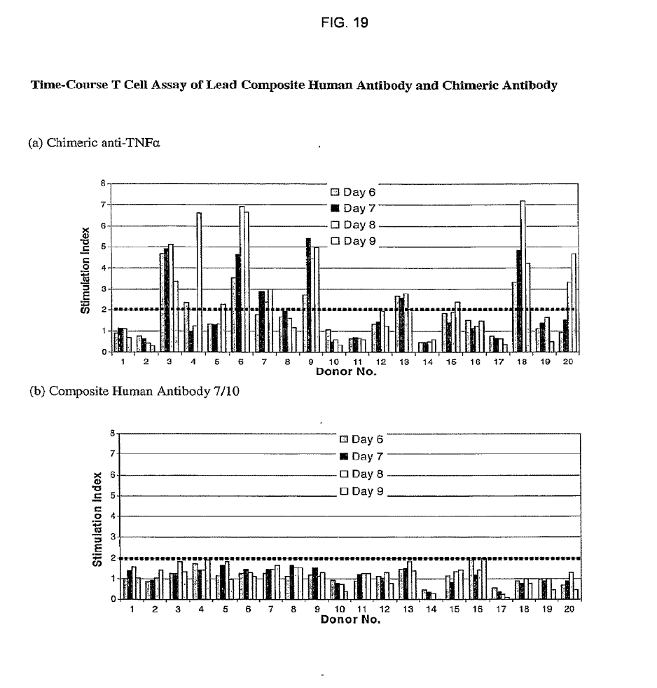

FIG. 19—Time-Course human T cell assay of lead composite human anti-TNFα

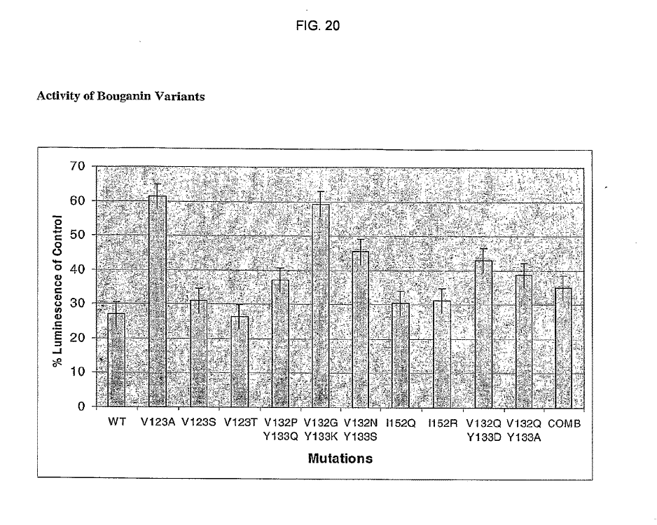

FIG. 20—Activity of composite bouganin molecules with inserted human sequence segments

Tables 1-3—CDRs used in Composite Human antibody scFv library comprising 186×9 residue-long VH CDR3s (table 1), 77×8 residue-long VL CDR3s (table 2), and 153×10 residue-long VL CDR3s (table 3).

Table 4—Human sequence segments used for primary composite human anti-TNFα VH and VL variants

Table 5—Activity of composite human anti-TNFα variants

Table 6—Immunogenic peptide sequences of bouganin and replacement human segments in bouganin variants

### EXAMPLE 1

### Construction of Composite Human Anti-HER2 Antibody

For creation of a human variable region sequence segment library, amino acid sequences from a range of human immunoglobulins were collected into a single database comprising the in silico human variable region sequence library including heavy (VH) and light (VL) chain variable region sequences. Sources of sequences included the NCBI Igblast database (www.ncbi.nih.gov), Kabat databases (Kabat et al., Sequences of Proteins of Immunological Interest, NIH publication 91-3242, 5^{th} ed. (1991) (and later updates)), Vbase (www.mrc-cpe.cam.ac.uk/imt.doc), Genbank (Benson et al., Nucl. Acids Res. 25 (1997) p1-6 or via www.bioinf.org.uk/abs) databases. The reference antibody variable region sequences used was a humanised anti-HER2 antibody known as Herceptin® (Carter et al., Proc. Nat. Acad. Sci. USA, vol 89 (1992) p4285, U.S. Pat. No. 5,821,337). Segments from the in silico human variable region sequence library were selected for identity to the corresponding amino acids in the Herceptin® variable region sequence and combined to produce the composite human VH and VL sequences as shown in FIGS. 1 and 2 respectively.

Recombinant DNA techniques were performed using methods well known in the art and, as appropriate, supplier instructions for use of enzymes used in these methods. Sources of general methods included Molecular Cloning, A Laboratory Manual, 3^{rd} edition, vols 1-3, eds. Sambrook and Russel (2001) Cold Spring Harbor Laboratory Press, and Current Protocols in Molecular Biology, ed. Ausubel, John Wiley and Sons. Detailed laboratory methods are also described in example 7 below. Composite human VH and VL sequences corresponding to Herceptin® were created using, for each chain, eight synthetic oligonucleotides of 30-60 amino acids in length encoding the entire composite human VH and VL sequences. In parallel, as a control reagent, a chimeric form of the mouse monoclonal antibody 4D5 (Hudziak et al., Mol. Cell. Biol., (March 1989) p1165-1172)), was also created using eight synthetic oligonucleotides per chain. Separate VH and VL oligonucleotides were first phosphorylated, mixed at equal molar ratios, heated to 94oC for 5 min in a thermal cycler followed by cooling to 65oC and incubation at 65oC for 2 min. Incubations were then continued at 45oC for 2 min., 35oC for 2 min., 25oC for 2 min and 4oC for 30 min. Oligonucleotides were then ligated using T4 DNA ligase (Life Technologies, Paisley UK) at 14oC for 18 hours.

To each of the VH and VL oligonucleotide mixtures, additional oligonucleotides encoding a 5′ flanking sequence, including a Kozak sequence, the leader signal peptide sequence and the leader intron, and 3′ flanking sequence, including the splice site and intron sequence, were added and annealed as above. The Composite Human V_{H} and V_{K} and the 4D5 expression cassettes produced were cloned as HindIII to BamHI fragments into the plasmid vector pUC19 and the entire DNA sequence was confirmed. These were transferred to the expression vectors pSVgpt and pSVhyg which include human IgG1 (V_{H}) or Kappa (V_{K}) constant regions respectively and markers for selection in mammalian cells. The DNA sequence was confirmed to be correct for the Composite Human V_{H} and V_{K} and 4D5 V_{H} and V_{K} in the expression vectors.

The host cell line for antibody expression was NS0, a non-immunoglobulin producing mouse myeloma, obtained from the European Collection of Animal Cell Cultures, Porton, UK (ECACC No 85110503). The heavy and light chain expression vectors were co-transfected into NS0 cells by electroporation. Colonies expressing the gpt gene were selected in Dulbecco's Modified Eagle's Medium (DMEM) supplemented with 10% foetal bovine serum, 0.8 μg/ml mycophenolic acid and 250 μg/ml xanthine. Transfected cell clones were screened for production of human antibody by ELISA for human IgG. Cell lines secreting antibody were expanded and the highest producers selected and frozen down in liquid nitrogen. The modified antibodies were purified using Prosep®-A (Bioprocessing Ltd, Northumberland, UK). The concentration was determined by ELISA for human IgGK antibody.

The Composite Human antibody and chimeric 4D5 antibodies were tested for inhibition of proliferation of the HER2+ human breast tumour cell line SK-BR-3 in conjunction with a negative control non-Her-2 binding human IgG1/Kappa antibody exactly as described by Hudziak et al. (ibid). The results (FIG. 3) show that Composite Human antibody and the chimeric 4D5 antibodies have equivalent potency in inhibiting growth of SK-BR-3 cells. FIG. 3 also shows data for an alternative “epitope avoided” Composite Human antibody produced as below.

In order to test the epitope avoidance option in the invention, the sequences of the Composite Human heavy and light chain variable regions were analysed for non-self human MHC class II binders using Peptide Threading (www.csd.abdn.ac.uk/˜gjlk/MHC-thread). This software predicts favourable interactions between amino acid side chains of the peptide and specific binding pockets within the MHC class II binding groove. All overlapping 13 mers from the Composite Human heavy and light chain variable sequences were threaded through a database of MHC class II allotypes and scored based on their fit and interactions with the MHC class II molecules. Peptides predicted to bind MHC class II were 13 mers beginning at residues 16 and 67 in VH, and 9 and 44 in VL. As a result, new segments of the human variable region sequence library were chosen instead of those used in the Composite Human sequences of FIG. 1 in order to introduce the amino acid changes VH 18L-A/69I-G; VL 11L-A, 46L-A. A corresponding “epitope avoided” Composite Human antibody (“EACHAB”=Epitope Avoided Composite Human AntiBody) was made by substituting some of the oligonucleotides used to make the antibody corresponding to the sequence in FIG. 1 and the EACHAB was made as in the method described above and tested to show inhibition of SK-BR-5 proliferation equivalent to the standard Composite Human antibody (FIG. 3). This data shows that Composite Human antibodies can be successfully constructed with equal potency to a control chimeric anti-Her-2 antibody and that an EACHAB version of the Composite Human antibody can be generated without loss of potency.

### EXAMPLE 2

### Immunogenicity of Composite Human Anti-HER2 Antibody

T cell proliferation assays were carried out to compare the immunogenicity of the Composite Human anti-HER2 antibody, the EACHAB variant and the chimaeric 4D5 antibody (see example 1). These antibodies were prepared from NS0 cells grown in serum-free, animal derived component-free, protein-free medium, HyClone HyQ®ADCF-Mab™ (Hyclone Cat No: Cat no: SH30349) supplemented with HyQ®LS1000 Lipid Supplement (Hyclone Cat No: SH30554) and sodium pyruvate (Gibco Cat No: 11360-039). After buffer exchange into 50 mM MES pH6 on a Sephadex G25 (PD10 column), the antibodies were each passed through a cation exchange column (Mono-S 10/10) and eluted with a sodium chloride gradient (0 to 0.5M). The antibody containing fractions were then applied to a Superdex 200 preparative column (XK16/60) run in PBS. Peak fractions were pooled and stored at 4° C. The antibody concentrations were determined by ELISA for human IgG.

Immunogenicity analysis was performed using PBMCs (peripheral blood mononuclear cells) that were isolated from healthy human donor blood and cryopreserved in liquid nitrogen. Each donor was tissue-typed using an Allset™ PCR based tissue-typing kit (Dynal, Wirral, UK) and 20 healthy donors were selected according to individual MHC haplotypes. 2 ml bulk cultures containing 4×10⁶ PBMC in AIM V (Invitrogen, Paisley, UK) were incubated in a 24 well tissue-culture plate with test peptides (5 μM final concentration) and proliferation was assessed on days 5, 6, 7, and 8 by gently resuspending the bulk cultures and transferring triplicate 100 μl samples of PBMC to a U-bottomed 96 well plate. Cultures were harvested onto glass fibre filter mats using a Tomtec Mach III plate harvester (Receptor Technologies, UK) and counts per minute (cpm) values determined by scintillation counting using a Wallac Microbeta TriLux plate reader (using a paralux high efficiency counting protocol). For each test antibody, the stimulation index (SI) was calculated as the ratio of counts per minute (cpm) of the test antibody:cpm of the negative control with SI>2 considered a significant T cell epitope response. The results showed that the chimaeric 4D5 antibody induced significant proliferative responses on at least one of the four days of proliferation tested (SI greater than 2) in five of twenty healthy donors tested (25%), the Composite Human anti-HER2 antibody induced SI>2 in three of twenty donors (15%) whilst the EACHAB anti-HER2 antibody induced SI>2 in none of twenty donors (0%). These results indicated an order of immunogenicity of chimeric 4D5>Composite Human anti-HER2>EACHAB anti-HER2 with the latter showing no evidence of immunogenicity in any donor blood sample tested.

### EXAMPLE 3

### Construction of Composite Human Anti-Lewis Y Antibody

A Composite Human antibody specific for sialylated Lewis Y antigen was constructed as described in example 1 using, as the reference antibody variable region sequences, the humanised 3S193 antibody (Scott et al.; Cancer Res., 60 (2000) p3254-3261, U.S. Pat. No. 5,874,060). Segments from the in silico human variable region sequence library were selected for identity to the corresponding amino acids in the humanised 3S193 variable region sequence and combined to produce the Composite Human VH and VL sequences as shown in FIGS. 4 and 5 respectively. In parallel, a reference chimeric anti-Lewis Y antibody was made from the reference V region sequences. Human IgG1 (V_{H}) and Kappa (V_{K}) constant regions were used on both the Composite Human anti-Lewis Y antibody and the chimeric reference antibody and antibodies were tested by ELISA against synthetic Lewis Y-HSA conjugate as described in U.S. Pat. No. 5,874,060. The data showed a minimum concentration of 0.1 ug/ml chimeric antibody to give a binding signal in the assay compared to 0.15 ug/ml Composite Human antibody which is consistent with the data of U.S. Pat. No. 5,874,060.

### EXAMPLE 4

### Construction of Composite Human Anti-IgE Antibody

A Composite Human Anti-IgE antibody was constructed as described in example 1 using, as the reference antibody variable region sequences, the humanised anti-IgE antibody known as Xolair® (Presta et al., J. Immunol., 151(5) (1993) p2623-2632). Segments from the in silico human variable region sequence library were selected for identity to the corresponding amino acids in the Xolair® variable region sequence and combined to produce the Composite Human VH and VL sequences as shown in FIGS. 6 and 7 respectively. In parallel, a reference chimeric anti-IgE antibody was made from the reference V region sequences. Human IgG1 (V_{H}) and Kappa (V_{K}) constant regions were used on both the Composite Human Anti-IgE antibody and the chimeric reference antibody.

The specificity of the Fabs was further characterized by surface plasmon resonance (BIAcore 2000, Biacore AB, Uppsala, Sweden). Recombinant human IgE Fab was produced as described by Flicker et al., J. Immunol., 165 (2000) p3849-3859. Test antibodies were purified and immobilized onto flow cells of a CM chip using a NHS/EDC kit (Biacore) to obtain 2010 RU for chimeric anti-IgE and 2029 RU for Composite Human anti-IgE. 10 and 25 nM recombinant human IgE Fab in Hepes-buffered saline (10 mM Hepes, 3.4 mM EDTA, 150 mM NaCl, 0.05% (v/v) surfactant P20, pH 7.4) was passed over the test antibodies at a flow rate of 5 μl/min for 10 minutes. The results showed that for both 10 and 25 nM IgE Fab, an equivalent SPR (surface plasmon resonance) curve was detected for the chimeric anti-IgE and the Composite Human anti-IgE antibodies showing that the latter had successfully achieved binding efficiency equivalent to the reference anti-IgE antibody.

### EXAMPLE 5

### Generation and Screening of Composite Human scFv Libraries

The strategy for initial construction of the human scFv (single-chain Fv) library was to construct seven consensus human VH and four consensus human VL (kappa) genes as detailed in Knappik et al., J. Mol. Biol., 296 (2000) 57-86 and to clone into these a large number of VH and VL CDR3 segments from databases of human variable regions. This list of CDR3s is shown in table 1 for VH CDR3s, table 2 for VL CDR3s of 8 amino acids and table 3 for VL CDR3s of 10 amino acids. For the master VH and VL construction, 6 overlapping synthetic oligonucleotides encoding VH and VL up to the end of framework 3 were synthesised as detailed by Knappik et al., ibid, and subjected to recursive PCR (Prodromou and Pearl, Protein Engineering, 5 (1992) 827-829). These were ligated into EcoRV digested pZero-1 vector (Invitrogen, Paisley, UK). For addition of CH1 and C kappa, both initially with 4D5 CDR3s (Carter et al, Bio/Technology, 10 (1992) 163-167), the protocol of Knappik et al., ibid, was followed except that the VH-CH1 SapI-EcoRI and VL-C kappa NsiI-SphI fragments were both blunt-end cloned into EcoRV digested pZero-1.

TABLE 1

| # Name | H3 | Length-H3 | Subgroup (H) |
|---|---|---|---|
| MUC1-1′CL | DFLSGYLDY | 9 | I |
| ALL1-1′CL | VRGSGSFDY | 9 | III |
| ALL7-1′CL | DRGGNYFDY | 9 | III |
| L36′CL | MYNWNFFDY | 9 | I |
| 5.M13′CL | AGLGMIFDY | 9 | I |
| Au2.1′CL | RGFNGQLIF | 9 | I |
| M71′CL | ALTGDAFDI | 9 | II |
| VH6.N1′CL | TKLDWYFDL | 9 | II |
| E55 6.X′CL | RYGGFYFDY | 9 | II |
| E55 6.11′CL | GYSNEGMDV | 9 | II |
| VH6.A5′CL | SWDGYSYIY | 9 | II |
| VH6-EX8′CL | QMGAEYFQH | 9 | III |
| E54 4.2′CL | DMSLDAFDI | 9 | II |
| RF-SJ4′CL | GSVGATLGE | 9 | II |
| 3.A290′CL | YGDYHYFDY | 9 | III |
| A95 | GVGSSGWDH | 9 | III |
| 60P2′CL | KGSLYYFDY | 9 | III |
| E55 3.6′CL | PNWNDAFDI | 9 | III |
| E55 3.16′CL | RGIPHAFDI | 9 | III |
| 333′CL | PPEVESLRS | 9 | III |
| 1H1′CL | PPEVESLRS | 9 | III |
| 126′CL | PPEVESLRS | 9 | III |
| 1B11′CL | PPEVESLRS | 9 | III |
| 115′CL | PPEVESLRS | 9 | III |
| 112′CL | PPEVESLRS | 9 | III |
| 2C12′CL | PPEVESLRS | 9 | III |
| 2A12′CL | PPEVESLRS | 9 | III |
| BUT | DLAAARLF? | 9 | III |
| KOL-based | QGTIAGIRH | 9 | III |
| resh. | | | |
| CAMPATH-9 | | | |
| L2E8′CL | EDYYYGMDV | 9 | III |
| s5D4′CL | DPINWYFDL | 9 | III |
| ss4′CL | DRAAGDRDY | 9 | III |
| P2-57′CL | HQMYSNSDY | 9 | I |
| HuHMFG1′CL | SYDFAWFAY | 9 | I |
| NEW-based | QGTIAGIRH | 9 | II |
| resh. | | | |
| CAMPATH-9 | | | |
| TR1.10′CL | VLGIIAADH | 9 | I |
| L3B2′CL | DLTGDAFDI | 9 | I |
| DAW | SCGSQYFDY | 9 | II |
| ss7′CL | LWNWDAFDI | 9 | I |
| ss6′CL | DIMTWGFDY | 9 | I |
| s5A9′CL | SNWYWYFDL | 9 | III |
| NEWM | NLIAGCIDV | 9 | II |
| L2A12′CL | GGKGGEFDD | 9 | I |
| B5G10H′CL | DSGNYRIDY | 9 | II |
| E55 3.9′CL | DPRLDAFDI | 9 | III |
| SpA1-29′CL | GYSYPVWGR | 9 | III |
| AM28′CL | LVGNSWLDY | 9 | III |
| BM2′CL | DL?GLVVEY | 9 | III |
| CM29′CL | KVSLSAFDI | 9 | III |
| B-B10 M0′CL | RGDAMYFDV | 9 | I |
| HSVCBM8′CL | DPNPWYFDL | 9 | III |
| HSVCD53′CL | DYGDYAFDI | 9 | III |
| HSVCBG6′CL | SAHSDAFDM | 9 | III |
| MICA 4′CL | LEGLGWFDP | 9 | I |
| 1/11′CL | RSDYGAIDY | 9 | III |
| 5/8′CL | NLGFYHMDV | 9 | III |
| B6204′CL | EARGGGGEY | 9 | III |
| VH CLONE | EGWISALNG | 9 | III |
| 1′CL | | | |
| VH CLONE | EGEGEYFDY | 9 | III |
| 32′CL | | | |
| MG6-1′CL | ERTSGDFDF | 9 | III |
| MG6-3′CL | NSPGATFES | 9 | III |
| Daudi′CL | GNGQKCFDY | 9 | III |
| IE4′CL | RGSLQYLDY | 9 | I |
| IF10′CL | NNGSYYFDY | 9 | I |
| hsighvm148′CL | GSDYSNFAY | 9 | III |
| E3-MPO′CL | STHRSAFDV | 9 | II |
| rev9Fd′CL | EGVHKNFDH | 9 | III |
| NANUC-1′CL | LSRAGGFDI | 9 | III |
| Patient | RMPAVAFDY | 9 | II |
| 14′CL | | | |
| 14G1′CL | RMPAVAFDY | 9 | II |
| 14G2′CL | RMRAVAFDY | 9 | II |
| 14G3′CL | RMPAVAFDY | 9 | II |
| A15′CL | DYGGNPAEL | 9 | I |
| G15′CL | GPTCSGGSC | 9 | I |
| M11′CL | RKGAAHFDY | 9 | I |
| RF-DI1′CL | EEVGGYFQH | 9 | III |
| AC-18′CL | DFDGGSFDY | 9 | III |
| AC-29′CL | DFDGGSLDY | 9 | III |
| AC-40′CL | DFDGGSFDY | 9 | III |
| TR35′CL | KVPSHGMDY | 9 | III |
| TR36′CL | KVPSHGMDY | 9 | III |
| TR37′CL | KVPSHGMDY | 9 | III |
| TR38′CL | KVPSHGMDY | 9 | III |
| L34′CL | QPLARHFDP | 9 | III |
| L100′CL | GPLMRWFDD | 9 | III |
| WG1′CL | VAVAGGFDP | 9 | III |
| RF-ET5′CL | GVEVAGTAS | 9 | I |
| RF-ET10′CL | YYESSAGPP | 9 | III |
| EW-D1′CL | EIPRGGSCY | 9 | III |
| EW-D3′CL | EIPRGGSCY | 9 | III |
| KN-D6′CL | KEKWDSSRC | 9 | III |
| HH-M2′CL | GSAAAGTQG | 9 | III |
| AK-D8′CL | DFSWAGPHF | 9 | III |
| BALL-1′CL | GTHYYDIRV | 9 | III |
| YJ | DGSGSYEGN | 9 | III |
| K2.2 | GGAVAAFDY | 9 | III |
| E2.5 | KPVTGGEDY | 9 | III |
| MSL5 | DYDGAWFAY | 9 | I |
| Hb-2 | WDGRLLVDY | 9 | III |
| b4′CL | HKGLRYFDY | 9 | III |
| b3′CL | HKGLRYFDY | 9 | III |
| b2′CL | HKGLRYFDY | 9 | III |
| b5′CL | HKGLRYFDY | 9 | III |
| b17′CL | HKGLRYFDY | 9 | III |
| b19′CL | HKGLRYFDY | 9 | III |
| A3-H2′CL | YRGDTYDYS | 9 | III |
| A3-M9′CL | WVGATTSDY | 9 | III |
| Tmu69′CL | EDMDYGMDV | 9 | III |
| Amu1d4-3′CL | GGRDRYLVY | 9 | III |
| 1946′CL | VRVSYGMDV | 9 | V |
| GN901v1.0 | MRKGYAMDY | 9 | III |
| GN901v1.1 | MRKGYAMDY | 9 | III |
| N901H/KOL | MRKGYAMDY | 9 | III |
| N901H/G36005 | MRKGYAMDY | 9 | III |
| N901H/PLO123 | MRKGYAMDY | 9 | III |
| Patient 14′CL | RMPAVAFDY | 9 | II |
| 14G1(2)′CL | RMPAVAFDY | 9 | II |
| 14G2′CL | RMRAVAFDY | 9 | II |
| 14G3′CL | RMPAVAFDY | 9 | II |
| CLL-8′CL | TSIVRGFGP | 9 | II |
| BA-1F′CL | DFFRDYFDY | 9 | I |
| BA-2P′CL | DFFRDYFDY | 9 | III |
| L3055 4.6′CL | GGTQPFDIR | 9 | II |
| 15′CL | SQASGPFDY | 9 | I |
| CL-G′CL | GLYQLLFDY | 9 | III |
| CL-O′CL | AGGRTSFDP | 9 | I |
| BA3′CL | EGNTKAPDY | 9 | III |
| PS′CL | NGTSGDFDY | 9 | II |
| HNK20 hu7 | YGTSYWFPY | 9 | I |
| HNK20 hu10 | YGTSYWFPY | 9 | I |
| Amu1d4-3′CL | GGRDRYLVY | 9 | III |
| Amu1e10-3′CL | LRYQLLYNY | 9 | I |
| 1e8-3′CL | YIAYDAFDI | 9 | I |
| 1f7-3′CL | ITPRNAVDI | 9 | III |
| Agamma41-3′CL | DGLLAATDY | 9 | III |
| Agamma8-3′CL | DRAYLDFWG | 9 | III |
| Amu10-3′CL | DKEPAYFDY | 9 | I |
| Amu2-1′CL | RGFNGQLIF | 9 | I |
| Amu40-2′CL | LSVVVPAAL | 9 | III |
| Amu70-1′CL | LADDDPEDF | 9 | I |
| Tmu69′CL | EDMDYGMDV | 9 | III |
| B7-g2B01′CL | SAGGSAWST | 9 | III |
| B8-g3C11′CL | DRSYYGMDV | 9 | III |
| B8-g3F05′CL | DKGTRYSDQ | 9 | III |
| BF1N-g3C12′CL | WLVEGSFDY | 9 | III |
| BF1N-g3H05′CL | GYVGSSLDY | 9 | III |
| BF1P-g2A11′CL | WHQLRGPDY | 9 | III |
| BF2P1-g3D10′CL | ENSDYYFDY | 9 | III |
| BF2P1-g3E12′CL | DGTYGSGVR | 9 | III |
| BF2P2-g3C10′CL | GGSMVPFDY | 9 | III |
| BF2P2-g3D05′CL | RGWNYYFDS | 9 | III |
| BF2P2-g3D12′CL | DAYYYGLDV | 9 | III |
| BF2P3-g3C10′CL | DGRYDPIDY | 9 | III |
| BF2P1-g7B02′CL | VGSSGWYDY | 9 | I |
| BF2N1-g1C10′CL | DLYDYYDEP | 9 | I |
| BF2N2-g1A11′CL | DGAAASFDY | 9 | I |
| BF2N2-g1E01′CL | VVGADYFDY | 9 | I |
| BF2N1-g3F03′CL | DQNWGYFDY | 9 | III |
| BF2N2-g3B07′CL | GVLRDAFDI | 9 | III |
| BF2N2-g3C03′CL | ASDGYGMDV | 9 | III |
| BF2N2-g3F07′CL | GVLRHALDI | 9 | III |
| BF2N1-g4A03′CL | GGCGWYKNY | 9 | III |
| BF2N1-g4B10′CL | GSNYAKTGY | 9 | III |
| BF2N1-g4C11′CL | GKFQLLFDY | 9 | III |
| BF2N1-g6A07′CL | ALHGGGMDV | 9 | III |
| BF2N1-g6F07′CL | ALHGGGMDV | 9 | II |
| BF2N2-g6D09′CL | VYPPDAFDL | 9 | III |
| mAbRWL1′CL | PWDYWFFDL | 9 | II |
| SV-10 | DRVAAAGDY | 9 | III |
| SV-7 | DKGTRYSDQ | 9 | III |
| SV-9 | DRVATIPDY | 9 | III |
| DN6′CL | ERGITLMDV | 9 | I |
| DN7′CL | ERGITLMDV | 9 | I |
| SC12′CL | LDWLLPIDY | 9 | I |
| SC13′CL | LDWLLPIDY | 9 | I |
| D11′CL | DDGDRAFGY | 9 | III |
| JON′CL | DPWPAAFDI | 9 | III |
| DEZ′CL | VRGSWSGDS | 9 | III |
| BAR | RHSSDWYPY | 9 | III |
| KC13H′CL | SSPYGALDY | 9 | III |
| clone 15′CL | GLDQYKTGH | 9 | II |
| B22′CL | GAGAAPHDY | 9 | III |
| P13′CL | GAGAAPHDY | 9 | III |
| PS′CL | NGTSGDFDY | 9 | II |
| Patient 2 | ALRPATFDF | 9 | III |

TABLE 2

| # Name | L3 | Length-L3 | Class |
|---|---|---|---|
| HIV-s8′CL | QQYADLIT | 8 | IGG1-KAPPA |
| FOG1-A4′CL | QQYYSTPT | 8 | -KAPPA |
| SA-4B′CL | QQYNTYPT | 8 | IGG-KAPPA |
| HIV-b5′CL | QQGNSFPK | 8 | IGG1-KAPPA |
| HIV-loop8′CL | QQYGYSLT | 8 | IGG1-KAPPA |
| Reg-A′CL | QQFGGSFT | 8 | KAPPA |
| 9F12Fab′CL | QQSSNTVT | 8 | KAPPA |
| GP68′CL | QQYNSLIT | 8 | IGG1-KAPPA |
| C471′CL | QQYNNWPT | 8 | IGM-KAPPA |
| B8807′CL | LQHNSYPF | 8 | IGM-KAPPA |
| B122′CL | QQYNSQYT | 8 | IGM-KAPPA |
| B6204′CL | QQYGSLWT | 8 | IGM-KAPPA |
| IM-9′CL | QHYNRPWT | 8 | IGG-KAPPA |
| T48.16-G8′CL | QQYGSRLT | 8 | KAPPA |
| 7F′CL | QHYGTPRT | 8 | IGG1-KAPPA |
| 1A6′CL | QQYNNWPT | 8 | IGM-KAPPA |
| 1.69′CL | MQATQFPT | 8 | IGM-KAPPA |
| antiTac | HQASTYPL | 8 | KAPPA |
| WE | QQYGRSPR | 8 | KAPPA |
| D1.1 | QQDDLPYT | 8 | KAPPA |
| K2.2 | QNDNLPLT | 8 | KAPPA |
| E1.1 | QQESLPLT | 8 | KAPPA |
| E2.4 | QQDNLPLT | 8 | KAPPA |
| E2.5 | QQESLPCG | 8 | KAPPA |
| E2.11 | QQDSLPLT | 8 | KAPPA |
| SSaPB | QQYGSSRS | 8 | IGM-KAPPA |
| SEGaBM | QQYGSSRT | 8 | IGM-KAPPA |
| SELcLN | QQYCGSLS | 8 | IGM-KAPPA |
| mAb5.G3′CL | QQSYSTLT | 8 | IGM |
| P7′CL | QLYGSSLT | 8 | KAPPA |
| PA | QQYNNLWT | 8 | KAPPA |
| CAR | QQYNTFFT | 8 | KAPPA |
| Taykv322′CL | QQYGSSPT | 8 | KAPPA |
| Taykv310′CL | QQYGSSLT | 8 | KAPPA |
| Taykv320′CL | QQYGSSLT | 8 | KAPPA |
| slkv22′CL | QQYGSSKT | 8 | KAPPA |
| LES | QQYNNWPP | 8 | KAPPA |
| RF-TMC1′CL | QHRNNWPP | 8 | IGM-KAPPA- |
| | | | III |
| slkv4′CL | QQRSNWPS | 8 | KAPPA |
| MD3.13′CL | QQYGSSPT | 8 | KAPPA |
| VJI′CL | QQYDTIPT | 8 | KAPPA |
| rsv13L′CL | QASINTPL | 8 | IGG1-KAPPA |
| II.2′CL | MQALQPWT | 8 | KAPPA |
| I.75′CL | QQGFSDRS | 8 | KAPPA |
| II.14′CL | MQATQFVT | 8 | KAPPA |
| III.7′CL | QRCKGMFS | 8 | KAPPA |
| SPA3-16′CL | QQYGGSPW | 8 | KAPPA |
| VL CLONE 52′CL | CRSHWPYT | 8 | KAPPA |
| 6F5-01′CL | QQYYSTPP | 8 | KAPPA |
| 6F7-42′CL | QQCNTNPP | 8 | KAPPA |
| 6F8-01′CL | QQYYSTPP | 8 | KAPPA |
| 6F9-31′CL | QQYYSVPP | 8 | KAPPA |
| D7′CL | QQYDSLVT | 8 | IGG1-KAPPA |
| HuVK′CL | HQYLSSWT | 8 | KAPPA |
| BC-26′CL | MQGIHLLT | 8 | KAPPA |
| VkLaE34′CL | QHYYGTPH | 8 | KAPPA |
| FL9-K | QQYNTYPT | 8 | KAPPA |
| HSC7 | QEFGDSGT | 8 | IGG |
| H8C28 | QQYGGSPW | 8 | IGG |
| SEGcPB | QQYGSSRT | 8 | IGM-KAPPA |
| P3′CL | QQYDSLPT | 8 | KAPPA |
| A5K3′CL | QQYGSVFT | 8 | IGM |
| BZ1K1′CL | QQYNSYCS | 8 | IGM |
| BZ2K1′CL | QQYYSTPL | 8 | IGM |
| D11K3′CL | QQYNDWPT | 8 | IGM |
| D17K2′CL | MQNIQFPT | 8 | IGM |
| F21K1′CL | QQYDNLPP | 8 | IGM |
| F22K3′CL | QLLR?LRT | 8 | IGM |
| SCFV198′CL | YQYNNGYT | 8 | KAPPA |
| KC25L′CL | QQRSNWPT | 8 | KAPPA |
| ASSYN13′CL | QQYGTSHT | 8 | KAPPA |
| BCPBL1′CL | QQYNHWPS | 8 | KAPPA |
| BCPBL4′CL | QQYGSLYT | 8 | KAPPA |
| BCPBL6′CL | QQNKDWPL | 8 | KAPPA |
| BCSYN6′CL | QQFGTSLT | 8 | KAPPA |
| ITPBL14′CL | QQRSNWWT | 8 | KAPPA |
| ITPBL2′CL | QQCSNWPT | 8 | KAPPA |
| SP10′CL | QQYGSSPT | 8 | KAPPA |

TABLE 3

| # Name | L3 | Length-L3 | Class |
|---|---|---|---|
| 8E10′CL | QQYGSSPSIT | 10 | IGM-KAPPA |
| III-2R′CL | QKYNSAPPST | 10 | IGM-KAPPA |
| II-1′CL | QEYNNWPLWT | 10 | KAPPA |
| 35G6′CL | QQYGGSPPWT | 10 | IGM-KAPPA |
| GF4/1.1′CL | HEYNGWPPWT | 10 | IGG3-KAPPA |
| RF-TS5′CL | QQYNSYSPLT | 10 | IGM-KAPPA |
| O-81′CL | MQHTHWSPIT | 10 | IGM-KAPPA |
| mAb114′CL | QHYNNWPPWT | 10 | IGM-KAPPA |
| HIV-B8′CL | QQSYNTPPWT | 10 | IGG1-KAPPA |
| HIV-b8′CL | QQSYNTPPWT | 10 | IGG1-KAPPA |
| TT117′CL | QHYGSSPPWT | 10 | IGG1-KAPPA |
| HIV- | QQHNNWPPLT | 10 | IGG1-KAPPA |
| loop13′CL | | | |
| HIV-s3′CL | QVYGQSPVFT | 10 | IGG1-KAPPA |
| 1-185-37′CL | QQYGSSPMYT | 10 | IGM-KAPPA |
| 1-187-29′CL | QQYGSSPMYT | 10 | IGM-KAPPA |
| HIV-s5′CL | QRFGTSPLYT | 10 | IGG1-KAPPA |
| HIV-b3′CL | QQYGDSPLYS | 10 | IGG1-KAPPA |
| GER | QQYDDWPPIT | 10 | IGG-KAPPA |
| BLI′CL | QQLNSYPPYT | 10 | IGM-KAPPA |
| 2A4′CL | QQSYSTPPDT | 10 | IGG |
| 0-16′CL | QHYNNWPPSS | 10 | KAPPA |
| mAb48′CL | QHYNRLPPWT | 10 | IGG3-KAPPA |
| 447.8H′CL | QQYDRSVPLT | 10 | KAPPA |
| GP13′CL | QQYYTTPTYT | 10 | IGG1-KAPPA |
| M37GO37′CL | QQYYTTPPLT | 10 | IGG-KAPPA |
| 9500′CL | QQLYSYPHLT | 10 | IGM-KAPPA |
| 9702′CL | CQQYGSSRWT | 10 | IGG-KAPPA |
| GSD2B5B10′CL | MQALQTPMST | 10 | KAPPA |
| MD2F4′CL | QQRSEWPPLT | 10 | KAPPA |
| GAN4B.5′CL | QQYDTSPAWT | 10 | KAPPA |
| NANUC-2′CL | QQYGSSQGFT | 10 | IgG1-kappa |
| SOL10′CL | MQSIQLPRWT | 10 | KAPPA |
| AB1/2′CL | QHYGLSPPIT | 10 | IGG1-KAPPA |
| AB4′CL | QEYGSSPPRT | 10 | IGG1-KAPPA |
| RH-14′CL | SSYRSSSTRV | 10 | IGG1-LAMBDA |
| AB1/2′CL | QHYGLSPPIT | 10 | IGG1-KAPPA |
| Ab4′CL | QEYGSSPPRT | 10 | IGG1-KAPPA |
| L55-81′CL | QQYYTTLPLT | 10 | IGM-KAPPA |
| B3 | SSYSSTTRVV | 10 | IGG |
| HUL-mRF′CL | QQYGSSPQTF | 10 | IGM-KAPPA |
| 25C1′CL | FCQYNRYPYT | 10 | KAPPA |
| LC4aPB | LQRSNWGEVT | 10 | IGM-KAPPA |
| LC4bPB | QQRSNWGEVT | 10 | IGM-KAPPA |
| LC4cPB | QQRSNWGEVT | 10 | IGM-KAPPA |
| mAb3.B6′CL | QQYGSSPLFT | 10 | IGM |
| mAb1.C8′CL | CSYTSSSTLV | 10 | IGM |
| P9′CL | QQRSNWPPIT | 10 | KAPPA |
| 21H9′CL | QQSYNTLSLT | 10 | IGG1-KAPPA |
| 19A5′CL | QHYGNSPPYT | 10 | IGG1-KAPPA |
| 43F10′CL | QQSHKTLAWT | 10 | IGG1-KAPPA |
| FON′CL | MQGTYWPPYT | 10 | IGM-KAPPA |
| Hu PR1A3 | HQYYTYPLFT | 10 | KAPPA |
| hu PR1A3 | HQYYTYPLFT | 10 | KAPPA |
| CLL-412′CL | QQSYSTPPWT | 10 | IGG-KAPPA |
| MEV | QQSYTNPEVT | 10 | KAPPA |
| SON | QQYGSSPPYT | 10 | IGM-KAPPA |
| HEW′′CL | QQYGSSPRYT | 10 | KAPPA |
| HEW′CL | QQYGSSPRYT | 10 | KAPPA |
| JH′ | QQFGNSPPL? | 10 | IGG2-KAPPA |
| HG2B10K′CL | QQYAGSPPVT | 10 | IGG-KAPPA |
| CLL′CL | QQYNNWPPWT | 10 | IGM-KAPPA |
| slkv12′CL | QQYNNWPPWT | 10 | KAPPA |
| bkv6′CL | QQRSNCSGLT | 10 | KAPPA |
| slkv11′CL | QQYNNWPPWT | 10 | KAPPA |
| slkv13′CL | QQYNNWPPWT | 10 | KAPPA |
| bkv7′CL | QQYNNWPPCT | 10 | KAPPA |
| bkv22′CL | QQYNNWPPWT | 10 | KAPPA |
| bkv35′CL | QQRSFWPPLT | 10 | KAPPA |
| MD3.3′CL | QQRSNWPSIT | 10 | KAPPA |
| MD3.1′CL | QQRSNWPPLT | 10 | KAPPA |
| GA3.6′CL | QQRTNWPIFT | 10 | KAPPA |
| M3.5N′CL | QQRSNWPPGT | 10 | KAPPA |
| MD3.4′CL | QQYNNWPPLT | 10 | KAPPA |
| M3.1N′CL | QQYNNWPTWT | 10 | KAPPA |
| GA3.4′CL | QQRMRWPPLT | 10 | KAPPA |
| MD3.7′CL | QQYGSSPKWT | 10 | KAPPA |
| MD3.9′CL | QQYGSSPQYT | 10 | KAPPA |
| GA3.1′CL | QQYGSSPPYT | 10 | KAPPA |
| bkv32′CL | QQYDRSLPRT | 10 | KAPPA |
| GA3.5′CL | QQYGNSPLFS | 10 | KAPPA |
| GA3.8′CL | QQYGGSPLFS | 10 | KAPPA |
| E29.1 | QQYNNWPTWT | 10 | IGM-KAPPA |
| KAPPA′CL | | | |
| R5A3K′CL | MQALQTLGLT | 10 | IGM-KAPPA |
| R1C8K′CL | MQALQTLGLT | 10 | IGG-KAPPA |
| I.24′CL | QQSHSAPPYT | 10 | KAPPA |
| III.12′CL | QQYGSSPLFT | 10 | KAPPA |
| III.5′CL | QQYNDWPPWT | 10 | KAPPA |
| I.18′CL | QQYNGNSPLT | 10 | KAPPA |
| I.67′CL | QQLNTYPPWT | 10 | KAPPA |
| III.6′CL | HKYGGSPPYT | 10 | KAPPA |
| II.65′CL | MQDTHWPPWT | 10 | KAPPA |
| III.14′CL | QHYGRSPPLT | 10 | KAPPA |
| 424.F6.24′CL | QQYGNSPPYT | 10 | KAPPA |
| T33-5′CL | QQYGSSPPYT | 10 | IGM-KAPPA |
| AL-MH | QQYFNVPPVT | 10 | KAPPA |
| AL-Es305 | QHYHNLPPTT | 10 | KAPPA |
| L47′CL | IQGTHWPQYT | 10 | IGM-KAPPA AND |
| | | | LAMBDA |
| F29′CL | QQYGSSRALT | 10 | IGM-KAPPA AND |
| | | | LAMBDA |
| G28′CL | QQYYSTPSYT | 10 | IGM-KAPPA AND |
| | | | LAMBDA |
| G21′CL | MQALQTLMCS | 10 | IGM-KAPPA AND |
| | | | LAMBDA |
| VL CLONE | QQSYSTPPLT | 10 | KAPPA |
| 45′CL | | | |
| VL CLONE | QQSYSTPPIT | 10 | KAPPA |
| 48′CL | | | |
| VL CLONE | QQYGGSLPIT | 10 | KAPPA |
| 56′CL | | | |
| C9′CL | QQYGSSTPLT | 10 | IGG1-KAPPA |
| ITC88′CL | QQRSSWPPLT | 10 | KAPPA |
| AC18′CL | QQRYSWPPLT | 10 | KAPPA |
| AC31′CL | QQRYNWPPLT | 10 | KAPPA |
| AC32′CL | QQRSNWPPLT | 10 | KAPPA |
| AC37′CL | QQRSSWPPLT | 10 | KAPPA |
| B′20 | QQYNNWPPWT | 10 | IgM-VkIIIa |
| (Humkv328- | | | |
| Jk1)′CL | | | |
| B9601 (Vg- | QQRSNWPPYT | 10 | IgM-VkIIIa |
| Jk2)′CL | | | |
| MF8 | QQYNNWPPWT | 10 | IgM-VkIIIa |
| (Humkv328- | | | |
| Jk1)′CL | | | |
| B′2 | QQYNNWPPWT | 10 | IgM-VkIIIa |
| (Humkv328- | | | |
| Jk1)′CL | | | |
| kappa1′CL | QQYGSSPPIT | 10 | IGG2-KAPPA |
| kappa2′CL | QQYNNWPPIT | 10 | IGG2-KAPPA |
| kappa3′CL | QQRSSWPPIT | 10 | IGG2-KAPPA |
| kappa4′CL | QQYGSSPRVT | 10 | IGG2-KAPPA |
| kappa5′CL | QQYNTNSPIS | 10 | IGG2-KAPPA |
| kappa7′CL | QNYGSSPRIT | 10 | IGG2-KAPPA |
| kappa8′CL | QQYGSSPPIT | 10 | IGG2-KAPPA |
| ToP218′CL | MQSIQLPRFT | 10 | KAPPA |
| ToP241′CL | MQSVQLPRFT | 10 | KAPPA |
| ToP309′CL | MQSVQLPRFT | 10 | KAPPA |
| L1236K3′CL | QQYDKWPPVT | 10 | KAPPA |
| SOL1′CL | MQSIQFPRWT | 10 | KAPPA |
| BC-2′CL | MQGIHLPPYI | 10 | KAPPA |
| P3′CL | NQGTQWLLYT | 10 | KAPPA |
| P5′CL | QQYNSYAPYT | 10 | KAPPA |
| AB1/2′CL | QHYGLSPPIT | 10 | IGG-KAPPA |
| AB4′CL | QEYGSSPPRT | 10 | IGG-KAPPA |
| MH | QQYFNVPPVT | 10 | KAPPA |
| FL6-K | QQLTSYPPWT | 10 | KAPPA |
| FL2-K | QQVNSYPGLT | 10 | KAPPA |
| FL4-K | QQVFSYPGIT | 10 | KAPPA |
| FL1-K | QQYTSLPGIT | 10 | KAPPA |
| MM4-K | QHSYSTLPLT | 10 | KAPPA |
| MM9-K | QQYYNIPYIT | 10 | KAPPA |
| HSC4 | QLYGSSPRVT | 10 | IGG |
| HSC11 | QQYANWPPIT | 10 | IGG |
| HSC13 | QQYNISPRDT | 10 | IGG |
| HSC23 | QQFGSSPLIT | 10 | IGG |
| HSC35 | QQYGDFPRVT | 10 | IGG |
| REV | QQYGDWPPYT | 10 | KAPPA |
| BLU | QQYYTTLSWT | 10 | KAPPA |
| BK2′CL | QQYNKWPPLT | 10 | KAPPA |
| GK6′CL | MQGTHWLPVT | 10 | IGG-KAPPA |
| L1236K3′CL | QQYDKWPPVT | 10 | KAPPA |
| P1′CL | QQYDNLPPIH | 10 | KAPPA |
| H01′CL | QQLNNYPPFT | 10 | KAPPA |
| I01′CL | QQSYSTPPYT | 10 | KAPPA |
| I10′CL | QQSYSTPPYS | 10 | KAPPA |
| I12′CL | QQSYSTPPYT | 10 | KAPPA |
| 126TP14K′CL | QQYNNWLPFT | 10 | IGG-KAPPA |
| L32′CL | AAWDDSLTLM | 10 | IGM-KAPPA |

For insertion of CDR3s, single oligonucleotides encoding each of the CDR3s of table H from the plus strand were synthesised with 12 homologous nucleotides added to each termini for annealing to the consensus VH and VL genes. In addition to these CDR sequences, CDRs from the antibody E25 (see example 4) were included. These primers were extended and secondary primers were added to introduce directly adjacent to the N and C termini of the VH and VL genes (without C regions) 5′NotI-3′XbaI sites for VH and 5′SpeI-3′BamHI for VL. Prior to cloning, a further pair of complimentary primers was used to insert the linker sequence (G1y4Ser)3 between VH and VL whilst maintaining XbaI and SpeI sites. Full-sized VH-linker-VL fragments were digested with NotI and BamHI and were cloned into NotI-BamHI digested pBluescript II KS(+/−) (Stratagene, Amsterdam, Netherlands).

Individual Bluescript clones were picked, plasmid DNA was purified and dispensed robotically into 96 well plates as described in WO99/11777. DNAs were then subjected to IVTT including tRNA-biotinyl-lysine and further robotically arrayed onto a streptavidin surface as described in WO99/11777. The immobilised initial scFv library of 10,000 independant clones was then screened by incubation with recombinant human IgE Fab (see example 4). Wells were blocked with PBS/3% BSA at room temperature for 1 hour, washed three times in PBS and treated with 5 ug/ml human IgE Fab in PBS/3% BSA for 1 hour. Wells were then washed a further three times in PBS and treated with 5 ug/ml alkaline phosphatase-labelled chimeric anti-IgE (example 4) in PBS/3% BSA for 1.5 hrs. Wells were further washed five times in PBS and colour developed using the substrates 5-bromo-1-chloro-3-indolyl phosphate and nitro blue tetrazolium (Roche Molecular) for visualization. A strong signal observed at a frequency of 1 of 9600 wells was shown to derive from a VH and VL pair both containing E25 CDR3's.

### EXAMPLE 6

### Construction of Composite Mouse Anti-TNFα Antibody

A mouse variable region sequence library was created as described in example 1 for the human library using NCBI Igblast, Kabat and Genbank databases. The reference antibody variable region sequences used was a chimeric anti-TNFα antibody known as Remicade® (Le et al., U.S. Pat. No. 6,277,969) using the variable regions of the mouse cA2 antibody. Segments from the in silico mouse variable region sequence library were selected partly corresponding amino acids in the Remicade® variable region but including variations designed to avoid human T cell epitopes in the sequence in the form of non-self human MHC class II binders measured as in example 1. Composite mouse VH and VL sequences compared to sequences used in the chimeric antibody are shown in FIG. 8 indicating differences of 9 and 16 amino acids in VH and VL respectively between the two antibodies as a result of segment selection for epitope avoidance in the Composite Mouse antibody.

The Composite Mouse and chimeric anti-TNFα antibodies were generated as described in example 1. Comparison of purified antibodies for binding to immobilised human TNFα in a standard ELISA (described in WO 03/042247A2) showed that the Composite Mouse antibody retained the full binding capacity of the chimeric anti-TNFα, antibody (FIG. 9). The immunogenicity of these antibodies was then compared as described in example 2 using 24 HLA-DR typed human blood samples for T cell assays. The results showed that the chimaeric anti-TNFα antibody induced significant proliferative responses (SI greater than 2) in nine of the twenty four healthy donors tested (37.5%) compared to the Composite Mouse anti-TNFα antibody where none of the twenty four donors (0%) induced SI>2. These results indicated that a Composite Mouse antibody comprising segments of variable region sequence derived totally from mouse V regions with selection of such segments to avoid human T cell epitopes could remove the immunogenicity in human T cell assays displayed by the corresponding chimeric antibody without any epitope avoidance measures.

### EXAMPLE 7

### Construction of a Composite Human Anti-TNFα Antibody

The reference mouse variable region heavy and light chain sequences of antibody A2 directed against human TNFα was obtained from U.S. Pat. No. 5,656,272 (FIG. 10. SEQ. IDs No. 1 and No. 2 respectively). A structural model was made of the mouse reference variable regions and amino-acids critical for CDR conformation were identified based upon their distance from the CDRs (<3 Å) and their likely packing close to CDRs. Important, but less critical residues were identified based upon their distance from the CDRs (>3 Å<6 Å) and their likely influence on more critical residues packing closer to the CDRs. A further set of residues were identified based upon their frequency of occurrence in mouse antibody sequences i.e. amino-acids found at a particular location with a frequency of less than 1%.

Human V region sequence segments that included as many of these residues as possible were selected (table 4) to create full-length VH and VL sequences. Alterations were made to these sequences to include all the identified structurally important residues to create sequences to serve as a template for epitope avoidance and Composite Human Antibody design. A preferred sequence for each composite VH and VL was designed to include important residues from the reference mouse antibody. These variable heavy and light chain amino acid sequences are shown in FIGS. 11 and 12. SEQ IDs No. 3 and No. 4 respectively.

TABLE 4
Human Antibody Database Derivation of Sequence
Segments For Primary CHAB Variants
Genbank

| Accession No. | Sequence segment |
|---|---|
| (a) Heavy Chain | |
| CAA61442 | EVQLVESGGGLVQPGCSLKLSC |
| CAD88676 | LSCVASGFIFS |
| CAB37182 | FSNHWM |
| AAS86088 | HWMNWVRQAPGKGLEWVA |
| CAC43592 | AEI |
| ABB54411 | IRSKS |
| AAL96548 | SIN |
| AAK51359 | NSA |
| CAA67405 | SAT |
| CAB87447 | ATHYA |
| AAD30769 | HYAESVKGRFTISRD |
| CAC15703 | RFTISRDDSKSI |
| AAQ05509 | IVYLQM |
| AAT96742 | YLQMTDLR |
| AAD20526 | LRTEDTGVYYC |
| CAB44788 | VYYCSRNY |
| AAO38724 | NYYGS |
| AAK14004 | GSTY |
| AAD20470 | TYDYWGQGT |
| AAB32435 | DYWGQGTTVTVSS |
| (b) Light Chain | |
| CAC06686 | DILLTQ |
| AAX57564 | LTQSPAILSLSPGERATLSC |
| X72820 | LSLSPGERATLSCRASQ |
| AAC15439 | QFV |
| AAZ09058 | VGSS |
| Z84907 | SSI |
| AAL10835 | IHWYQQK |
| AAQ21835 | QQKPNQSPKLLIK |
| M27751 | LLIKYAS |
| AAY16612 | YASE |
| AAR89591 | ES |
| AAD19534 | SM |
| AAV71416 | MSG |
| AAZ09098 | GIP |
| CAG27043 | PSRFSGSGSGTDFTLTINSLE |
| AAQ21937 | SLESEDAA |
| AAC41988 | ADYYCQQ |
| AAY33370 | YYCQQSHS |
| AAD19457 | HSWP |
| AAQ55271 | WPFTFGQGT |
| AAW69118 | TFGQGTNLEIK |

The composite heavy and light chain variable region sequences were scanned for the presence of potential T cell epitopes using a variety of in silico methods (e.g. Propred [http://imtech.res.in/raghava/propred/index.html], Peptide Threading [www.csd.abdn.ac.uk/˜gjlk/MHC-thread], SYFPEITHI (www.syfpeithi.de), MHCpred (www.jenner.ac.uk/MHCPred/) and compared to homologous human germ-line framework region sequences in conjuction with reference mouse CDRs.

The following heavy chain variable region variants were made (see FIG. 11):

SEQ. ID. No. 5 contains the following changes with respect to SEQ. ID. No. 3: T82aN+R83K.

SEQ. ID. No. 6 contains the following changes with respect to SEQ. ID. No. 3: T82aN+R83K+D82bS

SEQ. ID. No. 7 contains the following changes with respect to SEQ. ID. No. 3: T82aN+R83K+D82bS+V23A.

SEQ. ID. No. 8 contains the following changes with respect to SEQ. ID. No. 3: T82aN+R83K+D82bS+V23A+V78A

The following light chain variable region variants were made (see FIG. 12):

SEQ. ID. No. 9 contains the following changes with respect to SEQ. ID. No. 4: I10T+N103R.

SEQ. ID. No. 10 contains the following changes with respect to SEQ. ID. No. 4: I10T+N103R+S80A.

SEQ. ID. No. 11 contains the following changes with respect to SEQ. ID. No. 4: I10T+N103R+S80A+N41D.

For construction of a control chimeric antibody, the nucleotide sequences that translate to give SEQ. IDs No. 1 and No. 2 were constructed using a series of overlapping 40 mer synthetic oligonucleotides. The V region sequences were flanked by additional 5′ and 3′ sequences to facilitate cloning into mammalian expression vectors. The sequences of the oligonucleotides are shown in FIG. 13 and FIG. 14

Oligonucleotides were purchased from Sigma-Genosys (Poole, UK) and resuspended at a concentration of 100 μM. 1 μl of each of the heavy chain sense strand oligonucleotides, except the most 5′ oligonucleotide, were mixed together and 1.50 (approx. 1 μg) of the mix was treated with Polynucleotide Kinase (PNK, Invitrogen, Paisley UK) in a 20 μl reaction containing additionally: 2 μl 10× PNK buffer, 2 μl 10 mM ATP, 14 μl H₂O, 0.5 μl (5 units) PNK. The reaction was incubated at 37° C. for 30 min and the enzyme inactivated by heating at 70° C. for 20 min. The heavy chain antisense, light chain sense and antisense oligonucleotides were similarly phosphorylated. The 5′ oligonucleotide from each set was diluted 1 in 9 with H₂O and 1.5 μl added to the appropriate reaction mix. Each reaction was then diluted to 0.5 ml and spin dialyzed in an Amicon microcon YM3 concentrator for 90 min at 8000 rpm until the volume was not more than 44 μl.

The sense and antisense mixes for the heavy chain, and those for the light chain, were combined and made up to 88 μl with H₂O. 10 μl 10× Ligase Chain Reaction (LCR) buffer and 2 μl Pfu ligase (8 units, Stratagene, Cambridge UK) were added to each reaction and incubated as follows in a programmable heating block: 94° C. for 4 min, then 60° C. for 3 min for 1 cycle followed by 20 cycles of 94° C. for 39 sec. then 60° C. for 2 min. Finally the reactions were incubated for 5 min at 60° C. 10 μl of each LCR was run through a 1% agarose gel stained with ethidium bromide and compared to 1 Kb ladder markers (Invitrogen). A smear of ligated DNA was observed in each lane, surrounding a faint specific band of approximately 400 bp.

The heavy and light chain LCRs were amplified via PCR using as primers SEQ. ID. No.s 12 and 22 for the heavy chain and SEQ. ID. No.s 33 and 43 for the light chain. The following were included in each reaction: 5 μl LCR, 5 μl 10× Expand HiFi buffer (Roche, Lewes UK), 1 μl 10 mM NTP mix, 0.25 μl each primer (from 100 μM stocks), 0.5 μl Expand HiFi polymerase (3 units, Roche) and 38 μl H₂O. The reactions were cycled as follows: 94° C. 2 min followed by 20 cycles of 94° C. for 30 sec, 60° C. for 30 sec and 72° C. for 30 sec. Finally the reaction was incubated for 5 min at 72° C. The yield and specificity of the reaction was confirmed by agarose gel electrophoresis, as above. Specific, sharp bands at approximately 400 bp were observed for each reaction.

The reaction products were purified using a Qiagen PCR purification kit and each eluted in 30 μl H₂O. The heavy chain product was digested in a standard reaction with restriction enzymes Mlu 1 and Hind III and the light chain product was digested with BssH II and BamH I. The reaction products were again purified using a Qiagen PCR purification kit and each eluted in 30 μl H₂O.

The light chain expression vector pANT08 was based upon a pAT153 backbone and contains in the following order: CMV immediate/early enhancer promoter −590 to +7, a 30 nt 5′ UTR derived from a highly expressed mouse antibody light chain RNA, a mouse consensus light chain signal sequence incorporating a BssH II restriction site near the variable region start codon, a short linker (in place of a variable region) to a human composite intron containing 33 nt from the variable region splice site to a BainH I restriction site followed by a fragment of human genomic DNA containing 343 nt of the intron preceding the human constant Kappa (CK) region gene, the CK gene and CK polyA.

The heavy chain expression vector pANT09 was similar to pANT08 through the promoter region, which is followed by: a 62 nt 5′ UTR derived from the heavy chain counterpart of that described above, a mouse heavy chain consensus signal sequence that incorporates a Mlu I restriction site near the variable region start codon, a short linker (in place of a variable region) to the variable region splice site immediately followed by a fragment of human genomic DNA from a Hind III restriction site located in the intron 211 nt upstream of the CH1 gene, to the end of the CH region poly A site. This fragment includes the CH1, hinge, CH2 and CH3 introns and exons of human IgG1. This vector also included a gene for dihydrofolate reductase, controlled by an SV40 promoter and polyA signal, for resistance to methotrexate.

2 μg each vector was digested with the relevant restriction enzymes in standard reactions in a total volume of 30 μl. Each reaction was run through a 1% agarose gel, as above, and the vector specific bands (6.0 Kbp heavy chain and 4.2 Kbp light chain) were excised from the gel and purified using a Qiagen gel extract kit and eluted in 30 μl H₂O.

1 μl each digested vector was ligated to 3 μl of the corresponding digested variable gene PCR product using a Ligafast kit (Promega, Southampton UK). 2.5 μl each ligation reaction was transformed into sub-cloning efficiency competent XL1-blue (Stratagene), as instructed by the manufacturer, and plated onto LB agar plates containing 100 μg/ml ampicillin and incubated overnight at 37° C. Ten bacterial colonies from each ligation were inoculated into 10 ml 2× YT broth containing 100 μg/ml ampicillin and grown overnight at 37° C. with shaking. Plasmid was purified from 1.5 ml each overnight culture using a Qiagen plasmid preparation kit and each eluted in 50 μl H₂O. The plasmids were sent to a contract sequencing facility and sequenced with a standard CMV promoter primer and clones with the correct V region gene sequence identified.

For construction of Compsoite Human Antibodies, the nucleotide sequences that translate to give SEQ. IDs No. 3 and No. 4 were constructed using a series of overlapping 40 mer synthetic oligonucleotides. The sequences of the oligonucleotides are shown in FIG. 15 and FIG. 16. The nucleotide sequence that translates to give SEQ. ID. No. 5 was constructed via overlap PCR using oligonucleotide primers SEQ. ID. No.s 94 and 95 (FIG. 17) together with oligonucleotides SEQ. ID. No.s 53 and 63 and the plasmid DNA of the primary Composite Human Antibody heavy chain variant as template. Two PCR reactions were done using as primer pairs either SEQ. ID. No.s 53 and 95, or SEQ. ID. No.s 94 and 63. The following were included in each reaction: 1 μl (100 ng) plasmid template, 5 μl 10× Expand HiFi buffer (Roche), 1 μl 10 mM NTP mix, 0.25 μl each primer (from 100 μM stocks), 0.5 μl Expand HiFi polymerase (3 units, Roche) and 42 μl H₂O. The reactions were cycled as follows: 94° C. 2 min followed by 20 cycles of 94° C. for 30 sec, 60° C. for 30 sec and 72° C. for 30 sec. Finally the reaction was incubated for 5 min at 72° C. The entire reactions were electrophoresed through a 1% agarose gel and the specific bands of 295 bp and 126 bp were excised and purified using a Qiagen gel extraction kit. The DNAs were eluted in 30 μl H₂O.

The two purified fragments were joined in a PCR reaction using oligonucleotide primers SEQ. ID. No.s 53 and 63 using PCR conditions as described above, except that the template used was 1μl 295 bp product and 1 μl 126 bp product, hence the amount of H₂O was reduced to 41 μl. The joined PCR product of 396 bp was purified using a Qiagen PCR purification kit and was eluted in 30 μl H₂O.

The nucleotide sequence that translates to give SEQ. ID. No. 6 was constructed via overlap PCR using oligonucleotide primers SEQ. ID. No.s 96 and 97 (FIG. 17) together with oligonucleotides SEQ. ID. No.s 53 and 63 and the plasmid DNA of the primary Composite Human Antibody heavy chain variant as template. Two PCR reactions were done using as primer pairs either SEQ. ID. No.s 53 and 97, or SEQ. ID. No.s 96 and 63. The first stage PCRs were done as described above and yielded fragments of 295 bp and 126 bp. These fragments were purified, joined together and repurified, also as described above.

The nucleotide sequence that translates to give SEQ. ID. No. 7 was constructed via overlap PCR using oligonucleotide primers SEQ. ID. No.s 98 and 99 (FIG. 17) together with oligonucleotides SEQ. ID. No.s 53 and 63 and the PCR product for SEQ. ID. No. 6 as template. Two PCR reactions were done using as primer pairs either SEQ. ID. No.s 53 and 99, or SEQ. ID. No.s 98 and 63. The first stage PCRs were done as described above and yielded fragments of 98 bp and 318 bp. These fragments were purified, joined together and repurified, also as described above.

The nucleotide sequence that translates to give SEQ. ID. No. 8 was constructed via overlap PCR using oligonucleotide primers SEQ. ID. No.s 100 and 101 (FIG. 17) together with oligonucleotides SEQ. ID. No.s 53 and 63 and the PCR product for SEQ. ID. No. 7 as template. Two PCR reactions were done using as primer pairs either SEQ. ID. No.s 53 and 101, or SEQ. ID. No.s 100 and 63. The first stage PCRs were done as described above and yielded fragments of 270 bp and 155 bp. These fragments were purified, joined together and repurified, also as described above.

Each of the above PCR products was digested with Mlu I and Hind III and ligated into similarly digested pANT09. The ligations were transformed and plated, colonies picked, plasmids prepared and sequenced all as described above.

The nucleotide sequence that translates to give SEQ. ID. No. 9 was constructed via PCR using oligonucleotide primers SEQ. ID. No.s 102 and 103 (FIG. 17) and the plasmid DNA of the primary Composite Human Antibody light chain variant as template. A single PCR reaction was done, as described for the heavy chain variants, that yielded a product of 383 bp. The entire reaction was electrophoresed through a 1% agarose gel and the specific band was excised and purified using a Qiagen gel extraction kit. The DNA was eluted in 30 μl H₂O.

The nucleotide sequence that translates to give SEQ. ID. No. 10 was constructed via overlap PCR using oligonucleotide primers SEQ. ID. No.s 104 and 105 (FIG. 17) together with oligonucleotides SEQ. ID. No.s 74 and 84 and the PCR product for SEQ. ID. No. 9 as template. Two PCR reactions were done using as primer pairs either SEQ. ID. No.s 74 and 105, or SEQ. ID. No.s 104 and 84. The first stage PCRs were done as described above for the heavy chain variants and yielded fragments of 265 bp and 139 bp. These fragments were purified, joined together to create a product of 383 bp and repurified, also as described above for the heavy chain variants.

The nucleotide sequence that translates to give SEQ. ID. No. 11 was constructed via overlap PCR using oligonucleotide primers SEQ. ID. No.s 106 and 107 (FIG. 17) together with oligonucleotides SEQ. ID. No.s 74 and 84 and the PCR product for SEQ. ID. No. 10 as template. Two PCR reactions were done using as primer pairs either SEQ. ID. No.s 74 and 107, or SEQ. ID. No.s 106 and 84. The first stage PCRs were done as described above for the heavy chain variants and yielded fragments of 148 bp and 256 bp. These fragments were Purified, joined together to create a product of 383 bp and repurified, also as described above for the heavy chain variants.

Each of the above PCR products was digested with BssH II and BamH I and ligated into similarly digested pANT08. The ligations were transformed and plated, colonies picked, plasmids prepared and sequenced all as described above.

CHO-K1 cells (ATCC #CCL-61) were propagated in high glucose DMEM containing 10% FCS, L-glutamine, sodium pyruvate and L-proline. Near confluent cultures were harvested for transfection using Lipofectamine 2000 as instructed by the manufacturer (Invitrogen). Transfections were done in 48 well plates seeded with 200 μl cells at 3×10⁵ cells/ml using a total of 0.5 μg plasmid DNA comprising 0.3 μg heavy chain construct and 0.2 μg light chain construct.

Transfections were incubated at 37° C./5% CO₂ for 48 to 72 h before harvesting the supernatants. Antibody expression was quantified by ELISA using: a mouse monoclonal anti-human IgG capture antibody, human IgG1/Kappa standards and HRP conjugated goat anti-human Kappa light chains as detection antibody (all reagents from Sigma).

All combinations of heavy and light chains were transfected (i.e. 6 heavy chains×5 light chains=30 transfections). Antibody expression levels were generally in the range of 0.5 to 2.0 μg/ml, however no expression was observed with heavy chain SEQ. ID. No. 8.

The expressed antibodies were tested for their ability to neutralize the activity of human TNFα using TNF-sensitive WEHI-164 cells (Espevik et al., J. Immunol. Methods 1986, 95, 99-105). Cells were plated in 1 μg/ml actinomycin D at 5×10⁴ cells per well in 96-well microtiter plates for 3-4 hours. Cells were exposed to 40 pM human TNFα and varying concentrations of the chimeric antibody (range 1 ng/ml to 500 ng/ml) to create a standard curve. The various combinations of heavy and light chains were tested at a single concentration point of 25 ng/ml that had previously been determined as the ED₅₀ of the chimeric antibody. All assays were done in triplicate.

The mixtures were incubated overnight at 37° C. Cell viability was determined by adding 3-[4,5-dimethyl-thiazol-2-yl]-2, 5-diphenyltetrazoliumbromide dye (MTT) to a final concentration of 0.5 mg/ml, incubating for 4 hours at 37° C., lysing the cells in 0.1M HCl, 0.1% SDS and measuring the optical density at 550 nm wavelength.

The optical densities from the heavy and light chain combinations were used to calculate the apparent antibody concentrations from the standard curve. The apparent concentration of the chimeric was divided by that of each of the variant combinations to give a fold difference value. Values lower than that for the chimeric indicated that those combinations were more effective at protecting the cells from TNFα cytotoxicity, whereas higher values indicated that they were less effective. The values for all combinations are shown in Table 5.

TABLE 5
Ratio of Activities of Composite Human Antibody
Variants compared to Chimeric Antibody

| SEQ. ID. No. | 1 | 3 | 5 | 6 | 7 | 8 |
|---|---|---|---|---|---|---|
| 2 | 1.00 | 1.38 | 1.24 | 1.20 | 1.02 | ND |
| 4 | 1.51 | 2.28 | 1.28 | 1.38 | 1.05 | ND |
| 9 | 1.28 | 2.14 | 1.32 | 1.77 | 0.95 | ND |
| 10 | 1.31 | 2.51 | 1.17 | 1.63 | 0.98 | ND |
| 11 | 16.90 | 15.15 | 196.49 | 134.08 | 105.61 | ND |

The following Composite Human Antibody heavy and light chain combinations gave fold differences close to 1.0: SEQ. ID. No.s 5/10, SEQ. ID. No.s 7/4, SEQ. ID. No.s 7/9, SEQ. ID. No.s 7/10. These combinations were selected for further study.

The expression plasmids carrying the sequences selected above were transfected into NS0 cells (ECACC No. 85110503). The cells were grown in high glucose DMEM containing L-glutamine, sodium pyruvate, 5% ultra low IgG FCS and pen/strep. Cells were harvested during log phase of growth, spun down and resuspended at 5×10⁶ cells/ml in fresh growth media. 750 μl cells were mixed with a total of 30 μg of each plasmid pair, which had been linearised with Ssp I, in 50 μl H₂O. The cell/plasmid mixture was transferred to a 4 mm gap cuvette and electroporated using an Equibio Easyject Plus at 250V, 1500 μF, infinite resistance. The electroporate was immediately transferred to 25 ml pre-warmed growth media and plated out in 5×96 well flat bottomed plates at 100 μl/well. The plates were incubated at 37° C./5% CO₂. 48 h post-electroporation, 50 μl media containing 300 nM methotrexate was added to each well to give a final concentration of 100 nM. 7 days post-electroporation a further 50 μl of media containing 100 nM methotrexate was added to each well.

Approximately 2 week post-electroporation, the media in some wells began to turn yellow, indicating transfected colony growth. Media from these wells were tested for antibody expression using the anti-human IgG Fc capture/anti-human Ig Kappa light chain HRP conjugate detection ELISA. The test samples were compared to a human IgG1/Kappa standard and antibody expression levels estimated. Colonies expressing useful amounts of antibody were expanded in media containing 200 nM methotrexate.

Antibodies were purified from 500 ml culture media via protein A affinity chromarography followed by size exclusion chromatography using Sephacryl S200. The purified antibodies were quantified by UV absorbance at 280nm, assuming that OD₂₈₀1=1.4 mg/ml.

Purified chimeric and composite antibodies were tested for activity via the WEHI-164 protection assay described in example 4 above. Each antibody was tested over the full concentration range previously used to create the standard curve (see FIG. 18). Composite Human Antibody 7/10 (i.e. containing SEQ. ID. No.s 7 and 10) was found to be the most active variant and had the same activity as the chimeric antibody. Composite Human Antibodies 7/9 and 5/10 had similar activity that was slightly reduced compared to the chimeric, and Composite Human Antibody 7/4 was the least active.

Therefore since Composite Human Antibody 7/10 was predicted to have the most favourable MHC class II binding profile and was the most active variant, this was selected for testing in a time course T cell proliferation assay. Human PBMCs were prepared from buffy coats derived from human blood donations via two rounds of Ficoll density centrifugation. The prepared PBMC were resuspended at a density of 3×10⁷ cells/ml in 1 ml aliquots in 90% human AB serum/10% DMSO, and stored under liquid nitrogen. PBMC were tissue typed using a Dynal Allset® PCR typing kit.

The lead Composite Human Antibody was compared to the chimeric antibody in whole protein T cell assays using human PBMC from 20 healthy donors. PBMC from each donor were thawed, washed and resuspended in AIM V serum free lymphocyte growth media. On day 1, 50 μg protein was added to 2 ml bulk cultures of 4×10⁶ PBMC in 24 well plates, and triplicate 100 μl aliquots were removed and transferred to 96 well plates on days 6 to 9. Each aliquot was pulsed with 75 μl media containing 1 μCi tritiated thymidine for 24 h, before harvesting and measuring incorporation of radioactivity. Results were normalised by calculation of the Stimulation Index (SI). Coverage of a wide range of HLA DR allotypes was achieved by selecting donors according to individual MHC haplotypes.

The results of the time-course assay are shown in FIG. 19 and demonstrated that the chimeric antibody (FIG. 19(a)) elicits a T cell response (SI>=2) on at least one day in 10 of the 20 donors. In contrast, Composite Human Antibody (FIG. 19(b)) failed to elicit a response in any of the donors at any time point. Therefore a non-immunogenic Composite Human Antibody was successfully constructed from segments of human antibodies using a mouse anti-TNFα antibody (A2) as reference.

### EXAMPLE 8

### Construction of a Composite Type I Ribosome Inhibitory Protein

Composite variants of the plant type I Ribosome Inhibitory Protein (RIP) bouganin (derived from Bougainvillea spectabilis) were generated using methods described in WO2005090579. The location of T cell epitopes in bouganin was tested by analysis of overlapping 15 mer peptides as in WO2005090579 and the peptides of SEQ ID 11-13 in table 6 (corresponding to residues 121-135, 130-144 and 148-162) were identified as epitopes. Bouganin was cloned from leaf tissue from a Bougainvillea spectabilis plant. mRNA was extracted using a polyA Tract System 1000 kit (Promega) from 100 mg tissue as instructed by the manufacturer. cDNA was synthesised from the mRNA template using an AccessQuick RT-PCR system (Promega) with the following primers: ATGTACAACACTGTGTCATTTAAC and TTATTTGGAGCTTTTAAACTTAAGGATACC. The first primer additionally contains an ATG start codon and the second primer additionally contains a TAA stop codon. The PCR product was cloned using a T/A cloning system (pGEM T Easy, Promega) and several clones were sequenced to identify a correct clone orientated with the transcription direction of the T7 promoter contained within the vector.

TABLE 6
Immunogenic Peptide Sequences of bouganin and
Replacement Human Sequence Segments

| | |
|---|---|
| SEQ ID No. 11: | ¹²¹AKVDRKDLELGVYKL¹³⁵ |
| | AAKAD-CAD39157 |
| | AKADR-AAH01327 |
| | KADRK-XP_372046 |
| | AAKSDR-AAH47411 |
| | KSDRKD-NP_002678 |
| | AAKTD-BAA23704 |
| | AKTDR-AAD00450 |
| | KTDRK-CAH18368 |
| SEQ ID No. 12: | ¹³⁰LGVYKLEFSIEAIHG¹⁴⁴ |
| | ELGPQ-BAC04852 |
| | LGPQK-NP_056013 |
| | GPQKLE-XP_370607 |
| | ELGGK-AAI00815 |
| | LGGKKL-BAD96533 |
| | GGKKLE-AAK68690 |
| | ELGNS-BAB14022 |
| | LGNSKL-BAD98114 |
| | GNSKLE-CAG46875 |
| | ELGQAKL-AAF42325 |
| | LGQAKLE-AAN63404 |
| | ELGQD-CAH71404 |
| | LGQDK-BAC04773 |
| | QDKLE-NP_004000 |
| SEQ ID No. 13: | ¹⁴⁸NGQEIAKFFLIVIQM¹⁶² |
| | GQEQA-CAI95134 |
| | QEQAK-AAH55427 |
| | EQAKF-NP_079390 |
| | GQERA-AAH10634 |
| | QERAK-NP_003153 |
| | ERAKF-AAH14009 |

A series of variants were made containing the human sequence segments identified as shown in table 6. These were constructed using overlap PCR with a high fidelity polymerase (Expand Hi-Fi, Roche). The 5′ and 3′ primers were as above and the PCR products were cloned into the T/A cloning vector, as above, and correct clones identified that were orientated with the transcription direction of the T7 promoter. Clones were assayed for activity in a coupled transcription and translation reaction that included a control DNA expressing a luciferase gene (Luciferase T7 Control, Promega). Since bouganin is a ribosome inactivating protein, it significantly reduces the levels of translation of the luciferase gene and this reduction is conveniently assayed using a luciferase detection system such as Steady-Glo (Promega). Purified wild type or mutant bouganin plasmids were linearised with Not I and diluted to 10 ng/μl. Luciferase T7 Control DNA was diluted to 125 ng/μl. 1 μl each DNA was mixed with 10 μl TnT mix (Promega), 0.25 μl Methionine and 0.25 μl nuclease free water (supplied in TnT kit). Controls were wt bouganin and Luciferase T7 Control only. Reactions were undertaken in triplicate and incubated for 1 hour at 30° C. 5 μl each reaction was transferred to a black walled 96 well luminometer plate and mixed with 45 μl water and 50 μl Steady-Glo reagent. Luminescence was read in a Wallac Microbeta Trilux luminometer. Activity was expressed as a percentage of the luminescence observed from the Luciferase T7 Control DNA alone.

FIG. 20 illustrates the activity profile of a number of different variants. This shows that the most active variants are: V123T in peptide 41; V132P/Y133Q in peptide 44; I152Q in peptide 50. A combined mutant was made containing these 4 mutations and re-tested in the activity assay. The activity of this mutant is indicated by COMB in FIG. 20 and retains approximately 75% of the activity of the wt protein.

Peptides containing the human sequence segments within the active COMB variant corresponding to residues 121-135, 130-144 and 148-162 were synthesised and compared to the corresponding wild type peptides in a time-course T cell assay with human PBMCs from 20 healthy donors as described in example 7. The results showed that peptides containing human sequence segments induced no T cell proliferation in any donor at any time point whilst each of the wild type peptides induced proliferation with SI>2 in >10% of all donors for at least one time point.

### EXAMPLE 9

### Construction of a Composite Hirudin

Composite variants of the thrombin inhibitor hirudin (derived from Hirudo medicinalis) were generated using methods described in WO2004113386 using the protein with SEQ ID No 14 in table 7 as wild type. The location of T cell epitopes in hirudin was tested by analysis of overlapping 15 mer peptides as in WO2004113386 and the peptide 27-41 CILGSDGEKNQCVTG was shown to give a significant T cell response with human PBMCs from 20 healthy donors. The human sequence segment KCRH from human melanoma-associated antigen (AAN40505.1) was used to replace the hirudin residues at 26-29 using overlap PCR as in example 8 resulting in a variant hirudin molecule with 28/29IL changed to 28/29RH which retained full activity of the wild type hirudin using assays described in WO2004113386. The modified peptide 27-41 CRHGSDGEKNQCVTG was tested together with the wild type peptide 27-41 CILGSDGEKNQCVTG in T cell assays as in example 8 demonstrating the loss of T cell epitope activity in the modified peptide.

### EXAMPLE 10

### Construction of Composite Human Anti-IgE Antibody With Tr Epitopes

VH and VL genes from the Composite Human Anti-IgE antibody of example 4 were cloned according to standard polymerase chain reaction (PCR) methods from Orlandi et al., ibid into separate plasmid vectors as templates for a VL- and VH-specific PCR using oligonucleotide primer pairs. Overlapping complementary sequences were introduced into the PCR products that combined during the subsequent fusion PCR to form the coding sequence either of a 20 amino acid (G₄S₁)₄ linker or, alternatively, a 20 amino acid sequence GGSNNLSCLTIPASANNGGS containing a 10 amino acid Tr epitope from the hepatitis C core protein (P19, MacDonald et al., Journal of Infectious Diseases, 185 (2002) p720-727) flanked each side by two asparagines residues and a GGS triplet. This final amplification step was performed with primer pairs for subsequent cleavage with the restriction enzymes EcoRV and BspE1 and cloning into the bluescript KS vector (Stratagene). Dimeric forms of the Composite Human anti-IgE single chain antibodies (scFvs) were then constructed by the method of Mack et al., Proc Natl Acad Sci USA., 92 (1995) p7021-7025. The dimeric VL-linker-VH-VL-linker-VH fragment was subcloned into the EcoR1/Sal1 sites of the expression vector pEF-DHFR (Mack et al., ibid) and transfected into DHFR-deficient Chinese hamster ovary (CHO) cells by electroporation. Selection, gene amplification, and protein production were performed as described by Mach et al., ibid). The dimeric scFv's were purified via the C-terminal histidine tails by affinity chromatography on a nickel-nitrilotriacetic acid (Ni-NTA) column (Qiagen) to give dimeric Fvs designated CHABIgEG4S1×4 ((G₄S₁)₄ linker between VL and VH) and CHABIgEHCVP19 (HCV Tr epitope between VL and VH).

Dimeric scFvs were subsequently tested in human T cell assays at 50 μg/ml exactly as described by Hall et al., Blood 100 (2002) p4529-4536 using PBMCs from 20 healthy donors. The results showed no significant proliferation of T cell for either CHABIgEG4S1×4 or CHABIgEHCVP19 but showed a significant level of IL-10 production (SI>2) from 4 out of 20 donors stimulated with CHABIgEHCVP19 but not with CHABIgEG4S1×4 (SI>2 in 0 of 20 donors). This demonstrates the effect of a Tr epitope included within the antibody molecule for the induction of the immunosuppressive cytokine IL-10.

1. A modified antibody or antigen-binding fragment thereof wherein the heavy and light chain variable regions of the modified antibody or antigen-binding fragment are each composed of two or more segments of amino acid sequence from one or more other antibodies or antigen-binding fragments, whereby the segments are neither whole CDRs nor framework regions.

2-36. (canceled)

37. A method for screening composite antibody variable regions comprising:
generating a library of genes encoding composite antibody variable regions derived from multiple segments of amino acid sequence of 2 to 31 amino acids long from other antibodies or antigen-binding fragments, wherein the multiple segments are neither whole CDRs nor whole framework regions;
screening the composite antibody variable regions to avoid T cell epitopes; and
expressing at least a portion of the library and screening the expressed antibody variable regions for binding to one or more antigens of interest.

38. The method of claim 37, wherein the multiple segments of amino acid sequence are derived from human antibodies.

39. The method of claim 37, wherein the expressed antibody variable regions form part of expressed antibodies.

40. The method of claim 37, wherein the expressed antibody variable regions form part of expressed antigen-binding fragments.

41. The method of claim 40, wherein the expressed antigen-binding fragments are selected from Fv's, Fab's, Fab2's, SCA's, single domain antibodies, and multimeric derivatives of each of these.

42. A method for producing copies of a composite antibody variable region of interest comprising:
generating a library of genes encoding composite antibody variable regions derived from multiple segments of amino acid sequence of 2 to 31 amino acids long from other antibodies or antigen-binding fragments, wherein the multiple segments are neither whole CDRs nor whole framework regions;
screening the composite antibody variable regions to avoid T cell epitopes;
expressing at least a portion of the library and screening the expressed antibody variable regions for binding to one or more antigens of interest;
identifying an antibody variable region of interest; and
producing additional copies of the identified antibody variable region of interest.

43. The method of claim 42, wherein the multiple segments of amino acid sequence are derived from human antibodies.

44. The method of claim 42, wherein the additional copies of the identified antibody variable region of interest form part of produced antibodies.

45. The method of claim 42, wherein the additional copies of the identified antibody variable region of interest form part of produced antigen-binding fragments.

46. The method of claim 45, wherein the produced antigen-binding fragments are selected from Fv's, Fab's, Fab2's, SCA's, single domain antibodies, and multimeric derivatives of each of these.

47. The method of claim 44, wherein the produced antibodies comprise one or more regulatory T cell epitopes which suppress immune reactions.

48. The method of claim 45, wherein the produced antigen-binding fragments comprise one or more regulatory T cell epitopes which suppress immune reactions.


SEQUENCE LISTING (600 sequences)

SEQ ID NO: 1 - protein, 120 aa, Homo sapiens
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSS

SEQ ID NO: 2 - protein, 107 aa, Homo sapiens
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 3 - protein, 120 aa, Homo sapiens
EVQLVESGGGLVQPGGSLKLSCVASGFIFSNHWMNWVRQAPGKGLEWVAEIRSKSINSATHYAESVKGRFTISRDDSKSIVYLQMTDLRTEDTGVYYCSRNYYGSTYDYWGQGTTVTVSS

SEQ ID NO: 4 - protein, 107 aa, Homo sapiens
DILLTQSPAILSLSPGERATLSCRASQFVGSSIHWYQQKTNQSPKLLIKYASESMSGIPSRFSGSGSGTDFTLTINSLESEDAADYYCQQSHSWPFTFGQGTNLEIK

SEQ ID NO: 5 - protein, 120 aa, Homo sapiens
EVQLVESGGGLVQPGGSLKLSCVASGFIFSNHWMNWVRQAPGKGLEWVAEIRSKSINSATHYAESVKGRFTISRDDSKSIVYLQMNDLKTEDTGVYYCSRNYYGSTYDYWGQGTTVTVSS

SEQ ID NO: 6 - protein, 120 aa, Homo sapiens
EVQLVESGGGLVQPGGSLKLSCVASGFIFSNHWMNWVRQAPGKGLEWVAEIRSKSINSATHYAESVKGRFTISRDDSKSIVYLQMNSLKTEDTGVYYCSRNYYGSTYDYWGQGTTVTVSS

SEQ ID NO: 7 - protein, 120 aa, Homo sapiens
EVQLVESGGGLVQPGGSLKLSCAASGFIFSNHWMNWVRQAPGKGLEWVAEIRSKSINSATHYAESVKGRFTISRDDSKSIVYLQMNSLKTEDTGVYYCSRNYYGSTYDYWGQGTTVTVSS

SEQ ID NO: 8 - protein, 120 aa, Homo sapiens
EVQLVESGGGLVQPGGSLKLSCAASGFIFSNHWMNWVRQAPGKGLEWVAEIRSKSINSATHYAESVKGRFTISRDDSKSIAYLQMNSLKTEDTGVYYCSRNYYGSTYDYWGQGTTVTVSS

SEQ ID NO: 9 - protein, 107 aa, Homo sapiens
DILLTQSPATLSLSPGERATLSCRASQFVGSSIHWYQQKTNQSPKLLIKYASESMSGIPSRFSGSGSGTDFTLTINSLESEDAADYYCQQSHSWPFTFGQGTRLEIK

SEQ ID NO: 10 - protein, 107 aa, Homo sapiens
DILLTQSPATLSLSPGERATLSCRASQFVGSSIHWYQQKTNQSPKLLIKYASESMSGIPSRFSGSGSGTDFTLTINSLEAEDAADYYCQQSHSWPFTFGQGTRLEIK

SEQ ID NO: 11 - protein, 107 aa, Homo sapiens
DILLTQSPATLSLSPGERATLSCRASQFVGSSIHWYQQKTDQSPKLLIKYASESMSGIPSRFSGSGSGTDFTLTINSLEAEDAADYYCQQSHSWPFTFGQGTRLEIK

SEQ ID NO: 12 - DNA, 38 bp, Homo sapiens
gttgctacgcgtgtccactccgaagtgaagcttgagga

SEQ ID NO: 13 - DNA, 40 bp, Homo sapiens
gtctggaggaggcttggtgcaacctggaggatccatgaaa

SEQ ID NO: 14 - DNA, 40 bp, Homo sapiens
ctctcctgtgttgcctctggattcattttcagtaaccact

SEQ ID NO: 15 - DNA, 40 bp, Homo sapiens
ggatgaactgggtccgccagtctccagagaaggggcttga

SEQ ID NO: 16 - DNA, 40 bp, Homo sapiens
gtgggttgctgaaattagatcgaaatctattaattctgca

SEQ ID NO: 17 - DNA, 40 bp, Homo sapiens
acacattatgcggagtctgtgaaagggaggttcaccatct

SEQ ID NO: 18 - DNA, 40 bp, Homo sapiens
caagagatgattccaaaagtgctgtctacctgcaaatgac

SEQ ID NO: 19 - DNA, 40 bp, Homo sapiens
cgacttaagaactgaagacactggcgtttattactgtagc

SEQ ID NO: 20 - DNA, 40 bp, Homo sapiens
aggaactattacggttcaacctacgactactggggccaag

SEQ ID NO: 21 - DNA, 38 bp, Homo sapiens
gcaccactctcacagtctcctcaggtaagctttctggg

SEQ ID NO: 22 - DNA, 18 bp, Homo sapiens
cccagaaagcttacctga

SEQ ID NO: 23 - DNA, 40 bp, Homo sapiens
ggagactgtgagagtggtgccttggccccagtagtcgtag

SEQ ID NO: 24 - DNA, 40 bp, Homo sapiens
gttgaaccgtaatagttcctgctacagtaataaacgccag

SEQ ID NO: 25 - DNA, 40 bp, Homo sapiens
tgtcttcagttcttaagtcggtcatttgcaggtagacagc

SEQ ID NO: 26 - DNA, 40 bp, Homo sapiens
acttttggaatcatctcttgagatggtgaacctccctttc

SEQ ID NO: 27 - DNA, 40 bp, Homo sapiens
acagactccgcataatgtgttgcagaattaatagatttcg

SEQ ID NO: 28 - DNA, 40 bp, Homo sapiens
atctaatttcagcaacccactcaagccccttctctggaga

SEQ ID NO: 29 - DNA, 40 bp, Homo sapiens
ctggcggacccagttcatccagtggttactgaaaatgaat

SEQ ID NO: 30 - DNA, 40 bp, Homo sapiens
ccagaggcaacacaggagagtttcatggatcctccaggtt

SEQ ID NO: 31 - DNA, 40 bp, Homo sapiens
gcaccaagcctcctccagactcctcaagcttcacttcgga

SEQ ID NO: 32 - DNA, 18 bp, Homo sapiens
gtggacacgcgtagcaac

SEQ ID NO: 33 - DNA, 40 bp, Homo sapiens
tcccaggcgcgcgatgtgacatcctgctgacacaatctcc

SEQ ID NO: 34 - DNA, 40 bp, Homo sapiens
agccatcctgtctgtgagtccaggagaaagagtcagtttc

SEQ ID NO: 35 - DNA, 40 bp, Homo sapiens
tcctgcagggccagtcagttcgttggctcaagcatacact

SEQ ID NO: 36 - DNA, 40 bp, Homo sapiens
ggtatcagcagagaacaaatggttctccaaggcttctcat

SEQ ID NO: 37 - DNA, 40 bp, Homo sapiens
aaagtatgcttctgagtctatgtctgggatctcttccagg

SEQ ID NO: 38 - DNA, 40 bp, Homo sapiens
tttagtggcagtggatcagggacagattttactcttagca

SEQ ID NO: 39 - DNA, 40 bp, Homo sapiens
tcaacactgtggagtctgaagatattgcagattattactg

SEQ ID NO: 40 - DNA, 40 bp, Homo sapiens
tcaacaaagtcatagttggccgttcacgttcggttctggg

SEQ ID NO: 41 - DNA, 40 bp, Homo sapiens
acaaatttggaagtaaaacgtgagtagaatttaaactttg

SEQ ID NO: 42 - DNA, 23 bp, Homo sapiens
cttcctcagttggatcctggcag

SEQ ID NO: 43 - DNA, 43 bp, Homo sapiens
ctgccaggatccaactgaggaagcaaagtttaaattctactca

SEQ ID NO: 44 - DNA, 40 bp, Homo sapiens
cgttttacttccaaatttgtcccagaaccgaacgtgaacg

SEQ ID NO: 45 - DNA, 40 bp, Homo sapiens
gccaactatgactttgttgacagtaataatctgcaatatc

SEQ ID NO: 46 - DNA, 40 bp, Homo sapiens
ttcagactccacagtgttgatgctaagagtaaaatctgtc

SEQ ID NO: 47 - DNA, 40 bp, Homo sapiens
cctgatccactgccactaaacctggaagagatcccagaca

SEQ ID NO: 48 - DNA, 40 bp, Homo sapiens
tagactcagaagcatactttatgagaagccttggagaacc

SEQ ID NO: 49 - DNA, 40 bp, Homo sapiens
atttgttctctgctgataccagtgtatgcttgagccaacg

SEQ ID NO: 50 - DNA, 40 bp, Homo sapiens
aactgactggccctgcaggagaaactgactctttctcctg

SEQ ID NO: 51 - DNA, 40 bp, Homo sapiens
gactcacagacaggatggctggagattgtgtcagcaggat

SEQ ID NO: 52 - DNA, 20 bp, Homo sapiens
gtcacatcgcgcgcctggga

SEQ ID NO: 53 - DNA, 38 bp, Homo sapiens
gttgctacgcgtgtccactccgaagtgcagcttgtgga

SEQ ID NO: 54 - DNA, 40 bp, Homo sapiens
gtctggaggaggcttggtgcaacctggaggatccttgaaa

SEQ ID NO: 55 - DNA, 40 bp, Homo sapiens; identical to SEQ ID NO: 14

SEQ ID NO: 56 - DNA, 40 bp, Homo sapiens
ggatgaactgggtccgccaggctccagggaaggggcttga

SEQ ID NO: 57 - DNA, 40 bp, Homo sapiens; identical to SEQ ID NO: 16

SEQ ID NO: 58 - DNA, 40 bp, Homo sapiens; identical to SEQ ID NO: 17

SEQ ID NO: 59 - DNA, 40 bp, Homo sapiens
caagagatgattccaaaagtattgtctacctgcaaatgac

SEQ ID NO: 60 - DNA, 40 bp, Homo sapiens; identical to SEQ ID NO: 19

SEQ ID NO: 61 - DNA, 40 bp, Homo sapiens; identical to SEQ ID NO: 20

SEQ ID NO: 62 - DNA, 38 bp, Homo sapiens
gcaccactgtcacagtctcctcaggtaagctttctggg

SEQ ID NO: 63 - DNA, 18 bp, Homo sapiens
cccagaaagcttacctga

SEQ ID NO: 64 - DNA, 40 bp, Homo sapiens
ggagactgtgacagtggtgccttggccccagtagtcgtag

SEQ ID NO: 65 - DNA, 40 bp, Homo sapiens; identical to SEQ ID NO: 24

SEQ ID NO: 66 - DNA, 40 bp, Homo sapiens
tgtcttcagttcttaagtcggtcatttgcaggtagacaat

SEQ ID NO: 67 - DNA, 40 bp, Homo sapiens
cacttttggaatcatctcttgagatggtgaacctcctttc

SEQ ID NO: 68 - DNA, 40 bp, Homo sapiens; identical to SEQ ID NO: 27

SEQ ID NO: 69 - DNA, 40 bp, Homo sapiens
atctaatttcagcaacccactcaagccccttccctggagc

SEQ ID NO: 70 - DNA, 40 bp, Homo sapiens; identical to SEQ ID NO: 29

SEQ ID NO: 71 - DNA, 40 bp, Homo sapiens
ccagaggcaacacaggagagtttcaaggatcctccaggtt

SEQ ID NO: 72 - DNA, 40 bp, Homo sapiens
gcaccaagcctcctccagactccacaagctgcacttcgga

SEQ ID NO: 73 - DNA, 18 bp, Homo sapiens
gtggacacgcgtagcaac

SEQ ID NO: 74 - DNA, 40 bp, Homo sapiens; identical to SEQ ID NO: 33

SEQ ID NO: 75 - DNA, 40 bp, Homo sapiens
agccatcctgtctctgagtccaggagaaagagccactctc

SEQ ID NO: 76 - DNA, 40 bp, Homo sapiens; identical to SEQ ID NO: 35

SEQ ID NO: 77 - DNA, 40 bp, Homo sapiens
ggtatcagcagaaaacaaatcagtctccaaagcttctcat

SEQ ID NO: 78 - DNA, 40 bp, Homo sapiens; identical to SEQ ID NO: 37

SEQ ID NO: 79 - DNA, 40 bp, Homo sapiens
tttagtggcagtggatcagggacagattttactcttacca

SEQ ID NO: 80 - DNA, 40 bp, Homo sapiens
tcaacagtctggagtctgaagatgctgcagattattactg

SEQ ID NO: 81 - DNA, 40 bp, Homo sapiens
tcaacaaagtcatagttggccgttcacgttcggtcaaggg

SEQ ID NO: 82 - DNA, 40 bp, Homo sapiens
acaaatttggaaataaaacgtgagtagaatttaaactttg

SEQ ID NO: 83 - DNA, 23 bp, Homo sapiens
cttcctcagttggatcctggcag

SEQ ID NO: 84 - DNA, 43 bp, Homo sapiens; identical to SEQ ID NO: 43

SEQ ID NO: 85 - DNA, 40 bp, Homo sapiens
cgttttatttccaaatttgtcccttgaccgaacgtgaacg

SEQ ID NO: 86 - DNA, 40 bp, Homo sapiens
gccaactatgactttgttgacagtaataatctgcagcatc

SEQ ID NO: 87 - DNA, 40 bp, Homo sapiens
ttcagactccagactgttgatggtaagagtaaaatctgtc

SEQ ID NO: 88 - DNA, 40 bp, Homo sapiens; identical to SEQ ID NO: 47

SEQ ID NO: 89 - DNA, 40 bp, Homo sapiens
tagactcagaagcatactttatgagaagctttggagactg

SEQ ID NO: 90 - DNA, 40 bp, Homo sapiens
atttgttttctgctgataccagtgtatgcttgagccaacg

SEQ ID NO: 91 - DNA, 40 bp, Homo sapiens
aactgactggccctgcaggagagagtggctctttctcctg

SEQ ID NO: 92 - DNA, 40 bp, Homo sapiens
gactcagagacaggatggctggagattgtgtcagcaggat

SEQ ID NO: 93 - DNA, 20 bp, Homo sapiens
gtcacatcgcgcgcctggga

SEQ ID NO: 94 - DNA, 25 bp, Homo sapiens
caaatgaacgacttaaaaactgaag

SEQ ID NO: 95 - DNA, 25 bp, Homo sapiens
cttcagtttttaagtcgttcatttg

SEQ ID NO: 96 - DNA, 25 bp, Homo sapiens
caaatgaacagcttaaaaactgaag

SEQ ID NO: 97 - DNA, 25 bp, Homo sapiens
cttcagtttttaagctgttcatttg

SEQ ID NO: 98 - DNA, 20 bp, Homo sapiens
ctctcctgtgctgcctctgg

SEQ ID NO: 99 - DNA, 20 bp, Homo sapiens
ccagaggcagcacaggagag

SEQ ID NO: 100 - DNA, 20 bp, Homo sapiens
caaaagtattgcctacctgc

SEQ ID NO: 101 - DNA, 20 bp, Homo sapiens
cgaggtaggcaatacttttg

SEQ ID NO: 102 - DNA, 54 bp, Homo sapiens
tcccaggcgcgcgatgtgacatcctgctgacacaatctccagccaccctgtctc

SEQ ID NO: 103 - DNA, 66 bp, Homo sapiens
ctgccaggatccaactgaggaagcaaagtttaaattctactcacgttttatttccaatcttgtccc

SEQ ID NO: 104 - DNA, 21 bp, Homo sapiens
cagtctggaggctgaagatgc

SEQ ID NO: 105 - DNA, 21 bp, Homo sapiens
cgatcttcagcctccagactg

SEQ ID NO: 106 - DNA, 21 bp, Homo sapiens
gcagaaaacagatcagtctcc

SEQ ID NO: 107 - DNA, 21 bp, Homo sapiens
ggagactgatctgttttctga

SEQ ID NO: 108 - protein, 9 aa, Homo sapiens
DFLSGYLDY

SEQ ID NO: 109 - protein, 9 aa, Homo sapiens
VRGSGSFDY

SEQ ID NO: 110 - protein, 9 aa, Homo sapiens
DRGGNYFDY

SEQ ID NO: 111 - protein, 9 aa, Homo sapiens
MYNWNFFDY

SEQ ID NO: 112 - protein, 9 aa, Homo sapiens
AGLGMIFDY

SEQ ID NO: 113 - protein, 9 aa, Homo sapiens
RGFNGQLIF

SEQ ID NO: 114 - protein, 9 aa, Homo sapiens
ALTGDAFDI

SEQ ID NO: 115 - protein, 9 aa, Homo sapiens
TKLDWYFDL

SEQ ID NO: 116 - protein, 9 aa, Homo sapiens
RYGGFYFDY

SEQ ID NO: 117 - protein, 9 aa, Homo sapiens
GYSNEGMDV

SEQ ID NO: 118 - protein, 9 aa, Homo sapiens
SWDGYSYIY

SEQ ID NO: 119 - protein, 9 aa, Homo sapiens
QMGAEYFQH

SEQ ID NO: 120 - protein, 9 aa, Homo sapiens
DMSLDAFDI

SEQ ID NO: 121 - protein, 9 aa, Homo sapiens
GSVGATLGE

SEQ ID NO: 122 - protein, 9 aa, Homo sapiens
YGDYHYFDY

SEQ ID NO: 123 - protein, 9 aa, Homo sapiens
GVGSSGWDH

SEQ ID NO: 124 - protein, 9 aa, Homo sapiens
KGSLYYFDY

SEQ ID NO: 125 - protein, 9 aa, Homo sapiens
PNWNDAFDI

SEQ ID NO: 126 - protein, 9 aa, Homo sapiens
RGIPHAFDI

SEQ ID NO: 127 - protein, 9 aa, Homo sapiens
PPEVESLRS

SEQ ID NO: 128 - protein, 9 aa, Homo sapiens
PPEVESLRS

SEQ ID NO: 129 - protein, 9 aa, Homo sapiens
PPEVESLRS

SEQ ID NO: 130 - protein, 9 aa, Homo sapiens
PPEVESLRS

SEQ ID NO: 131 - protein, 9 aa, Homo sapiens
PPEVESLRS

SEQ ID NO: 132 - protein, 9 aa, Homo sapiens
PPEVESLRS

SEQ ID NO: 133 - protein, 9 aa, Homo sapiens
PPEVESLRS

SEQ ID NO: 134 - protein, 9 aa, Homo sapiens
PPEVESLRS

SEQ ID NO: 135 - protein, 8 aa, Homo sapiens
DLAAARLF

SEQ ID NO: 136 - protein, 9 aa, Homo sapiens
QGTIAGIRH

SEQ ID NO: 137 - protein, 9 aa, Homo sapiens
EDYYYGMDV

SEQ ID NO: 138 - protein, 9 aa, Homo sapiens
DPINWYFDL

SEQ ID NO: 139 - protein, 9 aa, Homo sapiens
DRAAGDRDY

SEQ ID NO: 140 - protein, 9 aa, Homo sapiens
HQMYSNSDY

SEQ ID NO: 141 - protein, 9 aa, Homo sapiens
SYDFAWFAY

SEQ ID NO: 142 - protein, 9 aa, Homo sapiens
QGTIAGIRH

SEQ ID NO: 143 - protein, 9 aa, Homo sapiens
VLGIIAADH

SEQ ID NO: 144 - protein, 9 aa, Homo sapiens
DLTGDAFDI

SEQ ID NO: 145 - protein, 9 aa, Homo sapiens
SCGSQYFDY

SEQ ID NO: 146 - protein, 9 aa, Homo sapiens
LWNWDAFDI

SEQ ID NO: 147 - protein, 9 aa, Homo sapiens
DIMTWGFDY

SEQ ID NO: 148 - protein, 9 aa, Homo sapiens
SNWYWYFDL

SEQ ID NO: 149 - protein, 9 aa, Homo sapiens
NLIAGCIDV

SEQ ID NO: 150 - protein, 9 aa, Homo sapiens
GGKGGEFDD

SEQ ID NO: 151 - protein, 9 aa, Homo sapiens
DSGNYRIDY

SEQ ID NO: 152 - protein, 9 aa, Homo sapiens
DPRLDAFDI

SEQ ID NO: 153 - protein, 9 aa, Homo sapiens
GYSYPVWGR

SEQ ID NO: 154 - protein, 9 aa, Homo sapiens
LVGNSWLDY

SEQ ID NO: 155 - protein, 9 aa, Homo sapiens
  Unknown (3)..(3)
  misc_feature (3)..(3): Xaa can be any naturally occurring amino acid
DLXGLVVEY

SEQ ID NO: 156 - protein, 9 aa, Homo sapiens
KVSLSAFDI

SEQ ID NO: 157 - protein, 9 aa, Homo sapiens
RGDAMYFDV

SEQ ID NO: 158 - protein, 9 aa, Homo sapiens
DPNPWYFDL

SEQ ID NO: 159 - protein, 9 aa, Homo sapiens
DYGDYAFDI

SEQ ID NO: 160 - protein, 9 aa, Homo sapiens
SAHSDAFDM

SEQ ID NO: 161 - protein, 9 aa, Homo sapiens
LEGLGWFDP

SEQ ID NO: 162 - protein, 9 aa, Homo sapiens
RSDYGAIDY

SEQ ID NO: 163 - protein, 9 aa, Homo sapiens
NLGFYHMDV

SEQ ID NO: 164 - protein, 9 aa, Homo sapiens
EARGGGGEY

SEQ ID NO: 165 - protein, 9 aa, Homo sapiens
EGWISALNG

SEQ ID NO: 166 - protein, 9 aa, Homo sapiens
EGEGEYFDY

SEQ ID NO: 167 - protein, 9 aa, Homo sapiens
ERTSGDFDF

SEQ ID NO: 168 - protein, 9 aa, Homo sapiens
NSPGATFES

SEQ ID NO: 169 - protein, 9 aa, Homo sapiens
GNGQKCFDY

SEQ ID NO: 170 - protein, 9 aa, Homo sapiens
RGSLQYLDY

SEQ ID NO: 171 - protein, 9 aa, Homo sapiens
NNGSYYFDY

SEQ ID NO: 172 - protein, 9 aa, Homo sapiens
GSDYSNFAY

SEQ ID NO: 173 - protein, 9 aa, Homo sapiens
STHRSAFDV

SEQ ID NO: 174 - protein, 9 aa, Homo sapiens
EGVHKNFDH

SEQ ID NO: 175 - protein, 9 aa, Homo sapiens
LSRAGGFDI

SEQ ID NO: 176 - protein, 9 aa, Homo sapiens
RMPAVAFDY

SEQ ID NO: 177 - protein, 9 aa, Homo sapiens
RMPAVAFDY

SEQ ID NO: 178 - protein, 9 aa, Homo sapiens
RMRAVAFDY

SEQ ID NO: 179 - protein, 9 aa, Homo sapiens
RMPAVAFDY

SEQ ID NO: 180 - protein, 9 aa, Homo sapiens
DYGGNPAEL

SEQ ID NO: 181 - protein, 9 aa, Homo sapiens
GPTCSGGSC

SEQ ID NO: 182 - protein, 9 aa, Homo sapiens
RKGAAHFDY

SEQ ID NO: 183 - protein, 9 aa, Homo sapiens
EEVGGYFQH

SEQ ID NO: 184 - protein, 9 aa, Homo sapiens
DFDGGSFDY

SEQ ID NO: 185 - protein, 9 aa, Homo sapiens
DFDGGSLDY

SEQ ID NO: 186 - protein, 9 aa, Homo sapiens
DFDGGSFDY

SEQ ID NO: 187 - protein, 9 aa, Homo sapiens
KVPSHGMDY

SEQ ID NO: 188 - protein, 9 aa, Homo sapiens
KVPSHGMDY

SEQ ID NO: 189 - protein, 9 aa, Homo sapiens
KVPSHGMDY

SEQ ID NO: 190 - protein, 9 aa, Homo sapiens
KVPSHGMDY

SEQ ID NO: 191 - protein, 9 aa, Homo sapiens
QPLARHFDP

SEQ ID NO: 192 - protein, 9 aa, Homo sapiens
GPLMRWFDD

SEQ ID NO: 193 - protein, 9 aa, Homo sapiens
VAVAGGFDP

SEQ ID NO: 194 - protein, 9 aa, Homo sapiens
GVEVAGTAS

SEQ ID NO: 195 - protein, 9 aa, Homo sapiens
YYESSAGPP

SEQ ID NO: 196 - protein, 9 aa, Homo sapiens
EIPRGGSCY

SEQ ID NO: 197 - protein, 9 aa, Homo sapiens
EIPRGGSCY

SEQ ID NO: 198 - protein, 9 aa, Homo sapiens
KEKWDSSRC

SEQ ID NO: 199 - protein, 9 aa, Homo sapiens
GSAAAGTQG

SEQ ID NO: 200 - protein, 9 aa, Homo sapiens
DFSWAGPHF

SEQ ID NO: 201 - protein, 9 aa, Homo sapiens
GTHYYDIRV

SEQ ID NO: 202 - protein, 9 aa, Homo sapiens
DGSGSYEGN

SEQ ID NO: 203 - protein, 9 aa, Homo sapiens
GGAVAAFDY

SEQ ID NO: 204 - protein, 9 aa, Homo sapiens
KPVTGGEDY

SEQ ID NO: 205 - protein, 9 aa, Homo sapiens
DYDGAWFAY

SEQ ID NO: 206 - protein, 9 aa, Homo sapiens
WDGRLLVDY

SEQ ID NO: 207 - protein, 9 aa, Homo sapiens
HKGLRYFDY

SEQ ID NO: 208 - protein, 9 aa, Homo sapiens
HKGLRYFDY

SEQ ID NO: 209 - protein, 9 aa, Homo sapiens
HKGLRYFDY

SEQ ID NO: 210 - protein, 9 aa, Homo sapiens
HKGLRYFDY

SEQ ID NO: 211 - protein, 9 aa, Homo sapiens
HKGLRYFDY

SEQ ID NO: 212 - protein, 9 aa, Homo sapiens
HKGLRYFDY

SEQ ID NO: 213 - protein, 9 aa, Homo sapiens
YRGDTYDYS

SEQ ID NO: 214 - protein, 9 aa, Homo sapiens
WVGATTSDY

SEQ ID NO: 215 - protein, 9 aa, Homo sapiens
EDMDYGMDV

SEQ ID NO: 216 - protein, 9 aa, Homo sapiens
GGRDRYLVY

SEQ ID NO: 217 - protein, 9 aa, Homo sapiens
VRVSYGMDV

SEQ ID NO: 218 - protein, 9 aa, Homo sapiens
MRKGYAMDY

SEQ ID NO: 219 - protein, 9 aa, Homo sapiens
MRKGYAMDY

SEQ ID NO: 220 - protein, 9 aa, Homo sapiens
MRKGYAMDY

SEQ ID NO: 221 - protein, 9 aa, Homo sapiens
MRKGYAMDY

SEQ ID NO: 222 - protein, 9 aa, Homo sapiens
MRKGYAMDY

SEQ ID NO: 223 - protein, 9 aa, Homo sapiens
RMPAVAFDY

SEQ ID NO: 224 - protein, 9 aa, Homo sapiens
RMPAVAFDY

SEQ ID NO: 225 - protein, 9 aa, Homo sapiens
RMRAVAFDY

SEQ ID NO: 226 - protein, 9 aa, Homo sapiens
RMPAVAFDY

SEQ ID NO: 227 - protein, 9 aa, Homo sapiens
TSIVRGFGP

SEQ ID NO: 228 - protein, 9 aa, Homo sapiens
DFFRDYFDY

SEQ ID NO: 229 - protein, 9 aa, Homo sapiens
DFFRDYFDY

SEQ ID NO: 230 - protein, 9 aa, Homo sapiens
GGTQPFDIR

SEQ ID NO: 231 - protein, 9 aa, Homo sapiens
SQASGPFDY

SEQ ID NO: 232 - protein, 9 aa, Homo sapiens
GLYQLLFDY

SEQ ID NO: 233 - protein, 9 aa, Homo sapiens
AGGRTSFDP

SEQ ID NO: 234 - protein, 9 aa, Homo sapiens
EGNTKAPDY

SEQ ID NO: 235 - protein, 9 aa, Homo sapiens
NGTSGDFDY

SEQ ID NO: 236 - protein, 9 aa, Homo sapiens
YGTSYWFPY

SEQ ID NO: 237 - protein, 9 aa, Homo sapiens
YGTSYWFPY

SEQ ID NO: 238 - protein, 9 aa, Homo sapiens
GGRDRYLVY

SEQ ID NO: 239 - protein, 9 aa, Homo sapiens
LRYQLLYNY

SEQ ID NO: 240 - protein, 9 aa, Homo sapiens
YIAYDAFDI

SEQ ID NO: 241 - protein, 9 aa, Homo sapiens
ITPRNAVDI

SEQ ID NO: 242 - protein, 9 aa, Homo sapiens
DGLLAATDY

SEQ ID NO: 243 - protein, 9 aa, Homo sapiens
DRAYLDFWG

SEQ ID NO: 244 - protein, 9 aa, Homo sapiens
DKEPAYFDY

SEQ ID NO: 245 - protein, 9 aa, Homo sapiens
RGFNGQLIF

SEQ ID NO: 246 - protein, 9 aa, Homo sapiens
LSVVVPAAL

SEQ ID NO: 247 - protein, 9 aa, Homo sapiens
LADDDPEDF

SEQ ID NO: 248 - protein, 9 aa, Homo sapiens
EDMDYGMDV

SEQ ID NO: 249 - protein, 9 aa, Homo sapiens
SAGGSAWST

SEQ ID NO: 250 - protein, 9 aa, Homo sapiens
DRSYYGMDV

SEQ ID NO: 251 - protein, 9 aa, Homo sapiens
DKGTRYSDQ

SEQ ID NO: 252 - protein, 9 aa, Homo sapiens
WLVEGSFDY

SEQ ID NO: 253 - protein, 9 aa, Homo sapiens
GYVGSSLDY

SEQ ID NO: 254 - protein, 9 aa, Homo sapiens
WHQLRGPDY

SEQ ID NO: 255 - protein, 9 aa, Homo sapiens
ENSDYYFDY

SEQ ID NO: 256 - protein, 9 aa, Homo sapiens
DGTYGSGVR

SEQ ID NO: 257 - protein, 9 aa, Homo sapiens
GGSMVPFDY

SEQ ID NO: 258 - protein, 9 aa, Homo sapiens
RGWNYYFDS

SEQ ID NO: 259 - protein, 9 aa, Homo sapiens
DAYYYGLDV

SEQ ID NO: 260 - protein, 9 aa, Homo sapiens
DGRYDPIDY

SEQ ID NO: 261 - protein, 9 aa, Homo sapiens
VGSSGWYDY

SEQ ID NO: 262 - protein, 9 aa, Homo sapiens
DLYDYYDEP

SEQ ID NO: 263 - protein, 9 aa, Homo sapiens
DGAAASFDY

SEQ ID NO: 264 - protein, 9 aa, Homo sapiens
VVGADYFDY

SEQ ID NO: 265 - protein, 9 aa, Homo sapiens
DQNWGYFDY

SEQ ID NO: 266 - protein, 9 aa, Homo sapiens
GVLRDAFDI

SEQ ID NO: 267 - protein, 9 aa, Homo sapiens
ASDGYGMDV

SEQ ID NO: 268 - protein, 9 aa, Homo sapiens
GVLRHALDI

SEQ ID NO: 269 - protein, 9 aa, Homo sapiens
GGCGWYKNY

SEQ ID NO: 270 - protein, 9 aa, Homo sapiens
GSNYAKTGY

SEQ ID NO: 271 - protein, 9 aa, Homo sapiens
GKFQLLFDY

SEQ ID NO: 272 - protein, 9 aa, Homo sapiens
ALHGGGMDV

SEQ ID NO: 273 - protein, 9 aa, Homo sapiens
ALHGGGMDV

SEQ ID NO: 274 - protein, 9 aa, Homo sapiens
VYPPDAFDL

SEQ ID NO: 275 - protein, 9 aa, Homo sapiens
PWDYWFFDL

SEQ ID NO: 276 - protein, 9 aa, Homo sapiens
DRVAAAGDY

SEQ ID NO: 277 - protein, 9 aa, Homo sapiens
DKGTRYSDQ

SEQ ID NO: 278 - protein, 9 aa, Homo sapiens
DRVATIPDY

SEQ ID NO: 279 - protein, 9 aa, Homo sapiens
ERGITLMDV

SEQ ID NO: 280 - protein, 9 aa, Homo sapiens
ERGITLMDV

SEQ ID NO: 281 - protein, 9 aa, Homo sapiens
LDWLLPIDY

SEQ ID NO: 282 - protein, 9 aa, Homo sapiens
LDWLLPIDY

SEQ ID NO: 283 - protein, 9 aa, Homo sapiens
DDGDRAFGY

SEQ ID NO: 284 - protein, 9 aa, Homo sapiens
DPWPAAFDI

SEQ ID NO: 285 - protein, 9 aa, Homo sapiens
VRGSWSGDS

SEQ ID NO: 286 - protein, 9 aa, Homo sapiens
RHSSDWYPY

SEQ ID NO: 287 - protein, 9 aa, Homo sapiens
SSPYGALDY

SEQ ID NO: 288 - protein, 9 aa, Homo sapiens
GLDQYKTGH

SEQ ID NO: 289 - protein, 9 aa, Homo sapiens
GAGAAPHDY

SEQ ID NO: 290 - protein, 9 aa, Homo sapiens
GAGAAPHDY

SEQ ID NO: 291 - protein, 9 aa, Homo sapiens
NGTSGDFDY

SEQ ID NO: 292 - protein, 9 aa, Homo sapiens
ALRPATFDF

SEQ ID NO: 293 - protein, 8 aa, Homo sapiens
QQYADLIT

SEQ ID NO: 294 - protein, 8 aa, Homo sapiens
QQYYSTPT

SEQ ID NO: 295 - protein, 8 aa, Homo sapiens
QQYNTYPT

SEQ ID NO: 296 - protein, 8 aa, Homo sapiens
QQGNSFPK

SEQ ID NO: 297 - protein, 8 aa, Homo sapiens
QQYGYSLT

SEQ ID NO: 298 - protein, 8 aa, Homo sapiens
QQFGGSFT

SEQ ID NO: 299 - protein, 8 aa, Homo sapiens
QQSSNTVT

SEQ ID NO: 300 - protein, 8 aa, Homo sapiens
QQYNSLIT

SEQ ID NO: 301 - protein, 8 aa, Homo sapiens
QQYNNWPT

SEQ ID NO: 302 - protein, 8 aa, Homo sapiens
LQHNSYPF

SEQ ID NO: 303 - protein, 8 aa, Homo sapiens
QQYNSQYT

SEQ ID NO: 304 - protein, 8 aa, Homo sapiens
QQYGSLWT

SEQ ID NO: 305 - protein, 8 aa, Homo sapiens
QHYNRPWT

SEQ ID NO: 306 - protein, 8 aa, Homo sapiens
QQYGSRLT

SEQ ID NO: 307 - protein, 8 aa, Homo sapiens
QHYGTPRT

SEQ ID NO: 308 - protein, 8 aa, Homo sapiens
QQYNNWPT

SEQ ID NO: 309 - protein, 8 aa, Homo sapiens
MQATQFPT

SEQ ID NO: 310 - protein, 8 aa, Homo sapiens
HQASTYPL

SEQ ID NO: 311 - protein, 8 aa, Homo sapiens
QQYGRSPR

SEQ ID NO: 312 - protein, 8 aa, Homo sapiens
QQDDLPYT

SEQ ID NO: 313 - protein, 8 aa, Homo sapiens
QNDNLPLT

SEQ ID NO: 314 - protein, 8 aa, Homo sapiens
QQESLPLT

SEQ ID NO: 315 - protein, 8 aa, Homo sapiens
QQDNLPLT

SEQ ID NO: 316 - protein, 8 aa, Homo sapiens
QQESLPCG

SEQ ID NO: 317 - protein, 8 aa, Homo sapiens
QQDSLPLT

SEQ ID NO: 318 - protein, 8 aa, Homo sapiens
QQYGSSRS

SEQ ID NO: 319 - protein, 8 aa, Homo sapiens
QQYGSSRT

SEQ ID NO: 320 - protein, 8 aa, Homo sapiens
QQYCGSLS

SEQ ID NO: 321 - protein, 8 aa, Homo sapiens
QQSYSTLT

SEQ ID NO: 322 - protein, 8 aa, Homo sapiens
QLYGSSLT

SEQ ID NO: 323 - protein, 8 aa, Homo sapiens
QQYNNLWT

SEQ ID NO: 324 - protein, 8 aa, Homo sapiens
QQYNTFFT

SEQ ID NO: 325 - protein, 8 aa, Homo sapiens
QQYGSSPT

SEQ ID NO: 326 - protein, 8 aa, Homo sapiens
QQYGSSLT

SEQ ID NO: 327 - protein, 8 aa, Homo sapiens
QQYGSSLT

SEQ ID NO: 328 - protein, 8 aa, Homo sapiens
QQYGSSKT

SEQ ID NO: 329 - protein, 8 aa, Homo sapiens
QQYNNWPP

SEQ ID NO: 330 - protein, 8 aa, Homo sapiens
QHRNNWPP

SEQ ID NO: 331 - protein, 8 aa, Homo sapiens
QQRSNWPS

SEQ ID NO: 332 - protein, 8 aa, Homo sapiens
QQYGSSPT

SEQ ID NO: 333 - protein, 8 aa, Homo sapiens
QQYDTIPT

SEQ ID NO: 334 - protein, 8 aa, Homo sapiens
QASINTPL

SEQ ID NO: 335 - protein, 8 aa, Homo sapiens
MQALQPWT

SEQ ID NO: 336 - protein, 8 aa, Homo sapiens
QQGFSDRS

SEQ ID NO: 337 - protein, 8 aa, Homo sapiens
MQATQFVT

SEQ ID NO: 338 - protein, 8 aa, Homo sapiens
QRCKGMFS

SEQ ID NO: 339 - protein, 8 aa, Homo sapiens
QQYGGSPW

SEQ ID NO: 340 - protein, 8 aa, Homo sapiens
CRSHWPYT

SEQ ID NO: 341 - protein, 8 aa, Homo sapiens
QQYYSTPP

SEQ ID NO: 342 - protein, 8 aa, Homo sapiens
QQCNTNPP

SEQ ID NO: 343 - protein, 8 aa, Homo sapiens
QQYYSTPP

SEQ ID NO: 344 - protein, 8 aa, Homo sapiens
QQYYSVPP

SEQ ID NO: 345 - protein, 8 aa, Homo sapiens
QQYDSLVT

SEQ ID NO: 346 - protein, 8 aa, Homo sapiens
HQYLSSWT

SEQ ID NO: 347 - protein, 8 aa, Homo sapiens
MQGIHLLT

SEQ ID NO: 348 - protein, 8 aa, Homo sapiens
QHYYGTPH

SEQ ID NO: 349 - protein, 8 aa, Homo sapiens
QQYNTYPT

SEQ ID NO: 350 - protein, 8 aa, Homo sapiens
QEFGDSGT

SEQ ID NO: 351 - protein, 8 aa, Homo sapiens
QQYGGSPW

SEQ ID NO: 352 - protein, 8 aa, Homo sapiens
QQYGSSRT

SEQ ID NO: 353 - protein, 8 aa, Homo sapiens
QQYDSLPT

SEQ ID NO: 354 - protein, 8 aa, Homo sapiens
QQYGSVFT

SEQ ID NO: 355 - protein, 8 aa, Homo sapiens
QQYNSYCS

SEQ ID NO: 356 - protein, 8 aa, Homo sapiens
QQYYSTPL

SEQ ID NO: 357 - protein, 8 aa, Homo sapiens
QQYNDWPT

SEQ ID NO: 358 - protein, 8 aa, Homo sapiens
MQNIQFPT

SEQ ID NO: 359 - protein, 8 aa, Homo sapiens
QQYDNLPP

SEQ ID NO: 360 - protein, 8 aa, Homo sapiens
  Unknown (5)..(5)
  misc_feature (5)..(5): Xaa can be any naturally occurring amino acid
QLLRXLRT

SEQ ID NO: 361 - protein, 8 aa, Homo sapiens
YQYNNGYT

SEQ ID NO: 362 - protein, 8 aa, Homo sapiens
QQRSNWPT

SEQ ID NO: 363 - protein, 8 aa, Homo sapiens
QQYGTSHT

SEQ ID NO: 364 - protein, 8 aa, Homo sapiens
QQYNHWPS

SEQ ID NO: 365 - protein, 8 aa, Homo sapiens
QQYGSLYT

SEQ ID NO: 366 - protein, 8 aa, Homo sapiens
QQNKDWPL

SEQ ID NO: 367 - protein, 8 aa, Homo sapiens
QQFGTSLT

SEQ ID NO: 368 - protein, 8 aa, Homo sapiens
QQRSNWWT

SEQ ID NO: 369 - protein, 8 aa, Homo sapiens
QQCSNWPT

SEQ ID NO: 370 - protein, 8 aa, Homo sapiens
QQYGSSPT

SEQ ID NO: 371 - protein, 10 aa, Homo sapiens
QQYGSSPSIT

SEQ ID NO: 372 - protein, 10 aa, Homo sapiens
QKYNSAPPST

SEQ ID NO: 373 - protein, 10 aa, Homo sapiens
QEYNNWPLWT

SEQ ID NO: 374 - protein, 10 aa, Homo sapiens
QQYGGSPPWT

SEQ ID NO: 375 - protein, 10 aa, Homo sapiens
HEYNGWPPWT

SEQ ID NO: 376 - protein, 10 aa, Homo sapiens
QQYNSYSPLT

SEQ ID NO: 377 - protein, 10 aa, Homo sapiens
MQHTHWSPIT

SEQ ID NO: 378 - protein, 10 aa, Homo sapiens
QHYNNWPPWT

SEQ ID NO: 379 - protein, 10 aa, Homo sapiens
QQSYNTPPWT

SEQ ID NO: 380 - protein, 10 aa, Homo sapiens
QQSYNTPPWT

SEQ ID NO: 381 - protein, 10 aa, Homo sapiens
QHYGSSPPWT

SEQ ID NO: 382 - protein, 10 aa, Homo sapiens
QQHNNWPPLT

SEQ ID NO: 383 - protein, 10 aa, Homo sapiens
QVYGQSPVFT

SEQ ID NO: 384 - protein, 10 aa, Homo sapiens
QQYGSSPMYT

SEQ ID NO: 385 - protein, 10 aa, Homo sapiens
QQYGSSPMYT

SEQ ID NO: 386 - protein, 10 aa, Homo sapiens
QRFGTSPLYT

SEQ ID NO: 387 - protein, 10 aa, Homo sapiens
QQYGDSPLYS

SEQ ID NO: 388 - protein, 10 aa, Homo sapiens
QQYDDWPPIT

SEQ ID NO: 389 - protein, 10 aa, Homo sapiens
QQLNSYPPYT

SEQ ID NO: 390 - protein, 10 aa, Homo sapiens
QQSYSTPPDT

SEQ ID NO: 391 - protein, 10 aa, Homo sapiens
QHYNNWPPSS

SEQ ID NO: 392 - protein, 10 aa, Homo sapiens
QHYNRLPPWT

SEQ ID NO: 393 - protein, 10 aa, Homo sapiens
QQYDRSVPLT

SEQ ID NO: 394 - protein, 10 aa, Homo sapiens
QQYYTTPTYT

SEQ ID NO: 395 - protein, 10 aa, Homo sapiens
QQYYTTPPLT

SEQ ID NO: 396 - protein, 10 aa, Homo sapiens
QQLYSYPHLT

SEQ ID NO: 397 - protein, 10 aa, Homo sapiens
CQQYGSSRWT

SEQ ID NO: 398 - protein, 10 aa, Homo sapiens
MQALQTPMST

SEQ ID NO: 399 - protein, 10 aa, Homo sapiens
QQRSEWPPLT

SEQ ID NO: 400 - protein, 10 aa, Homo sapiens
QQYDTSPAWT

SEQ ID NO: 401 - protein, 10 aa, Homo sapiens
QQYGSSQGFT

SEQ ID NO: 402 - protein, 10 aa, Homo sapiens
MQSIQLPRWT

SEQ ID NO: 403 - protein, 10 aa, Homo sapiens
QHYGLSPPIT

SEQ ID NO: 404 - protein, 10 aa, Homo sapiens
QEYGSSPPRT

SEQ ID NO: 405 - protein, 10 aa, Homo sapiens
SSYRSSSTRV

SEQ ID NO: 406 - protein, 10 aa, Homo sapiens
QHYGLSPPIT

SEQ ID NO: 407 - protein, 10 aa, Homo sapiens
QEYGSSPPRT

SEQ ID NO: 408 - protein, 10 aa, Homo sapiens
QQYYTTLPLT

SEQ ID NO: 409 - protein, 10 aa, Homo sapiens
SSYSSTTRVV

SEQ ID NO: 410 - protein, 10 aa, Homo sapiens
QQYGSSPQTF

SEQ ID NO: 411 - protein, 10 aa, Homo sapiens
FCQYNRYPYT

SEQ ID NO: 412 - protein, 10 aa, Homo sapiens
LQRSNWGEVT

SEQ ID NO: 413 - protein, 10 aa, Homo sapiens
QQRSNWGEVT

SEQ ID NO: 414 - protein, 10 aa, Homo sapiens
QQRSNWGEVT

SEQ ID NO: 415 - protein, 10 aa, Homo sapiens
QQYGSSPLFT

SEQ ID NO: 416 - protein, 10 aa, Homo sapiens
CSYTSSSTLV

SEQ ID NO: 417 - protein, 10 aa, Homo sapiens
QQRSNWPPIT

SEQ ID NO: 418 - protein, 10 aa, Homo sapiens
QQSYNTLSLT

SEQ ID NO: 419 - protein, 10 aa, Homo sapiens
QHYGNSPPYT

SEQ ID NO: 420 - protein, 10 aa, Homo sapiens
QQSHKTLAWT

SEQ ID NO: 421 - protein, 10 aa, Homo sapiens
MQGTYWPPYT

SEQ ID NO: 422 - protein, 10 aa, Homo sapiens
HQYYTYPLFT

SEQ ID NO: 423 - protein, 10 aa, Homo sapiens
HQYYTYPLFT

SEQ ID NO: 424 - protein, 10 aa, Homo sapiens
QQSYSTPPWT

SEQ ID NO: 425 - protein, 10 aa, Homo sapiens
QQSYTNPEVT

SEQ ID NO: 426 - protein, 10 aa, Homo sapiens
QQYGSSPPYT

SEQ ID NO: 427 - protein, 10 aa, Homo sapiens
QQYGSSPRYT

SEQ ID NO: 428 - protein, 10 aa, Homo sapiens
QQYGSSPRYT

SEQ ID NO: 429 - protein, 10 aa, Homo sapiens
  Unknown (10)..(10)
  misc_feature (10)..(10): Xaa can be any naturally occurring amino acid
QQFGNSPPLX

SEQ ID NO: 430 - protein, 10 aa, Homo sapiens
QQYAGSPPVT

SEQ ID NO: 431 - protein, 10 aa, Homo sapiens [sequence omitted]

SEQ ID NO: 432 - protein, 10 aa, Homo sapiens [sequence omitted]

SEQ ID NO: 433 - protein, 10 aa, Homo sapiens
QQRSNCSGLT

SEQ ID NO: 434 - protein, 10 aa, Homo sapiens [sequence omitted]

SEQ ID NO: 435 - protein, 10 aa, Homo sapiens [sequence omitted]

SEQ ID NO: 436 - protein, 10 aa, Homo sapiens
QQYNNWPPCT

SEQ ID NO: 437 - protein, 10 aa, Homo sapiens [sequence omitted]

SEQ ID NO: 438 - protein, 10 aa, Homo sapiens
QQRSFWPPLT

SEQ ID NO: 439 - protein, 10 aa, Homo sapiens
QQRSNWPSIT

SEQ ID NO: 440 - protein, 10 aa, Homo sapiens
QQRSNWPPLT

SEQ ID NO: 441 - protein, 10 aa, Homo sapiens
QQRTNWPIFT

SEQ ID NO: 442 - protein, 10 aa, Homo sapiens
QQRSNWPPGT

SEQ ID NO: 443 - protein, 10 aa, Homo sapiens
QQYNNWPPLT

SEQ ID NO: 444 - protein, 10 aa, Homo sapiens
QQYNNWPTWT

SEQ ID NO: 445 - protein, 10 aa, Homo sapiens
QQRMRWPPLT

SEQ ID NO: 446 - protein, 10 aa, Homo sapiens
QQYGSSPKWT

SEQ ID NO: 447 - protein, 10 aa, Homo sapiens
QQYGSSPQYT

SEQ ID NO: 448 - protein, 10 aa, Homo sapiens
QQYGSSPPYT

SEQ ID NO: 449 - protein, 10 aa, Homo sapiens
QQYDRSLPRT

SEQ ID NO: 450 - protein, 10 aa, Homo sapiens
QQYGNSPLFS

SEQ ID NO: 451 - protein, 10 aa, Homo sapiens
QQYGGSPLFS

SEQ ID NO: 452 - protein, 10 aa, Homo sapiens
QQYNNWPTWT

SEQ ID NO: 453 - protein, 10 aa, Homo sapiens
MQALQTLGLT

SEQ ID NO: 454 - protein, 10 aa, Homo sapiens
MQALQTLGLT

SEQ ID NO: 455 - protein, 10 aa, Homo sapiens
QQSHSAPPYT

SEQ ID NO: 456 - protein, 10 aa, Homo sapiens
QQYGSSPLFT

SEQ ID NO: 457 - protein, 10 aa, Homo sapiens
QQYNDWPPWT

SEQ ID NO: 458 - protein, 10 aa, Homo sapiens
QQYNGNSPLT

SEQ ID NO: 459 - protein, 10 aa, Homo sapiens
QQLNTYPPWT

SEQ ID NO: 460 - protein, 10 aa, Homo sapiens
HKYGGSPPYT

SEQ ID NO: 461 - protein, 10 aa, Homo sapiens
MQDTHWPPWT

SEQ ID NO: 462 - protein, 10 aa, Homo sapiens
QHYGRSPPLT

SEQ ID NO: 463 - protein, 10 aa, Homo sapiens
QQYGNSPPYT

SEQ ID NO: 464 - protein, 10 aa, Homo sapiens
QQYGSSPPYT

SEQ ID NO: 465 - protein, 10 aa, Homo sapiens
QQYFNVPPVT

SEQ ID NO: 466 - protein, 10 aa, Homo sapiens
QHYHNLPPTT

SEQ ID NO: 467 - protein, 10 aa, Homo sapiens
IQGTHWPQYT

SEQ ID NO: 468 - protein, 10 aa, Homo sapiens
QQYGSSRALT

SEQ ID NO: 469 - protein, 10 aa, Homo sapiens
QQYYSTPSYT

SEQ ID NO: 470 - protein, 10 aa, Homo sapiens
MQALQTLMCS

SEQ ID NO: 471 - protein, 10 aa, Homo sapiens
QQSYSTPPLT

SEQ ID NO: 472 - protein, 10 aa, Homo sapiens
QQSYSTPPIT

SEQ ID NO: 473 - protein, 10 aa, Homo sapiens
QQYGGSLPIT

SEQ ID NO: 474 - protein, 10 aa, Homo sapiens
QQYGSSTPLT

SEQ ID NO: 475 - protein, 10 aa, Homo sapiens
QQRSSWPPLT

SEQ ID NO: 476 - protein, 10 aa, Homo sapiens
QQRYSWPPLT

SEQ ID NO: 477 - protein, 10 aa, Homo sapiens
QQRYNWPPLT

SEQ ID NO: 478 - protein, 10 aa, Homo sapiens
QQRSNWPPLT

SEQ ID NO: 479 - protein, 10 aa, Homo sapiens
QQRSSWPPLT

SEQ ID NO: 480 - protein, 10 aa, Homo sapiens [sequence omitted]

SEQ ID NO: 481 - protein, 10 aa, Homo sapiens
QQRSNWPPYT

SEQ ID NO: 482 - protein, 10 aa, Homo sapiens [sequence omitted]

SEQ ID NO: 483 - protein, 10 aa, Homo sapiens [sequence omitted]

SEQ ID NO: 484 - protein, 10 aa, Homo sapiens
QQYGSSPPIT

SEQ ID NO: 485 - protein, 10 aa, Homo sapiens
QQYNNWPPIT

SEQ ID NO: 486 - protein, 10 aa, Homo sapiens
QQRSSWPPIT

SEQ ID NO: 487 - protein, 10 aa, Homo sapiens
QQYGSSPRVT

SEQ ID NO: 488 - protein, 10 aa, Homo sapiens
QQYNTNSPIS

SEQ ID NO: 489 - protein, 10 aa, Homo sapiens
QNYGSSPRIT

SEQ ID NO: 490 - protein, 10 aa, Homo sapiens
QQYGSSPPIT

SEQ ID NO: 491 - protein, 10 aa, Homo sapiens
MQSIQLPRFT

SEQ ID NO: 492 - protein, 10 aa, Homo sapiens
MQSVQLPRFT

SEQ ID NO: 493 - protein, 10 aa, Homo sapiens
MQSVQLPRFT

SEQ ID NO: 494 - protein, 10 aa, Homo sapiens
QQYDKWPPVT

SEQ ID NO: 495 - protein, 10 aa, Homo sapiens
MQSIQFPRWT

SEQ ID NO: 496 - protein, 10 aa, Homo sapiens
MQGIHLPPYI

SEQ ID NO: 497 - protein, 10 aa, Homo sapiens
NQGTQWLLYT

SEQ ID NO: 498 - protein, 10 aa, Homo sapiens
QQYNSYAPYT

SEQ ID NO: 499 - protein, 10 aa, Homo sapiens
QHYGLSPPIT

SEQ ID NO: 500 - protein, 10 aa, Homo sapiens
QEYGSSPPRT

SEQ ID NO: 501 - protein, 10 aa, Homo sapiens
QQYFNVPPVT

SEQ ID NO: 502 - protein, 10 aa, Homo sapiens
QQLTSYPPWT

SEQ ID NO: 503 - protein, 10 aa, Homo sapiens
QQVNSYPGLT

SEQ ID NO: 504 - protein, 10 aa, Homo sapiens
QQVFSYPGIT

SEQ ID NO: 505 - protein, 10 aa, Homo sapiens
QQYTSLPGIT

SEQ ID NO: 506 - protein, 10 aa, Homo sapiens
QHSYSTLPLT

SEQ ID NO: 507 - protein, 10 aa, Homo sapiens
QQYYNIPYIT

SEQ ID NO: 508 - protein, 10 aa, Homo sapiens
QLYGSSPRVT

SEQ ID NO: 509 - protein, 10 aa, Homo sapiens
QQYANWPPIT

SEQ ID NO: 510 - protein, 10 aa, Homo sapiens
QQYNISPRDT

SEQ ID NO: 511 - protein, 10 aa, Homo sapiens
QQFGSSPLIT

SEQ ID NO: 512 - protein, 10 aa, Homo sapiens
QQYGDFPRVT

SEQ ID NO: 513 - protein, 10 aa, Homo sapiens
QQYGDWPPYT

SEQ ID NO: 514 - protein, 10 aa, Homo sapiens
QQYYTTLSWT

SEQ ID NO: 515 - protein, 10 aa, Homo sapiens
QQYNKWPPLT

SEQ ID NO: 516 - protein, 10 aa, Homo sapiens
MQGTHWLPVT

SEQ ID NO: 517 - protein, 10 aa, Homo sapiens
QQYDKWPPVT

SEQ ID NO: 518 - protein, 10 aa, Homo sapiens
QQYDNLPPIH

SEQ ID NO: 519 - protein, 10 aa, Homo sapiens
QQLNNYPPFT

SEQ ID NO: 520 - protein, 10 aa, Homo sapiens
QQSYSTPPYT

SEQ ID NO: 521 - protein, 10 aa, Homo sapiens
QQSYSTPPYS

SEQ ID NO: 522 - protein, 10 aa, Homo sapiens
QQSYSTPPYT

SEQ ID NO: 523 - protein, 10 aa, Homo sapiens
QQYNNWLPFT

SEQ ID NO: 524 - protein, 10 aa, Homo sapiens
AAWDDSLTLM

SEQ ID NO: 525 - protein, 22 aa, Homo sapiens
EVQLVESGGGLVQPGGSLKLSC

SEQ ID NO: 526 - protein, 11 aa, Homo sapiens
LSCVASGFIFS

SEQ ID NO: 527 - protein, 6 aa, Homo sapiens
FSNHWM

SEQ ID NO: 528 - protein, 18 aa, Homo sapiens
HWMNWVRQAPGKGLEWVA

SEQ ID NO: 529 - protein, 5 aa, Homo sapiens
IRSKS

SEQ ID NO: 530 - protein, 5 aa, Homo sapiens
ATHYA

SEQ ID NO: 531 - protein, 15 aa, Homo sapiens
HYAESVKGRFTISRD

SEQ ID NO: 532 - protein, 12 aa, Homo sapiens
RFTISRDDSKSI

SEQ ID NO: 533 - protein, 6 aa, Homo sapiens
IVYLQM

SEQ ID NO: 534 - protein, 8 aa, Homo sapiens
YLQMTDLR

SEQ ID NO: 535 - protein, 11 aa, Homo sapiens
LRTEDTGVYYC

SEQ ID NO: 536 - protein, 8 aa, Homo sapiens
VYYCSRNY

SEQ ID NO: 537 - protein, 5 aa, Homo sapiens
NYYGS

SEQ ID NO: 538 - protein, 4 aa, Homo sapiens
GSTY

SEQ ID NO: 539 - protein, 9 aa, Homo sapiens
TYDYWGQGT

SEQ ID NO: 540 - protein, 13 aa, Homo sapiens
DYWGQGTTVTVSS

SEQ ID NO: 541 - protein, 6 aa, Homo sapiens
DILLTQ

SEQ ID NO: 542 - protein, 20 aa, Homo sapiens
LTQSPAILSLSPGERATLSC

SEQ ID NO: 543 - protein, 17 aa, Homo sapiens
LSLSPGERATLSCRASQ

SEQ ID NO: 544 - protein, 4 aa, Homo sapiens
VGSS

SEQ ID NO: 545 - protein, 7 aa, Homo sapiens
IHWYQQK

SEQ ID NO: 546 - protein, 13 aa, Homo sapiens
QQKPNQSPKLLIK

SEQ ID NO: 547 - protein, 7 aa, Homo sapiens
LLIKYAS

SEQ ID NO: 548 - protein, 4 aa, Homo sapiens
YASE

SEQ ID NO: 549 - protein, 21 aa, Homo sapiens
PSRFSGSGSGTDFTLTINSLE

SEQ ID NO: 550 - protein, 8 aa, Homo sapiens
SLESEDAA

SEQ ID NO: 551 - protein, 7 aa, Homo sapiens
ADYYCQQ

SEQ ID NO: 552 - protein, 8 aa, Homo sapiens
YYCQQSHS

SEQ ID NO: 553 - protein, 4 aa, Homo sapiens
HSWP

SEQ ID NO: 554 - protein, 9 aa, Homo sapiens
WPFTFGQGT

SEQ ID NO: 555 - protein, 11 aa, Homo sapiens
TFGQGTNLEIK

SEQ ID NO: 556 - DNA, 24 bp, Artificial
  Primer
atgtacaacactgtgtcatttaac

SEQ ID NO: 557 - DNA, 30 bp, Artificial
  Primer
ttatttggagcttttaaacttaaggatacc

SEQ ID NO: 558 - protein, 15 aa, Bougainvillea spectabilis
AKVDRKDLELGVYKL

SEQ ID NO: 559 - protein, 5 aa, Homo sapiens
AAKAD

SEQ ID NO: 560 - protein, 5 aa, Homo sapiens
AKADR

SEQ ID NO: 561 - protein, 5 aa, Homo sapiens
KADRK

SEQ ID NO: 562 - protein, 6 aa, Homo sapiens
AAKSDR

SEQ ID NO: 563 - protein, 6 aa, Homo sapiens
KSDRKD

SEQ ID NO: 564 - protein, 5 aa, Homo sapiens
AAKTD

SEQ ID NO: 565 - protein, 5 aa, Homo sapiens
AKTDR

SEQ ID NO: 566 - protein, 5 aa, Homo sapiens
KTDRK

SEQ ID NO: 567 - protein, 15 aa, Bougainvillea spectabilis
LGVYKLEFSIEAIHG

SEQ ID NO: 568 - protein, 5 aa, Homo sapiens
ELGPQ

SEQ ID NO: 569 - protein, 5 aa, Homo sapiens
LGPQK

SEQ ID NO: 570 - protein, 6 aa, Homo sapiens
GPQKLE

SEQ ID NO: 571 - protein, 5 aa, Homo sapiens
ELGGK

SEQ ID NO: 572 - protein, 6 aa, Homo sapiens
LGGKKL

SEQ ID NO: 573 - protein, 6 aa, Homo sapiens
GGKKLE

SEQ ID NO: 574 - protein, 5 aa, Homo sapiens
ELGNS

SEQ ID NO: 575 - protein, 6 aa, Homo sapiens
LGNSKL

SEQ ID NO: 576 - protein, 6 aa, Homo sapiens
GNSKLE

SEQ ID NO: 577 - protein, 7 aa, Homo sapiens
ELGQAKL

SEQ ID NO: 578 - protein, 7 aa, Homo sapiens
LGQAKLE

SEQ ID NO: 579 - protein, 5 aa, Homo sapiens
ELGQD

SEQ ID NO: 580 - protein, 5 aa, Homo sapiens
LGQDK

SEQ ID NO: 581 - protein, 5 aa, Homo sapiens
QDKLE

SEQ ID NO: 582 - protein, 15 aa, Bougainvillea spectabilis
NGQEIAKFFLIVIQM

SEQ ID NO: 583 - protein, 5 aa, Homo sapiens
GQEQA

SEQ ID NO: 584 - protein, 5 aa, Homo sapiens
QEQAK

SEQ ID NO: 585 - protein, 5 aa, Homo sapiens
EQAKF

SEQ ID NO: 586 - protein, 5 aa, Homo sapiens
GQERA

SEQ ID NO: 587 - protein, 5 aa, Homo sapiens
QERAK

SEQ ID NO: 588 - protein, 5 aa, Homo sapiens
ERAKF

SEQ ID NO: 589 - protein, 15 aa, Hirudo medicinalis
CILGSDGEKNQCVTG

SEQ ID NO: 590 - protein, 4 aa, Homo sapiens
KCRH

SEQ ID NO: 591 - protein, 15 aa, Artificial
  Modified Peptide
CRHGSDGEKNQCVTG

SEQ ID NO: 592 - protein, 20 aa, Homo sapiens
GGSNNLSCLTIPASANNGGS

SEQ ID NO: 593 - protein, 120 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 594 - protein, 108 aa, Homo sapiens
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKR

SEQ ID NO: 595 - protein, 121 aa, Homo sapiens
EVQLVESGGGVVQPGRSLRLSCSSSGFTFSDYYMYWVRQAPGKGLEWVAYMSNVGAITDYPDTVKGRFTISRDNAKSTLYLQMDSLRPEDTGVYFCARGTRDGSWFAYAVWGQGTPVTVSS

SEQ ID NO: 596 - protein, 112 aa, Homo sapiens
DIQMTQSPSSLSASVGDRVTITCRSSQRIVHSNGNTYLEWYQQTPGKAPKLLIYKVSNRFSGVPSRFSGSGSGTDFTFTISSLQPEDIATYYCFQGSHVPFTFGQGTKLQIT

SEQ ID NO: 597 - protein, 120 aa, Homo sapiens
EVQLVESGGGLVQPGGSLRLSCAVSGYSITSGYSWNWIRQAPGKGLEWVAITYDGSTNYNPSVKGRITISRDDSKNTFYLQMNSLRAEDTAVYYCARGSHYFGHWHFAVWGQGTLVTVSS

SEQ ID NO: 598 - protein, 111 aa, Homo sapiens [sequence omitted]

SEQ ID NO: 599 - protein, 192 aa, Homo sapiens
EVKLVESGGGLVQPGGSMRLSCVASGFTFSNHWMNWVRQAPGKGLEWVAEIRSKSINSATHYAESVKGRFTISRDDSKSSVYLQMNDLKTEDTGVEVKLEESGGGLVQPGGSMKLSCVASGFIFSNHWMNWYRQSPEKGLEWVAEIRSKSINSATHYAESVKGGRFTISRDDSKSAVYLQMTDLRTEDTGVY

SEQ ID NO: 600 - protein, 200 aa, Homo sapiens
DILLTQSPDIQSVTPKEKVTITCRASQFGSSIHWYQQKTDQSPKLLIKYASESMSGIPSRFSGSGSGTDFTLTINSLESEDAADYYCQQSHSWPFTFGQDILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSG